(19) United States
(12) Patent Application Publication
(10) Pub. No.: US 2015/0291514 A1
(43) Pub. Date: Oct. 15, 2015

(54) N-Aminosulfonyl Benzamides

(71) Applicant: Pfizer Limited, Sandwich, Kent (GB)

(72) Inventors: Alan Daniel Brown, Great Abington (GB); Sebastien Rene Gabriel Galan, Sandwich (UK); David Simon Millan, Sandwich (UK); David James Rawson, Sandwich (UK); Robert Ian Storer, Great Abington (UK); Paul Anthony Stupple, Sandwich (UK); NIgel Alan Swain, Great Abington (US)

(73) Assignee: PFIZER LIMTED, Sandwich, Kent (GB)

(21) Appl. No.: 14/358,603

(22) PCT Filed: Dec. 21, 2012
(86) PCT No.: PCT/IB2012/057625

§ 371 Date: May 15, 2014

Related U.S. Application Data
(60) Provisional application No. 61/582,916, filed on Jan. 4, 2012.

(51) Int. Cl. C07C 307/06; C07D 213/69; A61K 31/44; C07D 237/08; A61K 31/50; A61K 31/397; A61K 31/4439; C07D 213/74; A61K 31/4427; C07D 213/65; C07D 205/04; A61K 31/165; C07D 401/12

(52) U.S. Cl. CPC C07C 307/06; A61K 31/165; C07D 213/69; A61K 31/44; C07D 237/08; A61K 31/50; C07D 401/12; A61K 31/4439; C07D 213/74; A61K 31/4427; C07D 213/65; C07D 205/04; A61K 31/397; C07B 59/002

(57) ABSTRACT

The present invention relates to sulfonamide derivatives of formula (I):

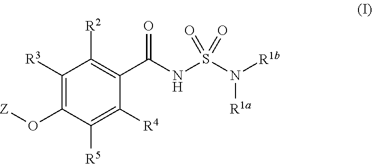

or a pharmaceutically acceptable salts thereof, wherein Z, R^{1a}, R^{1b}, R², R³, R⁴ and R⁵ are as defined in the description, and to their use in medicine, to compositions containing them, to processes for their preparation and to intermediates used in such processes. The compounds of formula (I) are Nav1.7 inhibitors useful in the treatment of a wide range of disorders, particularly pain.

The invention relates to sulfonamide derivatives, to their use in medicine, to compositions containing them, to processes for their preparation and to intermediates used in such processes.

Voltage-gated sodium channels are found in all excitable cells including myocytes of muscle and neurons of the central and peripheral nervous system. In neuronal cells, sodium channels are primarily responsible for generating the rapid upstroke of the action potential. In this manner sodium channels are essential to the initiation and propagation of electrical signals in the nervous system. Proper and appropriate function of sodium channels is therefore necessary for normal function of the neuron. Consequently, aberrant sodium channel function is thought to underlie a variety of medical disorders (see Hubner C A, Jentsch T J, Hum. Mol. Genet., 11(20): 2435-45 (2002) for a general review of inherited ion channel disorders) including epilepsy (Yogeeswari et al., Curr. Drug Targets, 5(7): 589-602 (2004)), arrhythmia (Noble D., Proc. Natl. Acad. Sci. USA, 99(9): 5755-6 (2002)) myotonia (Cannon, S C, Kidney Int 57(3): 772-9 (2000)), and pain (Wood, J N et al., J. Neurobiol., 61(1): 55-71 (2004)).

There are currently at least nine known members of the family of voltage-gated sodium channel (VGSC) alpha subunits. Names for this family include SCNx, SCNAx, and Naᵥx.x. The VGSC family has been phylogenetically divided into two subfamilies Naᵥ1.x (all but SCN6A) and Naᵥ2.x (SCN6A). The Nav1.x subfamily can be functionally subdivided into two groups, those which are sensitive to blocking by tetrodotoxin (TTX-sensitive or TTX-s) and those which are resistant to blocking by tetrodotoxin (TTX-resistant or TTX-r).

The Naᵥ1.7 (PN1, SCN9A) VGSC is sensitive to blocking by tetrodotoxin and is preferentially expressed in peripheral sympathetic and sensory neurons. The SCN9A gene has been cloned from a number of species, including human, rat, and rabbit and shows ˜90% amino acid identity between the human and rat genes (Toledo-Aral et al., Proc. Natl. Acad. Sci. USA, 94(4): 1527-1532 (1997)).

An increasing body of evidence suggests that Naᵥ1.7 may play a key role in various pain states, including acute, inflammatory and/or neuropathic pain. Deletion of the SCN9A gene in nociceptive neurons of mice led to a reduction in mechanical and thermal pain thresholds and reduction or abolition of inflammatory pain responses (Nassar et al., Proc Natl Acad Sci USA, 101(34): 12706-11 (2004)). In humans, Naᵥ1.7 protein has been shown to accumulate in neuromas, particularly painful neuromas (Kretschmer et al., Acta. Neurochir. (Wien), 144(8): 803-10 (2002)). Gain of function mutations of Naᵥ1.7, both familial and sporadic, have been linked to primary erythermalgia, a disease characterized by burning pain and inflammation of the extremities (Yang et al., J. Med. Genet., 41(3): 171-4 (2004), and paroxysmal extreme pain disorder (Waxman, S G Neurology. 7;69(6): 505-7 (2007)). Congruent with this observation is the report that the non-selective sodium channel blockers lidocaine and mexiletine can provide symptomatic relief in cases of familial erythermalgia (Legroux-Crepel et al., Ann. Dermatol Venereol., 130: 429-433) and carbamazepine is effective in reducing the number and severity of attacks in PEPD (Fertleman et al, Neuron.; 52(5):767-74 (2006). Further evidence of the role of Nav1.7 in pain is found in the phenotype of loss of function mutations of the SCN9A gene. Cox and colleagues (Nature, 444(7121):894-8 (2006)) were the first to report an association between loss-of-function mutations of SNC9A and congenital indifference to pain (CIP), a rare autosomal recessive disorder characterized by a complete indifference or insensitivity to painful stimuli. Subsequent studies have revealed a number of different mutations that result in a loss of function of the SCN9A gene and and the CIP phenotype (Goldberg et al, Clin Genet.; 71(4): 311-9 (2007), Ahmad et al, Hum Mol Genet. 1;16(17): 2114-21 (2007)).

Nav1.7 inhibitors are therefore potentially useful in the treatment of a wide range of disorders, particularly pain, including: acute pain; chronic pain; neuropathic pain; inflammatory pain; visceral pain; nociceptive pain including post-surgical pain; and mixed pain types involving the viscera, gastrointestinal tract, cranial structures, musculoskeletal system, spine, urogenital system, cardiovascular system and CNS, including cancer pain, back and orofacial pain.

Certain inhibitors of voltage gated sodium channels useful in the treatment of pain are known. Thus WO-A-2005/013914 discloses heteroarylamino sulfonylphenyl derivatives, WO-A-2008/118758 aryl sulphonamides, WO-A-2009/012242 N-thiazolyl benzenesulfonamides and WO-A-2010/079443 aryl sulphonamides.

There is, however, an ongoing need to provide new Naᵥ1.7 inhibitors that are good drug candidates.

Prefererably compounds are selective Nav1.7 channel inhibitors. That is, preferred compounds show an affinity for the Nav1.7 channel over other Nav channels. In particular, they should show an affinity for the Nav1.7 channel which is greater than their affinity for Nav1.5 channels. Advantageously, compounds should show little or no affinity for the Nav1.5 channel.

Selectivity for the Nav1.7 channel over Nav1.5 may potentially lead to one or more improvements in side-effect profile. Without wishing to be bound by theory, such selectivity is thought to reduce any cardiovascular side effects which may be associated with affinity for the Nav1.5 channel. Preferably compounds demonstrate a selectivity of 10-fold, more preferably 30-fold, most preferably 100-fold, for the Nav1.7 channel when compared to their selectivity for the Nav1.5 channel whilst maintaining good potency for the Nav1.7 channel.

Furthermore, preferred compounds should have one or more of the following properties: be well absorbed from the gastrointestinal tract; be metabolically stable; have a good metabolic profile, in particular with respect to the toxicity or allergenicity of any metabolites formed; or possess favourable pharmacokinetic properties whilst still retaining their activity profile as Nav1.7 channel inhibitors. They should be non-toxic and demonstrate few side-effects. Ideal drug candidates should exist in a physical form that is stable, non-hygroscopic and easily formulated.

We have now found new sulphonamide Nav1.7 inhibitors.

According to a first aspect of the invention there is provided a compound of formula (I)

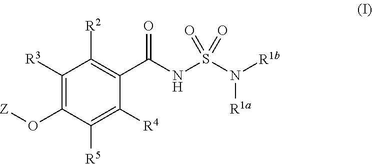

or a pharmaceutically acceptable salt thereof, wherein

Z is a group selected from naphthyl, phenyl and Het¹, said group being optionally independently substituted by one to three substituents selected from Y¹ and Y²;

Y¹ and Y² are independently selected from F; Cl; CN; (C₁-C₈)alkyl, optionally substituted by (C₃-C₈)cycloalkyl and/or, valency permitting, by one to eight F; (C₃-C₈)cycloalkyl, optionally substituted, valency permitting, by one to eight F; NR⁷R⁸; (C₁-C₈)alkyloxy, optionally independently substituted by one to three R⁹, and/or, valency permitting, by one to eight F; (C₃-C₈)cycloalkyloxy, optionally independently substituted, valency permitting, by one to eight F and/or by one to three R¹⁰, and further optionally fused to a phenyl ring; phenyl, optionally independently substituted by one to three substituents selected from F and R¹⁰; phenoxy, optionally independently substituted by one to three substituents selected from F and R¹⁰; Het²; Het²-oxy; and Het³;

R^{1a} and R^{1b} are independently H; (C₁-C₆)alkyl; or (C₃-C₆)cycloalkyl, optionally substituted, valency permitting, by one to eight F; or, taken together with the N atom to which they are attached, form a 3- to 8-membered monoheterocycloalkyl, said monoheterocycloalkyl being optionally substituted on a ring carbon atom by, valency permitting, one to eight F;

R², R³, R⁴ are independently H, F, Cl or —OCH₃;

R⁵ is H, CN, F, Cl, Het³, or R⁶;

R⁶ is a group selected from (C₁-C₆)alkyl and (C₁-C₆)alkyloxy, wherein each group is optionally substituted, valency permitting, by one to eight F;

R⁷ and R⁸ are independently selected from H; (C₁-C₈)alkyl, optionally independently substituted by one to three R¹¹; (C₃-C₈)cycloalkyl, optionally substituted by, valency permitting, one to eight F and/or by one to three R¹⁰, and further optionally fused to a phenyl ring; (C₅-C₈)bridged bicycloalkyl; ‘C-linked’ Het²; and C-linked Het³; or, taken together with the N atom to which they are attached, form a 3- to 8-membered monoheterocycloalkyl, said monoheterocycloalkyl being optionally substituted on a ring carbon atom by (C₁-C₆)alkyl and/or, valency permitting, one to two F;

R⁹ is (C₁-C₆)alkyloxy; (C₃-C₈)cycloalkyl, optionally substituted, valency permitting, by one to eight F; Het²; or phenyl, optionally independently substituted by one to three R⁶;

R¹⁰ is Cl, CN or R⁶;

R¹¹ is F; (C₁-C₈)alkyloxy; (C₃-C₈)cycloalkyl, optionally substituted, valency permitting, by one to eight F; ‘C-linked’ Het²; or phenyl, optionally independently substituted by one to three R⁶;

Het¹ is a 6-, 9- or 10-membered heteroaryl containing one to three nitrogen atoms;

Het² is a 3- to 8-membered saturated monoheterocycloalkyl containing one or two ring members selected from —NR¹²—and —O—, said monoheterocycloalkyl being optionally substituted on a ring carbon atom by one to three substituents independently selected from F, (C₁-C₈)alkyl, (C₁-C₄)alkyloxy(C₀-C₄)alkylene and (C₃-C₈)cycloalkyl;

Het³ is a 5- or 6-membered heteroaryl containing one to three nitrogen atoms, said heteroaryl being optionally substituted by one to three substituents selected from F, Cl, CN and R⁶; and

R¹² is H, (C₁-C₈)alkyl or (C₃-C₈)cycloalkyl, wherein (C₁-C₈)alkyl and (C₃-C₈)cycloalkyl are optionally substituted, valency permitting, by one to eight F; or, when Het² is ‘N-linked’, is absent.

Described below are a number of embodiments (E) of this first aspect of the invention, where for convenience E1 is identical thereto.
E1 A compound of formula (I) as defined above or a pharmaceutically acceptable salt thereof.
E2 A compound according to E1 wherein Z is phenyl optionally independently substituted by one to three substituents selected from Y¹ and Y².
E3 A compound according to E1 or E2 wherein Z is phenyl optionally independently substituted by one or two substituents selected from Y¹ and Y².
E4 A compound according to E3 wherein said phenyl is meta and para substituted.
E5 A compound according to E1 wherein Z is a 6-membered heteroaryl comprising one to three nitrogen atoms, said heteroaryl being optionally independently substituted by one to three substituents selected from Y¹ and Y².
E6 A compound according to E1 or E5 wherein Z is pyridyl optionally independently substituted by one to three substituents selected from Y¹ and Y².
E7 A compound according to any of E1, E5 or E6 wherein Z is pyridyl optionally independently substituted by one or two substituents selected from Y¹ and Y².
E8 A compound according to any of E1 or E5 to E7 wherein Z is pyridyl optionally independently substituted by one or two substituents selected from Y¹ and Y² and wherein said pyridyl is orientated as below:

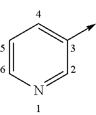

E9 A compound according to E8 wherein said pyridyl is 6-substituted or, where di-substituted, 5- and 6-substituted.
E10 A compound according to any of E1 to E9 wherein Y¹ and Y² are independently selected from F; Cl; (C₁-C₆)alkyl, optionally substituted by, valency permitting, one to six F; NR⁷R⁸ wherein R⁷ and R⁸ are independently selected from H, (C₁-C₆)alkyl, (C₃-C₆)cycloalkyl, (C₅-C₈)bridged bicycloalkyl or (C₁-C₆)alkyl substituted by (C₃-C₆)cycloalkyl; NR⁷R⁸ wherein R⁷ and R⁸ taken together with the N atom to which they are attached form a 3- to 8-membered monoheterocycloalkyl, said monoheterocycloalkyl being optionally substituted on a ring carbon atom by (C₁-C₆)alkyl and/or, valency permitting, one to two F; (C₁-C₈)alkyloxy, optionally substituted by (C_{3—}C₆)cycloalkyl, and/or, valency permitting, one to eight F; phenyl; and Het³.
E11 A compound according to any of E1 to E10 wherein Y¹ and Y² are independently selected from F; Cl; (C₁-C₃)alkyl, optionally substituted by one to three F; NR⁷R⁸ wherein R⁷ and R⁸ are independently selected from H, (C₁-C₃)alkyl, (C₃-C₅)cycloalkyl, (C₅-C₆)bridged bicycloalkyl or (C₁-C₃)alkyl substituted by (C₃-C₅)cycloalkyl; NR⁷R⁸ wherein R⁷ and R⁸ taken together with the N atom to which they are attached form a 3- to 6-membered monoheterocycloalkyl, said monoheterocycloalkyl being optionally substituted on a ring carbon atom by (C₁-C₃)alkyl and/or, valency permitting, one to two F; (C₁-C₆)alkyloxy, optionally substituted by, valency permitting, one to six F.
E12 A compound according to any of E1 to 11 wherein Y¹ and Y² are independently selected from Cl; (C₁-C₂)alkyl, optionally substituted by one to three F; NR⁷R⁸ wherein R⁷ and R⁸ are independently selected from H, (C₁-C₃)alkyl, (C₃-C₄)cycloalkyl or (C₁-C₃)alkyl substituted by (C₃-C₄)cycloalkyl; and (C₁-C₄)alkyloxy, optionally substituted by, valency permitting, one to six F.
E13 A compound according to any of E1 to E12 wherein R^{1a} and R^{1b} are independently selected from H or (C₁-C₃)alkyl; or, taken together with the N atom to which they are attached, form a 3- to 6-membered monoheterocycloalkyl, said monoheterocycloalkyl being optionally substituted on a ring carbon atom by one or two F.
E14 A compound according to any of E1 to E13 wherein R^{1a} and R^{1b} are independently selected from H or methyl; or, taken together with the N atom to which they are attached, form a 3- to 6-membered monoheterocycloalkyl.
E15 A compound according to any of E1 to E14 wherein R^{1a} and R^{1b} are methyl.
E16 A compound according to any of E1 to E15 wherein R², R³, R⁴ and R⁵ are independently H, F or Cl.
E17 A compound according to any of E1 to E16 wherein R², R³, R⁴ and R⁵ are H.
E18 A compound according to any of E1 to E16 wherein R² and R⁵ are independently selected from F or Cl, and R³ and R⁴ are both H.
E19 A compound according to any of E1 to E15, or E18, wherein R² is F, R³ and R⁴ are both H; and R⁵ is F or Cl.
E20 A compound according to E1 selected from:
   4-[4-chloro-3-(trifluoromethyl)phenoxy]-N-[(dimethylamino)sulfonyl]benzamide;
   4-[(5-chloro-6-isobutoxypyridin-3-yl)oxy]-N-[(dimethylamino)sulfonyl]-2,5-difluorobenzamide;
   4-[4-chloro-3-(trifluoromethyl)phenoxy]-N-[(methylamino)sulfonyl]benzamide;
   N-(aminosulfonyl)-4-(4-chloro-2-methoxyphenoxy)benzamide;
   4-(4-chloro-2-methoxyphenoxy)-N-[(dimethylamino)sulfonyl]benzamide;
   N-(aminosulfonyl)-4-(4-chloro-2-methoxyphenoxy)-2,5-difluorobenzamide;
   4-(4-chloro-2-methoxyphenoxy)-N-[(dimethylamino)sulfonyl]-2,5-difluorobenzamide;
   N-[(dimethylamino)sulfonyl]-4-(2-methoxyphenoxy)benzamide diethylamine salt;
   N-[(dimethylamino)sulfonyl]-4-(3-methoxyphenoxy)benzamide diethylamine salt;
   N-(aminosulfonyl)-4-[(5-chloro-6-isobutoxypyridin-3-yl)oxy]benzamide diethylamine salt;
   4-[(5-chloro-6-isobutoxypyridin-3-yl)oxy]-N-[(dimethylamino)sulfonyl]benzamide diethylamine salt;
   5-chloro-N-[(dimethylamino)sulfonyl]-2-fluoro-4-(2-methoxyphenoxy)benzamide;
   N-[(dimethylamino)sulfonyl]-2,5-difluoro-4-(2-methoxyphenoxy)benzamide;
   N-[(dimethylamino)sulfonyl]-2,5-difluoro-4-(3-methoxyphenoxy)benzamide diethylamine salt;
   N-(aminosulfonyl)-4-(4-chloro-2-pyridazin-4-ylphenoxy)benzamide;
   4-(4-chloro-2-pyridazin-4-ylphenoxy)-N-[(dimethylamino)sulfonyl]benzamide;
   4-[(5-chloro-6-isopropoxypyridin-3-yl)oxy]-N-[(dimethylamino)sulfonyl]-2,5-difluorobenzamide-d7;
   N-(aminosulfonyl)-4-(biphenyl-2-yloxy)-3-cyanobenzamide;
   4-(biphenyl-2-yloxy)-3-cyano-N-[(ethylamino)sulfonyl]benzamide;
   4-{[5-chloro-6-(3-fluoropyrrolidin-1-yl)pyridine-3-yl]oxy}-N-[(dimethylamino)sulfonyl]-2,5-difluorobenzamide;
   4-({5-chloro-6-[(cyclopropylmethyl)amino]pyridine-3-yl}oxy)-N-[(dimethylamino)sulfonyl]-2,5-difluorobenzamide;
   4-{[5-chloro-6-(dimethylamino)pyridine-3-yl]oxy}-N-[(dimethylamino)sulfonyl]-2,5-difluorobenzamide;
   4-({5-chloro-6-[(cyclopropylmethyl)(methyl)amino]pyridine-3-yl}oxy)-N-[(dimethylamino)sulfonyl]-2,5-difluorobenzamide;
   4-({5-chloro-6-[isopropyl(methyl)amino]pyridine-3-yl}oxy)-N-[(dimethylamino)sulfonyl]-2,5-difluorobenzamide;
   4-{[5-chloro-6-(methylamino)pyridine-3-yl]oxy}-N-[(dimethylamino)sulfonyl]-2,5-difluorobenzamide;
   4-[(5-chloro-6-pyrrolidin-1-ylpyridin-3-yl)oxy]-N-[(dimethylamino)sulfonyl]-2,5-difluorobenzamide;
   4-{[5-chloro-6-(cyclopropylamino)pyridin-3-yl]oxy}-N-[(dimethylamino)sulfonyl]-2,5-difluorobenzamide;
   4-{[6-(pyridin[1.1.1]pent-1-ylamino)-5-chloropyridin-3-yl]oxy}-N-[(dimethylamino)sulfonyl]-2,5-difluorobenzamide;
   4-({5-chloro-6-[(2R)-2-methylpyrrolidin-1-yl]pyridine-3-yl}oxy)-N-[(dimethylamino)sulfonyl]-2,5-dfluorobenzamide;
   4-[(5-chloro-6-isopropoxypyridin-3-yl)oxy]-N-[(dimethylamino)sulfonyl]-2,5-difluorobenzamide;
   4-[(5-chloro-6-isopropoxypyridin-3-yl)oxy]-N-[(methylamino)sulfonyl]-2,5-difluorobenzamide;
   4-[(5-chloro-6-(cyclopropylmethoxy)pyridin-3-yl)oxy]-N-[(dimethylamino)sulfonyl]-2,5-difluorobenzamide;
   4-[(5-chloro-6-(2-fluoro-2-methylpropoxy)pyridin-3-yl)oxy]-N-[(dimethylamino)sulfonyl]-2,5-difluorobenzamide;
   4-[(5-chloro-6-(2,2,2-trifluoroethoxy)pyridin-3-yl)oxy]-N-[(dimethylamino)sulfonyl]-2,5-difluorobenzamide;
   4-((5-chloro-4-(trifluoromethyl)pyridin-2-yl)oxy)-N-(N,N-dimethylsulfamoyl)-2,5-difluorobenzamide;
   4-((5-chloro-6-((1,1,1-trifluoropropan-2-yl)oxy)pyridin-3-yl)oxy)-N-[(dimethylamino)sulfonyl]-2,5-difluorobenzamide;
   4-((5-chloro-6-((1,1,1,3,3,3-hexafluoropropan-2-yl)oxy)pyridin-3-yl)oxy)-N-[(dimethylamino)sulfonyl]-2,5-difluorobenzamide;
   N-(azetidin-1-ylsulfonyl)-4-((5-chloro-6-isopropoxypyridin-3-yl)oxy)-2,5-difluorobenzamide;
   N-(aminosulfonyl)-4[4-chloro-3-(trifluoromethyl)phenoxy]benzamide;
   or a pharmaceutically acceptable salt thereof.
E21 A compound according to E1 selected from:
   4-{[5-chloro-6-(2,2,3,3,3-pentafluoropropoxy)pyridin-3-yl]oxy}-N-[(dimethylamino)sulfonyl]-2,5-difluorobenzamide;
   5-chloro-4-[3-chloro-4-(trifluoromethyl)phenoxy]-N-[(dimethylamino)sulfonyl]-2-fluorobenzamide;
   5-chloro-4-[3-chloro-4-(trifluoromethyl)phenoxy]-N-[(3,3-difluoroazetidin-1-yl)sulfonyl]-2-fluorobenzamide;
   N-(azetidin-1-ylsulfonyl)-5-chloro-4-[3-chloro-4-(trifluoromethyl)phenoxy]-2-fluorobenzamide;
   5-chloro-4-[4-chloro-3-(trifluoromethyl)phenoxy]-N-[(dimethylamino)sulfonyl]-2-fluorobenzamide;
   5-chloro-4-[4-chloro-3-(trifluoromethyl)phenoxy]-N-[(3,3-difluoroazetidin-1-yl)sulfonyl]-2-fluorobenzamide;
   5-chloro-4-[3-chloro-4-(trifluoromethoxy)phenoxy]-N-[(dimethylamino)sulfonyl]-2-fluorobenzamide;
   5-chloro-4-[3-chloro-4-(trifluoromethoxy)phenoxy]-N-[(3,3-difluoroazetidin-1-yl)sulfonyl]-2-fluorobenzamide;
   5-chloro-4-[4-chloro-3-(trifluoromethoxy)phenoxy]-N-[(dimethylamino)sulfonyl]-2-fluorobenzamide;
   N-(azetidin-1-ylsulfonyl)-5-chloro-4-[4-chloro-3-(trifluoromethyl)phenoxy]-2-fluorobenzamide;
   5-chloro-4-[4-chloro-3-(trifluoromethoxy)phenoxy]-N-[(3,3-difluoroazetidin-1-yl)sulfonyl]-2-fluorobenzamide;
   N-(azetidin-1-ylsulfonyl)-5-chloro-4-[3-chloro-4-(trifluoromethoxy)phenoxy]-2-fluorobenzamide;
   5-chloro-4-(3,4-dichlorophenoxy)-N-[(dimethylamino)sulfonyl]-2-fluorobenzamide;
   5-chloro-4-(3,4-dichlorophenoxy)-N-[(3,3-difluoroazetidin-1-yl)sulfonyl]-2-fluorobenzamide;
   N-(azetidin-1-ylsulfonyl)-5-chloro-4-[4-chloro-3-(trifluoromethoxy)phenoxy]-2-fluorobenzamide;
   5-chloro-4-{[5-chloro-6-(2,2,2-trifluoro-1,1-dimethylethoxy)pyridin-3-yl]oxy}-N-[(dimethylamino)sulfonyl]-2-fluorobenzamide;
   N-(azetidin-1-ylsulfonyl)-5-chloro-4-(3,4-dichlorophenoxy)-2-fluorobenzamide;
   5-chloro-4-{[5-chloro-6-(2,2,3,3-tetrafluoropropoxy)pyridin-3-yl]oxy}-N-[(dimethylamino)sulfonyl]-2-fluorobenzamide;
   N-(azetidin-1-ylsulfonyl)-5-chloro-4-{[5-chloro-6-(2,2,2-trifluoro-1,1-dimethylethoxy)pyridin-3-yl]oxy}-2-fluorobenzamide;
   5-chloro-4-{[5-chloro-6-(2,2,3,3,3-pentafluoropropoxy)pyridin-3-yl]oxy}-N-[(dimethylamino)sulfonyl]-2-fluorobenzamide;
   5-chloro-4-{[5-chloro-6-(2,2,2-trifluoroethoxy)pyridin-3-yl]oxy}-N-[(dimethylamino)sulfonyl]-2-fluorobenzamide;
   N-(azetidin-1-ylsulfonyl)-5-chloro-4-{[5-chloro-6-(2,2,3,3-tetrafluoropropoxy)pyridin-3-yl]oxy}-2-fluorobenzamide;
   5-chloro-4-{[5-chloro-6-(2,2,3,3,3-pentafluoropropoxy)pyridin-3-yl]oxy}-N-[(3,3-difluoroazetidin-1-yl)sulfonyl]-2-fluorobenzamide;
   N-(azetidin-1-ylsulfonyl)-5-chloro-4-{[5-chloro-6-(2,2,2-trifluoroethoxy)pyridin-3-yl]oxy}-2-fluorobenzamide;
   5-chloro-4-{[5-chloro-6-(2,2,2-trifluoro-1,1-dimethylethoxy)pyridin-3-yl]oxy}-N-[(3,3-difluoroazetidin-1-yl)sulfonyl]-2-fluorobenzamide;
   N-(azetidin-1-ylsulfonyl)-5-chloro-4-{[5-chloro-6-(2,2,3,3,3-pentafluoropropoxy)pyridin-3-yl]oxy}-2-fluorobenzamide;
   5-chloro-4-{[5-chloro-6-(2,2,3,3-tetrafluoropropoxy)pyridin-3-yl]oxy}-N-[(3,3-difluoroazetidin-1-yl)sulfonyl]-2-fluorobenzamide;
   5-chloro-4-{[5-chloro-6-(2,2,2-trifluoroethoxy)pyridin-3-yl]oxy}-N-[(3,3-difluoroazetidin-1-yl)sulfonyl]-2-fluorobenzamide;
   N-(azetidin-1-ylsulfonyl)-4-{[5-chloro-6-(2,2,3,3,3-pentafluoropropoxy)pyridin-3-yl]oxy}-2,5-difluorobenzamide;
   4-{[5-chloro-6-(2,2,3,3,3-pentafluoropropoxy)pyridin-3-yl]oxy}-N-[(3,3-difluoroazetidin-1-yl)sulfonyl]-2,5-difluorobenzamide;
   5-chloro-4-{[5-chloro-6-(2,2,2-trifluoro-1-methylethoxy)pyridin-3-yl]oxy}-N-[(dimethylamino)sulfonyl]-2-fluorobenzamide;
   5-chloro-4-{[5-chloro-6-(2,2,2-trifluoro-1-methylethoxy)pyridin-3-yl]oxy}-N-[(3,3-difluoroazetidin-1-yl)sulfonyl]-2-fluorobenzamide;
   N-(azetidin-1-ylsulfonyl)-5-chloro-4-{[5-chloro-6-(2,2,2-trifluoro-1-methylethoxy)pyridin-3-yl]oxy}-2-fluorobenzamide;
   or a pharmaceutically acceptable salt thereof.

Alkyl, alkylene, and alkoxy groups, containing the requisite number of carbon atoms, can be unbranched or branched. Examples of alkyl include methyl, ethyl, n-propyl, i-propyl, n-butyl, i-butyl, sec-butyl and t-butyl. Examples of alkoxy include methoxy, ethoxy, n-propoxy, i-propoxy, n-butoxy, i-butoxy, sec-butoxy and t-butoxy. Examples of alkylene include methylene, 1, 1-ethylene, 1, 2-ethylene, 1, 1-propylene, 1, 2-propylene, 1, 3-propylene and 2, 2-propylene.

Examples of cycloalkyl include cyclopropyl, cyclobutyl, cyclopentyl, cyclohexyl, cycloheptyl and cyclooctyl.

Examples of bridged bicycloalkyl include bicyclo[1.1.1]pentane, bicyclo[2.1.1]hexane, bicyclo[2.2.1]heptane, bicyclo[2.2.2]octane and bicyclo[3.2.1]octane.

Halo means fluoro, chloro, bromo or iodo.

The term ‘C-linked’ used in the definitions of formula (I) means that the group in question is joined via a ring carbon. The term ‘N-linked’ used in the definitions of formula (I) means that the group in question is joined via a ring nitrogen.

Specific examples of 5- or 6-membered heteroaryl used in the definitions of formula (I) include pyrrolyl, pyrazolyl, imidazoyl, pyridyl, pyridazinyl, pyrimidinyl and pyrazinyl.

Except as expressly defined above, when such heteroaryls are substituted, the substituent may be located on a ring carbon (in all cases) or a ring nitrogen with appropriate valency (if the substituent is joined through a carbon atom).

Specific examples of 9- or 10-membered heteroaryl used in the definitions of formula (I) include indolyl, benzimidazolyl, indazolyl, benzotriazolyl, pyrrolo[2,3-b]pyridyl, pyrrolo[2,3-c]pyridyl, pyrrolo[3,2-c]pyridyl, pyrrolo[3,2-b]pyridyl, imidazo[4,5-b]pyridyl, imidazo[4,5-c]pyridyl, pyrazolo[4,3-d]pyridyl, pyrazolo[4,3-c]pyridyl, pyrazolo[3,4-c]pyridyl, pyrazolo[3,4-b]pyridyl, isoindolyl, indazolyl, purinyl, indolizinyl, imidazo[1,2-a]pyridyl, imidazo[1,5-a]pyridyl, pyrazolo[1,5-a]pyridyl, pyrrolo[1,2-b]pyridazinyl, imidazo[1,2-c]pyrimidinyl, quinolinyl, isoquinolinyl, cinnolinyl, quinazolinyl, quinoxalinyl, phthalazinyl, 1,6-naphthyridinyl, 1,7-naphthyridinyl, 1,8-naphthyridinyl, 1,5-naphthyridinyl, 2,6-naphthyridinyl, 2,7-naphthyridinyl, pyrido[3,2-d]pyrimidinyl, pyrido[4,3-d]pyrimidinyl, pyrido[3,4-d]pyrimidinyl, pyrido[2,3-d]pyrimidinyl, pyrido[2,3-d]pyrazinyl and pyrido[3,4-b]pyrazinyl. Except as expressly defined above, when such heteroaryls are substituted, the substituent may be located on a ring carbon (in all cases) or a ring nitrogen with appropriate valency (if the substituent is joined through a carbon atom).

Specific examples of Het² include oxiranyl, aziridinyl, oxetanyl, azetidinyl, tetrahydrofuranyl, pyrrolidinyl, tetrahydropyranyl, piperidinyl, morpholinyl, piperazinyl, azepanyl, oxepanyl, oxazepanyl and diazepinyl.

Hereinafter, all references to compounds of the invention include compounds of formula (I) or pharmaceutically acceptable salts, solvates, or multi-component complexes thereof, or pharmaceutically acceptable solvates or multi-component complexes of pharmaceutically acceptable salts of compounds of formula (I), as discussed in more detail below.

Preferred compounds of the invention are compounds of formula (I) or pharmaceutically acceptable salts thereof.

Suitable acid addition salts are formed from acids which form non-toxic salts. Examples include the acetate, adipate, aspartate, benzoate, besylate, bicarbonate/carbonate, bisulphate/sulphate, borate, camsylate, citrate, cyclamate, edisylate, esylate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mesylate, methylsulphate, naphthylate, 2-napsylate, nicotinate, nitrate, orotate, oxalate, palmitate, pamoate, phosphate/hydrogen phosphate/dihydrogen phosphate, pyroglutamate, saccharate, stearate, succinate, tannate, tartrate, tosylate, trifluoroacetate and xinofoate salts.

Suitable base salts are formed from bases which form non-toxic salts. Examples include the aluminium, arginine, benzathine, calcium, choline, diethylamine, diolamine, glycine, lysine, magnesium, meglumine, olamine, potassium, sodium, tromethamine and zinc salts.

Hemisalts of acids and bases may also be formed, for example, hemisulphate and hemicalcium salts.

The skilled person will appreciate that the aforementioned salts include ones wherein the counterion is optically active, for example d-lactate or l-lysine, or racemic, for example dl-tartrate or dl-arginine.

For a review on suitable salts, see “Handbook of Pharmaceutical Salts: Properties, Selection, and Use” by Stahl and Wermuth (Wiley-VCH, Weinheim, Germany, 2002).

Pharmaceutically acceptable salts of compounds of formula (I) may be prepared by one or more of three methods:
(i) by reacting the compound of formula (I) with the desired acid or base;
(ii) by removing an acid- or base-labile protecting group from a suitable precursor of the compound of formula (I) using the desired acid or base; or
(iii) by converting one salt of the compound of formula (I) to another by reaction with an appropriate acid or base or by means of a suitable ion exchange column.

All three reactions are typically carried out in solution. The resulting salt may precipitate out and be collected by filtration or may be recovered by evaporation of the solvent. The degree of ionisation in the resulting salt may vary from completely ionised to almost non-ionised.

The compounds of formula (I) or pharmaceutically acceptable salts thereof may exist in both unsolvated and solvated forms. The term ‘solvate’ is used herein to describe a molecular complex comprising a compound of formula (I) or a pharmaceutically acceptable salt thereof and one or more pharmaceutically acceptable solvent molecules, for example, ethanol. The term ‘hydrate’ is employed when said solvent is water. Pharmaceutically acceptable solvates in accordance with the invention include those wherein the solvent of crystallization may be isotopically substituted, e.g. D₂O, d₆-acetone and d₆-DMSO.

A currently accepted classification system for organic hydrates is one that defines isolated site, channel, or metal-ion coordinated hydrates—see Polymorphism in Pharmaceutical Solids by K. R. Morris (Ed. H. G. Brittain, Marcel Dekker, 1995), incorporated herein by reference. Isolated site hydrates are ones in which the water molecules are isolated from direct contact with each other by intervening organic molecules. In channel hydrates, the water molecules lie in lattice channels where they are next to other water molecules. In metal-ion coordinated hydrates, the water molecules are bonded to the metal ion.

When the solvent or water is tightly bound, the complex will have a well-defined stoichiometry independent of humidity. When, however, the solvent or water is weakly bound, as in channel solvates and hygroscopic compounds, the water/solvent content will be dependent on humidity and drying conditions. In such cases, non-stoichiometry will be the norm.

The compounds of the invention may exist in a continuum of solid states ranging from fully amorphous to fully crystalline. The term ‘amorphous’ refers to a state in which the material lacks long range order at the molecular level and, depending upon temperature, may exhibit the physical properties of a solid or a liquid. Typically such materials do not give distinctive X-ray diffraction patterns and, while exhibiting the properties of a solid, are more formally described as a liquid. Upon heating, a change from solid to liquid properties occurs which is characterised by a change of state, typically second order (‘glass transition’). The term ‘crystalline’ refers to a solid phase in which the material has a regular ordered internal structure at the molecular level and gives a distinctive X-ray diffraction pattern with defined peaks. Such materials when heated sufficiently will also exhibit the properties of a liquid, but the change from solid to liquid is characterised by a phase change, typically first order (‘Melting point’).

Also included within the scope of the invention are multi-component complexes (other than salts and solvates) of compounds of formula (I) or pharmaceutically acceptable salts thereof wherein the drug and at least one other component are present in stoichiometric or non-stoichiometric amounts. Complexes of this type include clathrates (drug-host inclusion complexes) and co-crystals. The latter are typically defined as crystalline complexes of neutral molecular constituents which are bound together through non-covalent interactions, but could also be a complex of a neutral molecule with a salt. Co-crystals may be prepared by melt crystallisation, by recrystallisation from solvents, or by physically grinding the components together—see Chem Commun, 17, 1889-1896, by O. Almarsson and M. J. Zaworotko (2004), incorporated herein by reference. For a general review of multi-component complexes, see J Pharm Sci, 64 (8), 1269-1288, by Haleblian (August 1975), incorporated herein by reference.

The compounds of the invention may also exist in a mesomorphic state (mesophase or liquid crystal) when subjected to suitable conditions. The mesomorphic state is intermediate between the true crystalline state and the true liquid state (either melt or solution). Mesomorphism arising as the result of a change in temperature is described as ‘thermotropic’ and that resulting from the addition of a second component, such as water or another solvent, is described as ‘lyotropic’. Compounds that have the potential to form lyotropic mesophases are described as ‘amphiphilic’ and consist of molecules which possess an ionic (such as —COO⁻Na⁺, —COO⁶³¹ K⁺, or —SO₃⁻Na⁺) or non-ionic (such as —N⁻N⁺(CH₃)₃) polar head group. For more information, see Crystals and the Polarizing Microscope by N. H. Hartshorne and A. Stuart, 4^{th} Edition (Edward Arnold, 1970), incorporated herein by reference.

The compounds of the invention may be administered as prodrugs. Thus certain derivatives of compounds of formula (I) which may have little or no pharmacological activity themselves can, when administered into or onto the body, be converted into compounds of formula (I) having the desired activity, for example, by hydrolytic cleavage. Such derivatives are referred to as ‘prodrugs’. Further information on the use of prodrugs may be found in ‘Pro-drugs as Novel Delivery Systems, Vol. 14, ACS Symposium Series (T Higuchi and W Stella) and ‘Bioreversible Carriers in Drug Design’, Pergamon Press, 1987 (ed. E B Roche, American Pharmaceutical Association).

Prodrugs can, for example, be produced by replacing appropriate functionalities present in a compound of formula (I) with certain moieties known to those skilled in the art as ‘pro-moieties’ as described, for example, in “Design of Prodrugs” by H Bundgaard (Elsevier, 1985).

Examples of prodrugs include phosphate prodrugs, such as dihydrogen or dialkyl (e.g. di-tert-butyl) phosphate prodrugs. Further examples of replacement groups in accordance with the foregoing examples and examples of other prodrug types may be found in the aforementioned references.

Also included within the scope of the invention are metabolites of compounds of formula (I), that is, compounds formed in vivo upon administration of the drug. Some examples of metabolites in accordance with the invention include, where the compound of formula (I) contains a phenyl (Ph) moiety, a phenol derivative thereof (-Ph>-PhOH);

Compounds of the invention containing one or more asymmetric carbon atoms can exist as two or more stereoisomers. Included within the scope of the invention are all stereoisomers of the compounds of the invention and mixtures of one or more thereof.

Conventional techniques for the preparation/isolation of individual enantiomers include chiral synthesis from a suitable optically pure precursor or resolution of the racemate (or the racemate of a salt or derivative) using, for example, chiral high pressure liquid chromatography (HPLC).

Alternatively, the racemate (or a racemic precursor) may be reacted with a suitable optically active compound, for example, an alcohol, or, in the case where the compound of formula (I) contains an acidic or basic moiety, a base or acid such as 1-phenylethylamine or tartaric acid. The resulting diastereomeric mixture may be separated by chromatography and/or fractional crystallization and one or both of the diastereoisomers converted to the corresponding pure enantiomer(s) by means well known to a skilled person.

Chiral compounds of the invention (and chiral precursors thereof) may be obtained in enantiomerically-enriched form using chromatography, typically HPLC, on an asymmetric resin with a mobile phase consisting of a hydrocarbon, typically heptane or hexane, containing from 0 to 50% by volume of isopropanol, typically from 2% to 20%, and from 0 to 5% by volume of an alkylamine, typically 0.1% diethylamine. Concentration of the eluate affords the enriched mixture.

Mixtures of stereoisomers may be separated by conventional techniques known to those skilled in the art; see, for example, “Stereochemistry of Organic Compounds” by E. L. Eliel and S. H. Wilen (Wiley, New York, 1994.

The scope of the invention includes all crystal forms of the compounds of the invention, including racemates and racemic mixtures (conglomerates) thereof. Stereoisomeric conglomerates may also be separated by the conventional techniques described herein just above.

The scope of the invention includes all pharmaceutically acceptable isotopically-labelled compounds of the invention wherein one or more atoms are replaced by atoms having the same atomic number, but an atomic mass or mass number different from the atomic mass or mass number which predominates in nature.

Examples of isotopes suitable for inclusion in the compounds of the invention include isotopes of hydrogen, such as ²H and ³H, carbon, such as ¹¹C, ¹³C and ¹⁴C, chlorine, such as ³⁶Cl, fluorine, such as ¹⁸F, iodine, such as ¹²³I and ¹²⁵I, nitrogen, such as ¹³N and ¹⁵N, oxygen, such as ¹⁵O, ¹⁷O and ¹⁸O, phosphorus, such as ³²P, and sulphur, such as ³⁵S.

Certain isotopically-labelled compounds of the invention, for example, those incorporating a radioactive isotope, are useful in drug and/or substrate tissue distribution studies. The radioactive isotopes tritium, i.e. ³H, and carbon-14, i.e. ¹⁴C, are particularly useful for this purpose in view of their ease of incorporation and ready means of detection. Substitution with heavier isotopes such as deuterium, i.e. ²H, may afford certain therapeutic advantages resulting from greater metabolic stability, for example, increased in vivo half-life or reduced dosage requirements, and hence may be preferred in some circumstances. Substitution with positron emitting isotopes, such as ₁₁C, ¹⁸F, ¹⁵O and ¹³N, can be useful in Positron Emission Topography (PET) studies for examining substrate receptor occupancy.

Isotopically-labeled compounds of formula (I) can generally be prepared by conventional techniques known to those skilled in the art or by processes analogous to those described in the accompanying Examples and Preparations using an appropriate isotopically-labeled reagent in place of the non-labeled reagent previously employed.

Also within the scope of the invention are intermediate compounds as hereinafter defined, all salts, solvates and complexes thereof and all solvates and complexes of salts thereof as defined hereinbefore for compounds of formula (I). The invention includes all polymorphs of the aforementioned species and crystal habits thereof.

When preparing a compound of formula (I) in accordance with the invention, a person skilled in the art may routinely select the form of intermediate which provides the best combination of features for this purpose. Such features include the melting point, solubility, processability and yield of the intermediate form and the resulting ease with which the product may be purified on isolation.

The compounds of the invention may be prepared by any method known in the art for the preparation of compounds of analogous structure. In particular, the compounds of the invention can be prepared by the procedures described by reference to the Schemes that follow, or by the specific methods described in the Examples, or by similar processes to either.

The skilled person will appreciate that the experimental conditions set forth in the schemes that follow are illustrative of suitable conditions for effecting the transformations shown, and that it may be necessary or desirable to vary the precise conditions employed for the preparation of compounds of formula (I). It will be further appreciated that it may be necessary or desirable to carry out the transformations in a different order from that described in the schemes, or to modify one or more of the transformations, to provide the desired compound of the invention.

In addition, the skilled person will appreciate that it may be necessary or desirable at any stage in the synthesis of compounds of the invention to protect one or more sensitive groups, so as to prevent undesirable side reactions. In particular, it may be necessary or desirable to protect amino or carboxylic acid groups. The protecting groups used in the preparation of the compounds of the invention may be used in conventional manner. See, for example, those described in ‘Greene's Protective Groups in Organic Synthesis’ by Theodora W Greene and Peter G M Wuts, third edition, (John Wiley and Sons, 1999), in particular chapters 7 (“Protection for the Amino Group”) and 5 (“Protection for the Carboxyl Group”), incorporated herein by reference, which also describes methods for the removal of such groups.

In the following general methods, R^{1a}, R^{1b}, R², R³, R⁴, R⁵, R⁷, R⁸, R⁹, R¹⁰ and Z are as previously defined for a derivative of the formula (I) unless otherwise stated. Pg is a suitable carboxylic acid protecting group such as tert butyl, methyl, ethyl, or tolyl. E is nitrile. Lg is a suitable leaving group, such as halo (e.g. Br) or a sulphonate ester (e.g mesylate, triflate or tosylate).

Where ratios of solvents are given, the ratios are by volume.

According to a first process, compounds of formula (I), may be prepared by the process illustrated in Scheme 1.

Compounds of formula (I) can be made from compounds of formula (III) according to process step (v) by displacement of the ester with compounds of formula (VI), optionally in the presence of a suitable base. Suitable conditions include potassium tert-butoxide in THF at 60° C., NaH in THF at 65° C. and potassium carbonate and 1,8-diazabicyclo[5.4.0]undec-7-ene (DBU) In DMSO at 50° C. Preferred conditions comprise heating in DMSO at 60° C. for 18 hours.

Alternatively compounds of formula (I) can be made from compounds of formula (II) according to reaction step (vi) by activation of the acid group with reagents such as oxalyl chloride, carbonyl di-imidazole (CDI), a uronium based peptide coupling agent or a carbodiimide reagent followed by displacement with a sulfamide of formula (VI) in the presence of a nucleophilic base, such as 4-dimethylaminopyridine. Preferred conditions comprise N,N-dimethylaminopropyl-N′-ethylcarbodiimide and 4-dimethylaminopyridine in DCM or N-[(dimethylamino)(3H-[1,2,3]triazolo[4,5-b]pyridin-3-yloxy)methylene]-N-methylmethanaminium hexafluorophosphate and N-ethyl-N-isopropylpropan-2-amine in DCM or 2,4,6-tripropyl-1,3,5,2,4,6-trioxatriphosphinane 2,4,6-trioxide and N-ethyl-N-isopropylpropan-2-amine in THF at 65° C.

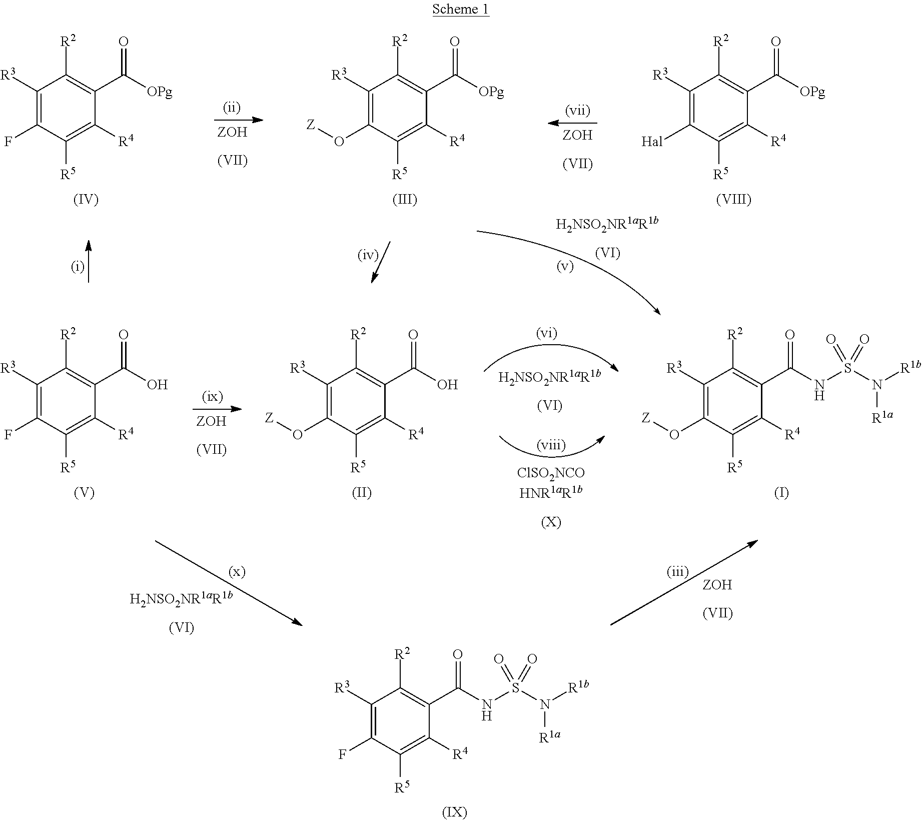

Compounds of formula (I) may also be prepared from compounds of formula (II) according to process step (viii), by reaction with chlorosulfonylisocyanate and amines of formula (X). Preferred conditions comprise heating compounds of formula (II) with chlorosulfonylisocyanate in DCM at 50° C. followed by stirring in acetonitrile with amines of formula (X) at room temperature.

Compounds of formula (I) can also be made from compounds of formula (IX) according to process step (iii) by nucleophilic aromatic substitution reaction (SnAr) using an alcohol of formula (VII) in the presence of a base. Typical conditions for process step (iii) include potassium carbonate in DMF or DMSO; sodium hydride in NMP or DMF; sodium hydroxide or potassium hydroxide in 1,4-dioxane and water or DMSO; potassium tert-butoxide in THF; or cesium carbonate and copper powder in pyridine at 120° C. Preferred conditions comprise potassium carbonate in DMSO at from 80° C. to 170° C.

Compounds of formula (III) can be made from compounds of formula (IV) according to process step (ii) by a nucleophilic aromatic substitution reaction (SnAr) using compounds of formula (VII) and base. Suitable conditions are described above in process step (iii). Preferred conditions comprise 2 equivalents of potassium carbonate in DMSO at room temperature.

Alternatively, compounds of formula (III) can also be prepared from halides of formula (VIII) according to process step (vii) by reaction with compounds of formula (VII) under copper catalysed conditions. Typical conditions comprise copper iodide and potassium phosphate in DMSO at 90° C.

Compounds of formula (IV) can be prepared from compounds of formula (V) according to process step (i) using protecting group methodology as referred to above in ‘Greene's Protective Groups in Organic Synthesis’. When Pg is tolyl, preferred conditions comprise thionyl chloride at 50° C. using para-cresol. When Pg is tert-butyl, preferred conditions comprise di-tert-butyldicarbonate and 4-dimethylaminopyridine in tert-butanol.

Compounds of formula (II) can be made from compounds of formula (III) according to process step (iv) by hydrolysis of the ester under basic or acidic conditions. Preferred conditions are sodium hydroxide in a mixture of MeOH and THF or lithium hydroxide in a mixture of THF and water at from room temperature to 55° C. or TFA in DCM at room temperature.

Alternatively compounds of formula (II) can be made from compounds of formula (V) according to process step (ix) by a nucleophilic aromatic substitution reaction (SNAr) using compounds of formula (VII) and base as described for process step (iii) at elevated temperatures. Preferred conditions comprise potassium carbonate in DMSO at 90° C.

Compounds of formula (IX) can be prepared from compounds of formula (V) according to process step (x) by employing a sulfamide of formula (VI) under conditions described above in process step (vi).

According to a second process, compounds of formula (I) may be prepared by the process illustrated in Scheme 2.

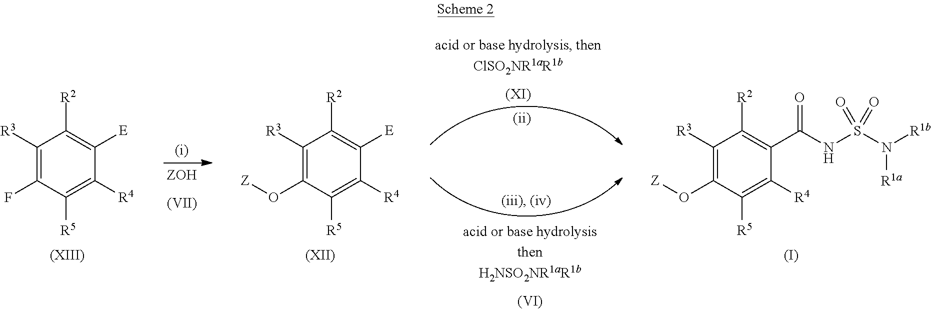

Compounds of formula (I) can be prepared from compounds of formula (XII) according to reaction step (ii) by acid or base hydrolysis of the nitrile to the primary carboxamide, followed by reaction with an appropriate sulfamoyl chloride of formula (XI). Preferred conditions comprise hydrogen peroxide and potassium carbonate in DMSO, followed by lithium or sodium hexamethyldisilazide in THF, at a temperature from room temperature to 60° C.

Alternatively, compounds of formula (I) can be prepared from compounds of formula (XII) according to reaction step (iii), by hydrolysis of the nitrile by either acidic or basic methods to the carboxylic acid, followed by displacement with a sulfamide of formula (VI) according to process step (iv). Typical conditions for process step (iii) are as described for step (iv) in Scheme 1; preferred conditions comprise potassium hydroxide in water and ethylene glycol at 120° C. Preferred conditions for process step (iv) are as described for the corresponding step (vi) in Scheme 1.

Compounds of formula (XII) can be prepared from compounds of formula (XIII) according to process step (i) by a nucleophilic aromatic substitution reaction (SNAr) with compounds of formula (VII) and base, using conditions described in Scheme 1 for the corresponding process step (ii) or (iii).

According to a third process, compounds of formula (I), wherein Y¹ is selected from NR⁷R⁸; (C₁-C₈)alkyloxy, optionally independently substituted by one to three R⁹, and/or, valency permitting, by one to eight F; and (C₃-C₈)cycloalkyloxy, optionally independently substituted, valency permitting, by one to eight F and/or by one to three R¹⁰; may be prepared by interconversion from the corresponding compounds of formula (I) wherein Y¹ is F by the process illustrated in Scheme 3.

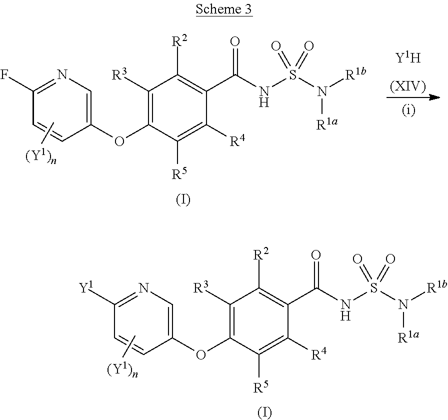

Compounds of formula (I) wherein Y¹ is as defined above may be prepared from the corresponding compounds of formula (I) wherein Y¹ is F according to process step (i) by displacement of the fluorine with compounds of formula (XIV) in the presence of a base. Suitable conditions for this interconversion of compounds of formula (I) comprise sodium hydride in THF at from room temperature to elevated temperatures.

According to a fourth process, compounds of formula (I) may be prepared by the process illustrated in Scheme 4.

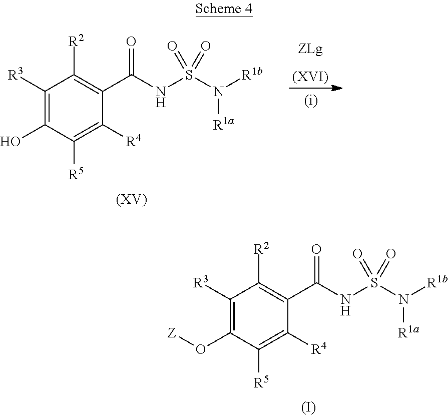

Compounds of formula (I) can be prepared from compounds of formula (XV) according to process step (i) by displacement of a suitable leaving group with compounds of formula (XVI) under SnAr reaction conditions as described for process step (ii) or (iii) in Scheme 1. Preferred conditions comprise cesium carbonate in DMSO at 70° C. for 18 hours.

Compounds of formulae (V), (VI), (VII), (VIII), (X), (XI), (XIII), (XIV), (XV) and (XVI) are either commercially available, known from the literature, easily prepared by methods well known to those skilled in the art, or can be made according to preparations described herein.

All new processes for preparing compounds of formula (I), and corresponding new intermediates employed in such processes, form further aspects of the present invention.

Compounds of the invention intended for pharmaceutical use may be administered as crystalline or amorphous products or may exist in a continuum of solid states ranging from fully amorphous to fully crystalline. They may be obtained, for example, as solid plugs, powders, or films by methods such as precipitation, crystallization, freeze drying, spray drying, or evaporative drying. Microwave or radio frequency drying may be used for this purpose.

They may be administered alone or in combination with one or more other compounds of the invention or in combination with one or more other drugs (or as any combination thereof). Generally, they will be administered as a formulation in association with one or more pharmaceutically acceptable excipients. The term ‘excipient’ is used herein to describe any ingredient other than the compound(s) of the invention. The choice of excipient will to a large extent depend on factors such as the particular mode of administration, the effect of the excipient on solubility and stability, and the nature of the dosage form.

In another aspect the invention provides a pharmaceutical composition comprising a compound of the invention together with one or more pharmaceutically acceptable excipients.

Pharmaceutical compositions suitable for the delivery of compounds of the present invention and methods for their preparation will be readily apparent to those skilled in the art. Such compositions and methods for their preparation may be found, for example, in “Remington's Pharmaceutical Sciences”, 19th Edition (Mack Publishing Company, 1995).

Suitable modes of administration include oral, parenteral, topical, inhaled/intranasal, rectal/intravaginal, and ocular/aural administration.

Formulations suitable for the aforementioned modes of administration may be formulated to be immediate and/or modified release. Modified release formulations include delayed-, sustained-, pulsed-, controlled-, targeted and programmed release.

The compounds of the invention may be administered orally. Oral administration may involve swallowing, so that the compound enters the gastrointestinal tract, or buccal or sublingual administration may be employed by which the compound enters the blood stream directly from the mouth. Formulations suitable for oral administration include solid formulations such as tablets, capsules containing particulates, liquids, or powders, lozenges (including liquid-filled), chews, multi- and nano-particulates, gels, solid solution, liposome, films, ovules, sprays, liquid formulations and buccal/mucoadhesive patches.

Liquid formulations include suspensions, solutions, syrups and elixirs. Such formulations may be employed as fillers in soft or hard capsules and typically comprise a carrier, for example, water, ethanol, polyethylene glycol, propylene glycol, methylcellulose, or a suitable oil, and one or more emulsifying agents and/or suspending agents. Liquid formulations may also be prepared by the reconstitution of a solid, for example, from a sachet.

The compounds of the invention may also be used in fast-dissolving, fast-disintegrating dosage forms such as those described in Expert Opinion in Therapeutic Patents, 11 (6), 981-986, by Liang and Chen (2001).

For tablet dosage forms, depending on dose, the drug may make up from 1 weight % to 80 weight % of the dosage form, more typically from 5 weight % to 60 weight % of the dosage form. In addition to the drug, tablets generally contain a disintegrant. Examples of disintegrants include sodium starch glycolate, sodium carboxymethyl cellulose, calcium carboxymethyl cellulose, croscarmellose sodium, crospovidone, polyvinylpyrrolidone, methyl cellulose, microcrystalline cellulose, lower alkyl-substituted hydroxypropyl cellulose, starch, pregelatinised starch and sodium alginate. Generally, the disintegrant will comprise from 1 weight % to 25 weight %, preferably from 5 weight % to 20 weight % of the dosage form.

Binders are generally used to impart cohesive qualities to a tablet formulation. Suitable binders include microcrystalline cellulose, gelatin, sugars, polyethylene glycol, natural and synthetic gums, polyvinylpyrrolidone, pregelatinised starch, hydroxypropyl cellulose and hydroxypropyl methylcellulose. Tablets may also contain diluents, such as lactose (monohydrate, spray-dried monohydrate, anhydrous and the like), mannitol, xylitol, dextrose, sucrose, sorbitol, microcrystalline cellulose, starch and dibasic calcium phosphate dihydrate.

Tablets may also optionally comprise surface active agents, such as sodium lauryl sulfate and polysorbate 80, and glidants such as silicon dioxide and talc. When present, surface active agents may comprise from 0.2 weight % to 5 weight % of the tablet, and glidants may comprise from 0.2 weight % to 1 weight % of the tablet.

Tablets also generally contain lubricants such as magnesium stearate, calcium stearate, zinc stearate, sodium stearyl fumarate, and mixtures of magnesium stearate with sodium lauryl sulphate. Lubricants generally comprise from 0.25 weight % to 10 weight %, preferably from 0.5 weight % to 3 weight % of the tablet. Other possible ingredients include anti-oxidants, colourants, flavouring agents, preservatives and taste-masking agents.

Exemplary tablets contain up to about 80% drug, from about 10 weight % to about 90 weight % binder, from about 0 weight % to about 85 weight % diluent, from about 2 weight % to about 10 weight % disintegrant, and from about 0.25 weight % to about 10 weight % lubricant. Tablet blends may be compressed directly or by roller to form tablets. Tablet blends or portions of blends may alternatively be wet-, dry-, or melt-granulated, melt congealed, or extruded before tabletting. The final formulation may comprise one or more layers and may be coated or uncoated; it may even be encapsulated. The formulation of tablets is discussed in “Pharmaceutical Dosage Forms: Tablets”, Vol. 1, by H. Lieberman and L. Lachman (Marcel Dekker, New York, 1980).

Suitable modified release formulations for the purposes of the invention are described in U.S. Pat. No. 6,106,864. Details of other suitable release technologies such as high energy dispersions and osmotic and coated particles are to be found in “Pharmaceutical Technology On-line”, 25(2), 1-14, by Verma et al (2001). The use of chewing gum to achieve controlled release is described in WO 00/35298.

The compounds of the invention may also be administered directly into the blood stream, into muscle, or into an internal organ. Suitable means for parenteral administration include intravenous, intraarterial, intraperitoneal, intrathecal, intraventricular, intraurethral, intrasternal, intracranial, intramuscular and subcutaneous. Suitable devices for parenteral administration include needle (including microneedle) injectors, needle-free injectors and infusion techniques.

Parenteral formulations are typically aqueous solutions which may contain excipients such as salts, carbohydrates and buffering agents (preferably to a pH of from 3 to 9), but, for some applications, they may be more suitably formulated as a sterile non-aqueous solution or as a dried form to be used in conjunction with a suitable vehicle such as sterile, pyrogen-free water.

The preparation of parenteral formulations under sterile conditions, for example, by lyophilisation, may readily be accomplished using standard pharmaceutical techniques well known to those skilled in the art.

The solubility of compounds of formula (I) used in the preparation of parenteral solutions may be increased by the use of appropriate formulation techniques, such as the incorporation of solubility-enhancing agents. Formulations for parenteral administration may be formulated to be immediate and/or modified release. Modified release formulations include delayed-, sustained-, pulsed-, controlled-, targeted and programmed release. Thus compounds of the invention may be formulated as a solid, semi-solid, or thixotropic liquid for administration as an implanted depot providing modified release of the active compound. Examples of such formulations include drug-coated stents and poly(dl-lactic-coglycolic)acid (PGLA) microspheres.

The compounds of the invention may also be administered topically to the skin or mucosa, that is, dermally or transdermally. Typical formulations for this purpose include gels, hydrogels, lotions, solutions, creams, ointments, dusting powders, dressings, foams, films, skin patches, wafers, implants, sponges, fibres, bandages and microemulsions. Liposomes may also be used. Typical carriers include alcohol, water, mineral oil, liquid petrolatum, white petrolatum, glycerin, polyethylene glycol and propylene glycol. Penetration enhancers may be incorporated—see, for example, J Pharm Sci, 88 (10), 955-958, by Finnin and Morgan (October 1999).

Other means of topical administration include delivery by electroporation, iontophoresis, phonophoresis, sonophoresis and microneedle or needle-free (e.g. Powderject™, Bioject™, etc.) injection.

The compounds of the invention can also be administered intranasally or by inhalation, typically in the form of a dry powder (either alone, as a mixture, for example, in a dry blend with lactose, or as a mixed component particle, for example, mixed with phospholipids, such as phosphatidylcholine) from a dry powder inhaler or as an aerosol spray from a pressurised container, pump, spray, atomiser (preferably an atomiser using electrohydrodynamics to produce a fine mist), or nebuliser, with or without the use of a suitable propellant, such as 1,1,1,2-tetrafluoroethane or 1,1,1,2,3,3,3-heptafluoropropane. For intranasal use, the powder may comprise a bioadhesive agent, for example, chitosan or cyclodextrin.

The pressurised container, pump, spray, atomizer, or nebuliser contains a solution or suspension of the compound(s) of the invention comprising, for example, ethanol, aqueous ethanol, or a suitable alternative agent for dispersing, solubilising, or extending release of the active, a propellant(s) as solvent and an optional surfactant, such as sorbitan trioleate, oleic acid, or an oligolactic acid.

Prior to use in a dry powder or suspension formulation, the drug product is micronised to a size suitable for delivery by inhalation (typically less than 5 microns). This may be achieved by any appropriate comminuting method, such as spiral jet milling, fluid bed jet milling, supercritical fluid processing to form nanoparticles, high pressure homogenisation, or spray drying.

Capsules (made, for example, from gelatin or hydroxypropylmethylcellulose), blisters and cartridges for use in an inhaler or insufflator may be formulated to contain a powder mix of the compound of the invention, a suitable powder base such as lactose or starch and a performance modifier such as l-leucine, mannitol, or magnesium stearate. The lactose may be anhydrous or in the form of the monohydrate, preferably the latter. Other suitable excipients include dextran, glucose, maltose, sorbitol, xylitol, fructose, sucrose and trehalose.

A suitable solution formulation for use in an atomiser using electrohydrodynamics to produce a fine mist may contain from 1 μg to 20 mg of the compound of the invention per actuation and the actuation volume may vary from 1 μl to 100 μl. A typical formulation may comprise a compound of formula (I), propylene glycol, sterile water, ethanol and sodium chloride. Alternative solvents which may be used instead of propylene glycol include glycerol and polyethylene glycol.

Suitable flavours, such as menthol and levomenthol, or sweeteners, such as saccharin or saccharin sodium, may be added to those formulations of the invention intended for inhaled/intranasal administration.

In the case of dry powder inhalers and aerosols, the dosage unit is determined by means of a valve which delivers a metered amount. Units in accordance with the invention are typically arranged to administer a metered dose or “puff” containing from 1 μg to 100 mg of the compound of formula (I). The overall daily dose will typically be in the range 1 μg to 200 mg which may be administered in a single dose or, more usually, as divided doses throughout the day.

The compounds of the invention may be administered rectally or vaginally, for example, in the form of a suppository, pessary, microbicide, vaginal ring or enema. Cocoa butter is a traditional suppository base, but various alternatives may be used as appropriate.

The compounds of the invention may also be administered directly to the eye or ear, typically in the form of drops of a micronised suspension or solution in isotonic, pH-adjusted, sterile saline. Other formulations suitable for ocular and aural administration include ointments, biodegradable (e.g. absorbable gel sponges, collagen) and non-biodegradable (e.g. silicone) implants, wafers, lenses and particulate or vesicular systems, such as niosomes or liposomes. A polymer such as crossed-linked polyacrylic acid, polyvinylalcohol, hyaluronic acid, a cellulosic polymer, for example, hydroxypropylmethylcellulose, hydroxyethylcellulose, or methyl cellulose, or a heteropolysaccharide polymer, for example, gelan gum, may be incorporated together with a preservative, such as benzalkonium chloride. Such formulations may also be delivered by iontophoresis.

The compounds of the invention may be combined with soluble macromolecular entities, such as cyclodextrin and suitable derivatives thereof or polyethylene glycol-containing polymers, in order to improve their solubility, dissolution rate, taste-masking, bioavailability and/or stability for use in any of the aforementioned modes of administration.

Drug-cyclodextrin complexes, for example, are found to be generally useful for most dosage forms and administration routes. Both inclusion and non-inclusion complexes may be used. As an alternative to direct complexation with the drug, the cyclodextrin may be used as an auxiliary additive, i.e. as a carrier, diluent, or solubiliser. Most commonly used for these purposes are alpha-, beta- and gamma-cyclodextrins, examples of which may be found in International Patent Applications Nos. WO 91/11172, WO 94/02518 and WO 98/55148.

For administration to human patients, the total daily dose of the compounds of the invention is typically in the range 1 μg to 10 g, such as 10 mg to 1 g, for example 25 mg to 500 mg depending, of course, on the mode of administration and efficacy. For example, oral administration may require a total daily dose of from 50 mg to 100 mg. The total daily dose may be administered in single or divided doses and may, at the physician's discretion, fall outside of the typical range given herein. These dosages are based on an average human subject having a weight of about 60 kg to 70 kg. The physician will readily be able to determine doses for subjects whose weight falls outside this range, such as infants and the elderly.

As noted above, the compounds of the invention are useful because they exhibit pharmacological activity in animals, i.e., Nav1.7 channel inhibition. More particularly, the compounds of the invention are of use in the treatment of disorders for which a Nav1.7 inhibitor is indicated. Preferably the animal is a mammal, more preferably a human.

In a further aspect of the invention there is provided a compound of the invention for use as a medicament.

In a further aspect of the invention there is provided a compound of the invention for the treatment of a disorder for which a Nav1.7 inhibitor is indicated.

In a further aspect of the invention there is provided use of a compound of the invention for the preparation of a medicament for the treatment of a disorder for which a Nav1.7 inhibitor is indicated.

In a further aspect of the invention there is provided a method of treating a disorder in an animal (preferably a mammal, more preferably a human) for which a Nav1.7 inhibitor is indicated, comprising administering to said animal a therapeutically effective amount of a compound of the invention.

Disorders for which a Nav1.7 inhibitor is indicated include pain, particularly neuropathic, nociceptive and inflammatory pain.

Physiological pain is an important protective mechanism designed to warn of danger from potentially injurious stimuli from the external environment. The system operates through a specific set of primary sensory neurones and is activated by noxious stimuli via peripheral transducing mechanisms (see Millan, 1999, Prog. Neurobiol., 57, 1-164 for a review). These sensory fibres are known as nociceptors and are characteristically small diameter axons with slow conduction velocities. Nociceptors encode the intensity, duration and quality of noxious stimulus and by virtue of their topographically organised projection to the spinal cord, the location of the stimulus. The nociceptors are found on nociceptive nerve fibres of which there are two main types, A-delta fibres (myelinated) and C fibres (non-myelinated). The activity generated by nociceptor input is transferred, after complex processing in the dorsal horn, either directly, or via brain stem relay nuclei, to the ventrobasal thalamus and then on to the cortex, where the sensation of pain is generated.

Pain may generally be classified as acute or chronic. Acute pain begins suddenly and is short-lived (usually twelve weeks or less). It is usually associated with a specific cause such as a specific injury and is often sharp and severe. It is the kind of pain that can occur after specific injuries resulting from surgery, dental work, a strain or a sprain. Acute pain does not generally result in any persistent psychological response. In contrast, chronic pain is long-term pain, typically persisting for more than three months and leading to significant psychological and emotional problems. Common examples of chronic pain are neuropathic pain (e.g. painful diabetic neuropathy, postherpetic neuralgia), carpal tunnel syndrome, back pain, headache, cancer pain, arthritic pain and chronic post-surgical pain.

When a substantial injury occurs to body tissue, via disease or trauma, the characteristics of nociceptor activation are altered and there is sensitisation in the periphery, locally around the injury and centrally where the nociceptors terminate. These effects lead to a hightened sensation of pain. In acute pain these mechanisms can be useful, in promoting protective behaviours which may better enable repair processes to take place. The normal expectation would be that sensitivity returns to normal once the injury has healed. However, in many chronic pain states, the hypersensitivity far outlasts the healing process and is often due to nervous system injury. This injury often leads to abnormalities in sensory nerve fibres associated with maladaptation and aberrant activity (Woolf & Salter, 2000, Science, 288, 1765-1768).

Clinical pain is present when discomfort and abnormal sensitivity feature among the patient's symptoms. Patients tend to be quite heterogeneous and may present with various pain symptoms. Such symptoms include: 1) spontaneous pain which may be dull, burning, or stabbing; 2) exaggerated pain responses to noxious stimuli (hyperalgesia); and 3) pain produced by normally innocuous stimuli (allodynia—Meyer et al., 1994, Textbook of Pain, 13-44). Although patients suffering from various forms of acute and chronic pain may have similar symptoms, the underlying mechanisms may be different and may, therefore, require different treatment strategies. Pain can also therefore be divided into a number of different subtypes according to differing pathophysiology, including nociceptive, inflammatory and neuropathic pain.

Nociceptive pain is induced by tissue injury or by intense stimuli with the potential to cause injury. Pain afferents are activated by transduction of stimuli by nociceptors at the site of injury and activate neurons in the spinal cord at the level of their termination. This is then relayed up the spinal tracts to the brain where pain is perceived (Meyer et al., 1994, Textbook of Pain, 13-44). The activation of nociceptors activates two types of afferent nerve fibres. Myelinated A-delta fibres transmit rapidly and are responsible for sharp and stabbing pain sensations, whilst unmyelinated C fibres transmit at a slower rate and convey a dull or aching pain. Moderate to severe acute nociceptive pain is a prominent feature of pain from central nervous system trauma, strains/sprains, burns, myocardial infarction and acute pancreatitis, post-operative pain (pain following any type of surgical procedure), posttraumatic pain, renal colic, cancer pain and back pain. Cancer pain may be chronic pain such as tumour related pain (e.g. bone pain, headache, facial pain or visceral pain) or pain associated with cancer therapy (e.g. postchemotherapy syndrome, chronic postsurgical pain syndrome or post radiation syndrome). Cancer pain may also occur in response to chemotherapy, immunotherapy, hormonal therapy or radiotherapy. Back pain may be due to herniated or ruptured intervertabral discs or abnormalities of the lumber facet joints, sacroiliac joints, paraspinal muscles or the posterior longitudinal ligament. Back pain may resolve naturally but in some patients, where it lasts over 12 weeks, it becomes a chronic condition which can be particularly debilitating.

Neuropathic pain is currently defined as pain initiated or caused by a primary lesion or dysfunction in the nervous system. Nerve damage can be caused by trauma and disease and thus the term ‘neuropathic pain’ encompasses many disorders with diverse aetiologies. These include, but are not limited to, peripheral neuropathy, diabetic neuropathy, post herpetic neuralgia, trigeminal neuralgia, back pain, cancer neuropathy, HIV neuropathy, phantom limb pain, carpal tunnel syndrome, central post-stroke pain and pain associated with chronic alcoholism, hypothyroidism, uremia, multiple sclerosis, spinal cord injury, Parkinson's disease, epilepsy and vitamin deficiency. Neuropathic pain is pathological as it has no protective role. It is often present well after the original cause has dissipated, commonly lasting for years, significantly decreasing a patient's quality of life (Woolf and Mannion, 1999, Lancet, 353, 1959-1964). The symptoms of neuropathic pain are difficult to treat, as they are often heterogeneous even between patients with the same disease (Woolf & Decosterd, 1999, Pain Supp., 6, S141-S147; Woolf and Mannion, 1999, Lancet, 353, 1959-1964). They include spontaneous pain, which can be continuous, and paroxysmal or abnormal evoked pain, such as hyperalgesia (increased sensitivity to a noxious stimulus) and allodynia (sensitivity to a normally innocuous stimulus).

The inflammatory process is a complex series of biochemical and cellular events, activated in response to tissue injury or the presence of foreign substances, which results in swelling and pain (Levine and Taiwo, 1994, Textbook of Pain, 45-56). Arthritic pain is the most common inflammatory pain. Rheumatoid disease is one of the commonest chronic inflammatory conditions in developed countries and rheumatoid arthritis is a common cause of disability. The exact aetiology of rheumatoid arthritis is unknown, but current hypotheses suggest that both genetic and microbiological factors may be important (Grennan & Jayson, 1994, Textbook of Pain, 397-407). It has been estimated that almost 16 million Americans have symptomatic osteoarthritis (OA) or degenerative joint disease, most of whom are over 60 years of age, and this is expected to increase to 40 million as the age of the population increases, making this a public health problem of enormous magnitude (Houge & Mersfelder, 2002, Ann Pharmacother., 36, 679-686; McCarthy et al., 1994, Textbook of Pain, 387-395). Most patients with osteoarthritis seek medical attention because of the associated pain. Arthritis has a significant impact on psychosocial and physical function and is known to be the leading cause of disability in later life. Ankylosing spondylitis is also a rheumatic disease that causes arthritis of the spine and sacroiliac joints. It varies from intermittent episodes of back pain that occur throughout life to a severe chronic disease that attacks the spine, peripheral joints and other body organs.

Another type of inflammatory pain is visceral pain which includes pain associated with inflammatory bowel disease (IBD). Visceral pain is pain associated with the viscera, which encompass the organs of the abdominal cavity. These organs include the sex organs, spleen and part of the digestive system. Pain associated with the viscera can be divided into digestive visceral pain and non-digestive visceral pain. Commonly encountered gastrointestinal (GI) disorders that cause pain include functional bowel disorder (FBD) and inflammatory bowel disease (IBD). These GI disorders include a wide range of disease states that are currently only moderately controlled, including, in respect of FBD, gastro-esophageal reflux, dyspepsia, irritable bowel syndrome (IBS) and functional abdominal pain syndrome (FAPS), and, in respect of IBD, Crohn's disease, ileitis and ulcerative colitis, all of which regularly produce visceral pain. Other types of visceral pain include the pain associated with dysmenorrhea, cystitis and pancreatitis and pelvic pain.

It should be noted that some types of pain have multiple aetiologies and thus can be classified in more than one area, e.g. back pain and cancer pain have both nociceptive and neuropathic components.

Other types of pain include:
pain resulting from musculo-skeletal disorders, including myalgia, fibromyalgia, spondylitis, sero-negative (non-rheumatoid) arthropathies, non-articular rheumatism, dystrophinopathy, glycogenolysis, polymyositis and pyomyositis;
heart and vascular pain, including pain caused by angina, myocardical infarction, mitral stenosis, pericarditis, Raynaud's phenomenon, scleredoma and skeletal muscle ischemia;
head pain, such as migraine (including migraine with aura and migraine without aura), cluster headache, tension-type headache mixed headache and headache associated with vascular disorders;
erythermalgia; and
orofacial pain, including dental pain, otic pain, burning mouth syndrome and temporomandibular myofascial pain.

A Nav1.7 inhibitor may be usefully combined with another pharmacologically active compound, or with two or more other pharmacologically active compounds, particularly in the treatment of pain. Such combinations offer the possibility of significant advantages, including patient compliance, ease of dosing and synergistic activity.

In the combinations that follow the compound of the invention may be administered simultaneously, sequentially or separately in combination with the other therapeutic agent or agents.

A Nav1.7 inhibitor of formula (I), or a pharmaceutically acceptable salt thereof, as defined above, may be administered in combination with one or more agents selected from:
an alternative Nav1.7 channel modulator, such as another compound of the present invention or a compound disclosed in WO 2009/012242 or WO 2010/079443;
an alternative sodium channel modulator, such as a Nav1.3 modulator (e.g. as disclosed in WO2008/118758); or a Nav1.8 modulator (e.g. as disclosed in WO 2008/135826, more particularly N-[6-Amino-5-(2-chloro-5-methoxyphenyl)pyridin-2-yl]-1-methyl-1H-pyrazole-5-carboxamide);
an inhibitor of nerve growth factor signaling, such as: an agent that binds to NGF and inhibits NGF biological activity and/or downstream pathway(s) mediated by NGF signaling (e.g. tanezumab), a TrkA antagonist or a p75 antagoinsist;
a compound which increases the levels of endocannabinoid, such as a compound with fatty acid amid hydrolase inhibitory (FAAH) activity, in particular those disclosed in WO 2008/047229 (e.g. N-pyridazin-3-yl-4-(3-{[5-(trifluoromethyl)pyridine-2-yl]oxy}benzylidene)piperidene-1-carboxamide);
an opioid analgesic, e.g. morphine, heroin, hydromorphone, oxymorphone, levorphanol, levallorphan, methadone, meperidine, fentanyl, cocaine, codeine, dihydrocodeine, oxycodone, hydrocodone, propoxyphene, nalmefene, nalorphine, naloxone, naltrexone, buprenorphine, butorphanol, nalbuphine or pentazocine;
a nonsteroidal antiinflammatory drug (NSAID), e.g. aspirin, diclofenac, diflusinal, etodolac, fenbufen, fenoprofen, flufenisal, flurbiprofen, ibuprofen, indomethacin, ketoprofen, ketorolac, meclofenamic acid, mefenamic acid, meloxicam, nabumetone, naproxen, nimesulide, nitroflurbiprofen, olsalazine, oxaprozin, phenylbutazone, piroxicam, sulfasalazine, sulindac, tolmetin or zomepirac;
a barbiturate sedative, e.g. amobarbital, aprobarbital, butabarbital, butabital, mephobarbital, metharbital, methohexital, pentobarbital, phenobartital, secobarbital, talbutal, theamylal or thiopental;
a benzodiazepine having a sedative action, e.g. chlordiazepoxide, clorazepate, diazepam, flurazepam, lorazepam, oxazepam, temazepam or triazolam;
an H₁ antagonist having a sedative action, e.g. diphenhydramine, pyrilamine, promethazine, chlorpheniramine or chlorcyclizine;
a sedative such as glutethimide, meprobamate, methaqualone or dichloralphenazone;
a skeletal muscle relaxant, e.g. baclofen, carisoprodol, chlorzoxazone, cyclobenzaprine, methocarbamol or orphrenadine;
an NMDA receptor antagonist, e.g. dextromethorphan ((+)-3-hydroxy-N-methylmorphinan) or its metabolite dextrorphan ((+)-3-hydroxy-N-methylmorphinan), ketamine, memantine, pyrroloquinoline quinine, cis-4-(phosphonomethyl)-2-piperidinecarboxylic acid, budipine, EN-3231 (MorphiDex®, a combination formulation of morphine and dextromethorphan), topiramate, neramexane or perzinfotel including an NR2B antagonist, e.g. ifenprodil, traxoprodil or (−)-(R)-6-{2-[4-(3-fluorophenyl)-4-hydroxy-1-piperidinyl]-1-hydroxyethyl-3,4-dihydro-2(1H)-quinolinone;
an alpha-adrenergic, e.g. doxazosin, tamsulosin, clonidine, guanfacine, dexmetatomidine, modafinil, or 4-amino-6,7-dimethoxy-2-(5-methane-sulfonamido-1,2,3,4-tetrahydroisoquinol-2-yl)-5-(2-pyridyl) quinazoline;
a tricyclic antidepressant, e.g. desipramine, imipramine, amitriptyline or nortriptyline;
an anticonvulsant, e.g. carbamazepine, lamotrigine, topiratmate or valproate;
a tachykinin (NK) antagonist, particularly an NK-3, NK-2 or NK-1 antagonist, e.g. (αR,9R)-7-[3,5-bis(trifluoromethyl)benzyl]-8,9,10,11-tetrahydro-9-methyl-5-(4-methylphenyl)-7H-[1,4]diazocino[2,1-g][1,7]-naphthyridine-6-13-dione (TAK-637), 5-[[(2R,3S)-2-[(1R)-1-[3,5-bis(trifluoromethyl)phenyl]ethoxy-3-(4-fluorophenyl)-4-morpholinyl]-methyl]-1,2-dihydro-3H-1,2,4-triazol-3-one (MK-869), aprepitant, lanepitant, dapitant or 3-[[2-methoxy-5-(trifluoromethoxy)phenyl]-methylamino]-2-phenylpiperidine (2S,3S);
a muscarinic antagonist, e.g oxybutynin, tolterodine, propiverine, tropsium chloride, darifenacin, solifenacin, temiverine and ipratropium;
a COX-2 selective inhibitor, e.g. celecoxib, rofecoxib, parecoxib, valdecoxib, deracoxib, etoricoxib, or lumiracoxib;
a coal-tar analgesic, in particular paracetamol;
a neuroleptic such as droperidol, chlorpromazine, haloperidol, perphenazine, thioridazine, mesoridazine, trifluoperazine, fluphenazine, clozapine, olanzapine, risperidone, ziprasidone, quetiapine, sertindole, aripiprazole, sonepiprazole, blonanserin, iloperidone, perospirone, raclopride, zotepine, bifeprunox, asenapine, lurasidone, amisulpride, balaperidone, palindore, eplivanserin, osanetant, rimonabant, meclinertant, Miraxion® or sarizotan;
a vanilloid receptor agonist (e.g. resinferatoxin) or antagonist (e.g. capsazepine);
a beta-adrenergic such as propranolol;
a local anaesthetic such as mexiletine;
a corticosteroid such as dexamethasone;
a 5-HT receptor agonist or antagonist, particularly a 5-HT_{1B/1D} agonist such as eletriptan, sumatriptan, naratriptan, zolmitriptan or rizatriptan;
a 5-HT_{2A} receptor antagonist such as R(+)-alpha-(2,3-dimethoxy-phenyl)-1-[2-(4-fluorophenylethyl)]-4-piperidinemethanol (MDL-100907);
a 5-HT₃ antagonist, such as ondansetron
a cholinergic (nicotinic) analgesic, such as ispronicline (TC-1734), (E)-N-methyl-4-(3-pyridinyl)-3-buten-1-amine (RJR-2403), (R)-5-(2-azetidinylmethoxy)-2-chloropyridine (ABT-594) or nicotine;
Tramadol®;
a PDEV inhibitor, such as 5[2-ethoxy-5-(4-methyl-1-piperazinyl-sulphonyl)phenyl]-1-methyl-3-n-propyl-1,6-dihydro-7H-pyrazolo[4,3-d]pyrimidin-7-one (sildenafil), (6R,12aR)-2,3,6,7,12,12a-hexahydro-2-methyl-6-(3,4-methylenedioxyphenyl)-pyrazino[2¹,1:6,1]-pyrido[3,4-b]indole-1,4-dione (IC-351 or tadalafil), 2[2-ethoxy-5-(4-ethyl-piperazin-1-yl-1-sulphonyl)-phenyl]-5-methyl-7-propyl-3H-imidazo[5,1-f][1,2,4]triazin-4-one (vardenafil), 5-(5-acetyl-2-butoxy-3-pyridinyl)-3-ethyl-2-(1-ethyl-3-azetidinyl)-2,6-dihydro-7H-pyrazolo[4,3-d]pyrimidin-7-one, 5-(5-acetyl-2-propoxy-3-pyridinyl)-3-ethyl-2-(1-isopropyl-3-azetidinyl)-2,6-dihydro-7H-pyrazolo[4,3-d]pyrimidin-7-one, 5-[2-ethoxy-5-(4-ethylpiperazin-1-ylsulphonyl)pyridin-3-yl]-3-ethyl-2-[2-methoxyethyl]-2,6-dihydro-7H-pyrazolo[4,3-d]pyrimidin-7-one, 4-[(3-chloro-4-methoxybenzyl)amino]-2-[(2S)-2-(hydroxymethyl)pyrrolidin-1-yl]-N-(pyrimidin-2-ylmethyl)pyrimidine-5-carboxamide, 3-(1-methyl-7-oxo-3-propyl-6,7-dihydro-1H-pyrazolo[4,3-d]pyrimidin-5-yl)-N-[2-(1-methylpyrrolidin-2-yl)ethyl]-4-propoxybenzenesulfonamide;
an alpha-2-delta ligand such as gabapentin, pregabalin, 3-methylgabapentin, (1α,3α,5α)(3-amino-methyl-bicyclo[3.2.0]hept-3-yl)-acetic acid, (3S,5R)-3-aminomethyl-5-methyl-heptanoic acid, (3S,5R)-3-amino-5-methyl-heptanoic acid, (3S,5R)-3-amino-5-methyl-octanoic acid, (2S,4S)-4-(3-chlorophenoxy)proline, (2S,4S)-4-(3-fluorobenzyl)-proline, [(1R,5R,6S)-6-(aminomethyl)bicyclo[3.2.0]hept-6-yl]acetic acid, 3-(1-aminomethyl-cyclohexylmethyl)-4H-[1,2,4]oxadiazol-5-one, C-[1-(1H-tetrazol-5-ylmethyl)-cycloheptyl]-methylamine, (3S,4S)-(1-aminomethyl-3,4-dimethyl-cyclopentyl)-acetic acid, (3S,5R)-3-aminomethyl-5-methyl-octanoic acid, (3S,5R)-3-amino-5-methyl-nonanoic acid, (3S,5R)-3-amino-5-methyl-octanoic acid, (3R,4R,5R)-3-amino-4,5-dimethyl-heptanoic acid and (3R,4R,5R)-3-amino-4,5-dimethyl-octanoic acid;
metabotropic glutamate subtype 1 receptor (mGluR1) antagonist;
a serotonin reuptake inhibitor such as sertraline, sertraline metabolite demethylsertraline, fluoxetine, norfluoxetine (fluoxetine desmethyl metabolite), fluvoxamine, paroxetine, citalopram, citalopram metabolite desmethylcitalopram, escitalopram, d,l-fenfluramine, femoxetine, ifoxetine, cyanodothiepin, litoxetine, dapoxetine, nefazodone, cericlamine and trazodone;
a noradrenaline (norepinephrine) reuptake inhibitor, such as maprotiline, lofepramine, mirtazepine, oxaprotiline, fezolamine, tomoxetine, mianserin, buproprion, buproprion metabolite hydroxybuproprion, nomifensine and viloxazine (Vivalan®), especially a selective noradrenaline reuptake inhibitor such as reboxetine, in particular (S,S)-reboxetine;
a dual serotonin-noradrenaline reuptake inhibitor, such as venlafaxine, venlafaxine metabolite O-desmethylvenlafaxine, clomipramine, clomipramine metabolite desmethylclomipramine, duloxetine, milnacipran and imipramine;
an inducible nitric oxide synthase (iNOS) inhibitor such as S-[2-[(1-iminoethyl)amino]ethyl]-L-homocysteine, S-[2-[(1-iminoethyl)-amino]ethyl]-4,4-dioxo-L-cysteine, S-[2-[(1-iminoethyl)amino]ethyl]-2-methyl-L-cysteine, (2S,5Z)-2-amino-2-methyl-7-[(1-iminoethyl)amino]-5-heptenoic acid, 2-[[(1R,3S)-3-amino-4-hydroxy-1-(5-thiazolyl)-butyl]thio]-5-chloro-3-pyridinecarbonitrile; 2-[[(1R,3S)-3-amino-4-hydroxy-1-(5-thiazolyl)butyl]thio]-4-chlorobenzonitrile, (2S,4R)-2-amino-4-[[2-chloro-5-(trifluoromethyl)phenyl]thio]-5-thiazolebutanol, 2-[[(1R,3S)-3-amino-4-hydroxy-1-(5-thiazolyl) butyl]thio]-6-(trifluoromethyl)-3pyridinecarbonitrile, 2-[[(1R,3S)-3-amino-4-hydroxy-1-(5-thiazolyl)butyl]thio]-5-chlorobenzonitrile, N-[4[2-(3-chlorobenzylamino)ethyl]phenyl]thiophene-2-carboxamidine, or guanidinoethyldisulfide;
an acetylcholinesterase inhibitor such as donepezil;
a prostaglandin E₂ subtype 4 (EP4) antagonist such as N-[({2-[4-(2-ethyl-4,6-dimethyl-1H-imidazo[4,5-c]pyridin-1-yl)phenyl]ethyl}amino)-carbonyl]-4-methylbenzenesulfonamide or 4-[(1S)-1-({[5-chloro-2-(3-fluorophenoxy)pyridin-3-yl]carbonyl}amino)ethyl]benzoic acid;
a microsomal prostaglandin E synthase type 1 (mPGES-1) inhibitor;
a leukotriene B4 antagonist; such as 1-(3-biphenyl-4-ylmethyl-4-hydroxy-chroman-7-yl)-cyclopentanecarboxylic acid (CP-105696), 5[2-(2-Carboxyethyl)-3-[6-(4-methoxyphenyl)-5E-hexenyl]oxyphenoxy]-valeric acid (ONO-4057) or DPC-11870;
a 5-lipoxygenase inhibitor, such as zileuton, 6-[(3-fluoro-5-[4-methoxy-3,4,5,6-tetrahydro-2H-pyran-4-yl])phenoxy-methyl]-1-methyl-2-quinolone (ZD-2138), or 2,3,5-trimethyl-6-(3-pyridylmethyl),1,4-benzoquinone (CV-6504).

There is also included within the scope the present invention combinations of a compound of the invention together with one or more additional therapeutic agents which slow down the rate of metabolism of the compound of the invention, thereby leading to increased exposure in patients. Increasing the exposure in such a manner is known as boosting. This has the benefit of increasing the efficacy of the compound of the invention or reducing the dose required to achieve the same efficacy as an unboosted dose. The metabolism of the compounds of the invention includes oxidative processes carried out by P450 (CYP450) enzymes, particularly CYP 3A4 and conjugation by UDP glucuronosyl transferase and sulphating enzymes. Thus, among the agents that may be used to increase the exposure of a patient to a compound of the present invention are those that can act as inhibitors of at least one isoform of the cytochrome P450 (CYP450) enzymes. The isoforms of CYP450 that may be beneficially inhibited include, but are not limited to, CYP1A2, CYP2D6, CYP2C9, CYP2C19 and CYP3A4. Suitable agents that may be used to inhibit CYP 3A4 include ritonavir, saquinavir, ketoconazole, N-(3,4-diluorobenzyl)-N-methyl-2-{[(4-methoxypyridin-3-yl)amino]sulfonyl}benzamide and N-(1-(2-(5-(4-fluorobenzyl)-3-(pyridin-4-yl)-1H-pyrazol-1-yl)acetyl)piperidin-4-yl)methanesulfonamide.

It is within the scope of the invention that two or more pharmaceutical compositions, at least one of which contains a compound of the invention, may conveniently be combined in the form of a kit suitable for coadministration of the compositions. Thus the kit of the invention comprises two or more separate pharmaceutical compositions, at least one of which contains a compound of the invention, and means for separately retaining said compositions, such as a container, divided bottle, or divided foil packet. An example of such a kit is the familiar blister pack used for the packaging of tablets, capsules and the like. The kit of the invention is particularly suitable for administering different dosage forms, for example, oral and parenteral, for administering the separate compositions at different dosage intervals, or for titrating the separate compositions against one another. To assist compliance, the kit typically comprises directions for administration and may be provided with a so-called memory aid.

In another aspect the invention provides a pharmaceutical product (such as in the form of a kit) comprising a compound of the invention together with one or more additional therapeutically active agents as a combined preparation for simultaneous, separate or sequential use in the treatment of a disorder for which a Nav1.7 inhibitor is indicated.

It is to be appreciated that all references herein to treatment include curative, palliative and prophylactic treatment.

In the non-limiting Examples and Preparations that are set out later in the description, and in the aforementioned Schemes, the following the abbreviations, definitions and analytical procedures may be referred to:

AcOH is acetic acid,

Cs₂CO₃ is caesium carbonate;

Cu(acac)₂ is copper (II) acetylacetonate;

CuI is copper (I) iodide;

Cu(OAc)₂ is copper (II) acetate;

DAD is diode array detector;

DCM is dichloromethane; methylene chloride;

DIPEA is N-ethyldiisopropylamine, N,N-diisopropylethylamine;

DMAP is 4-dimethylaminopyridine;

DMF is N,N-dimethylformamide;

DMSO is dimethyl sulphoxide;

EDCl is 1-(3-dimethylaminopropyl)-3-ethylcarbodiimide hydrochloride;

EDTA is ethylenediaminetetraacetic acid;

ELSD is evaporative light scattering detection;

Et₂O is diethyl ether;

EtOAc is ethyl acetate;

EtOH is ethanol;

HCl is hydrochloric acid;

IPA is isopropanol;

Ir₂(OMe)₂COD₂ is bis(1,5-cyclooctadiene)di-μ-methoxydiiridium (I);

K₂OC₃ is potassium carbonate;

KHSO₄ is potassium hydrogen sulphate;

KOAc is potassium acetate;

KOH is potassium hydroxide;

K₃PO₄ is potassium phosphate tribasic;

LCMS is liquid chromatography mass spectrometry (Rₜ=retention time)

LiOH is lithium hydroxide;

MeOH is methanol;

MgSO₄ is magnesium sulphate;

NaH is sodium hydride;

NaHCO₃ is sodium hydrogencarbonate;

Na₂CO₃ is sodium carbonate;

NaHSO₃ is sodium bisulphate;

NaHSO₄ is sodium hydrogensulphate;

NaOH is sodium hydroxide;

Na₂SO₄ is sodium sulphate;

NH₄Cl is ammonium chloride;

NMP is N-Methyl-2-pyrrolidone;

Pd/C is palladium on carbon;

Pd(PPh₃)₄ is palladium tetrakis;

Pd(dppf)₂Cl₂ is [1,1′-bis(diphenylphosphino)ferrocene]dichloropalladium(II), complex with dichloromethane;

THF is tetrahydrofuran;

THP is tetrahydropyran;

TLC is thin layer chromatography; and

WSCDI is 1-(3-dimethylaminopropyl)-3-ethylcarbodiimide hydrochloride.

¹H Nuclear magnetic resonance (NMR) spectra were in all cases consistent with the proposed structures. Characteristic chemical shifts (δ) are given in parts-per-million downfield from tetramethylsilane using conventional abbreviations for designation of major peaks: e.g. s, singlet; d, doublet; t, triplet; q, quartet; m, multiplet; br, broad. The following abbreviations have been used for common solvents: CDCl₃, deuterochloroform; d₆-DMSO, deuterodimethylsulphoxide; and CD₃OD, deuteromethanol.

Mass spectra, MS (m/z), were recorded using either electrospray ionisation (ESI) or atmospheric pressure chemical ionisation (APCI). When relevant, and unless stated otherwise, the m/z data provided are for isotopes ¹⁹F, ³⁵Cl and ⁷⁹Br.

Automated Preparative High Performance Liquid Chromatography (Auto-HPLC)

Certain compounds of the Examples and Preparations were purified using Automated Preparative High Performance Liquid Chromatography (HPLC). Reversed-phase HPLC conditions were either on Fraction Lynx systems or on a Trilution system.

In the case of the Fractionlynx system, Samples were submitted dissolved in 1 mL of DMSO. Depending on the nature of the compounds and the results of a pre-analysis, the purification was performed under either acidic (‘A-HPLC’), or basic (‘B-HPLC’) conditions at ambient temperature. A-HPLC was carried out on a Sunfire Prep C18 OBD column (19×100 mm, 5 μm). B-HPLC was carried out on an Xterra Prep MS C18 (19×100 mm, 5 μm), both from Waters. A flow rate of 18 mL/min was used with mobile phase A: water+0.1% modifier (v/v) and B: acetonitrile+0.1% modifier (v/v). For acidic runs the modifier was formic acid, for basic run the modifier was diethylamine. A Waters 2525 binary LC pump supplied a mobile phase with a composition of 5% B for 1 min then ran from 5% to 98% B over 6 min followed by a 2 min hold at 98% B.

Detection was achieved using a Waters 2487 dual wavelength absorbance detector set at 225 nm followed in series by a Polymer Labs PL-ELS 2100 detector and a Waters ZQ 2000 4 way MUX mass spectrometer in parallel. The PL 2100 ELSD was set at 30° C. with 1.6 L/min supply of Nitrogen. The Waters ZQ MS was tuned with the following parameters:

ES+ Cone voltage: 30 v Capillary: 3.20 kv

ES− Cone voltage: −30 v Capillary: −3.00 kv

Desolvation gas: 600 L/hr

Source Temp: 120° C.

Scan range 150-900 Da

The fraction collection was triggered by both MS and ELSD.

Quality control (QC) analysis was performed using a LCMS method. Acidic runs were carried out on a Sunfire C18 (4.6×50 mm, 5 μm), basic runs were carried out on a Xterra C18 (4.6×50 mm, 5 μm), both from Waters. A flow rate of 1.5 mL/min was used with mobile phase A: water+0.1% modifier (v/v) and B: acetonitrile+0.1% modifier (v/v). For acidic runs the modifier was formic acid, for basic run the modifier was ammonia. A Waters 1525 binary LC pump ran a gradient elution from 5% to 95% B over 3 min followed by a 1 min hold at 95% B. Detection was achieved using a Waters MUX UV 2488 detector set at 225 nm followed in series by a Polymer Labs PL-ELS 2100 detector and a Waters ZQ 2000 4 way MUX mass spectrometer in parallel. The PL 2100 ELSD was set at 30° C. with 1.6 L/min supply of Nitrogen. The Waters ZQ MS was tuned with the following parameters:

ES+ Cone voltage: 25 v Capillary: 3.30 kv

ES− Cone voltage: −30 v Capillary: −2.50 kv

Desolvation gas: 800 L/hr

Source Temp: 150° C.

Scan range 160-900 Da

Where the reversed-phase Trilution system was used the conditions were as follows:

Mobile phase A: 0.1% formic acid in water

Mobile phase B: 0.1% formic acid in acetonitrile

Column: Phenomenex C18 Luna 21.5 mm×15 cm with 5 micron particule size

Gradient: 95-5% A over 15 min, 15 min hold, 15 mL/min flow rate

UV: 200 nm-400 nm

Temperature: Room temperature

Liquid Chromatography Mass Spectrometry

Unless carried out by Auto-HPLC (under conditions of A-HPLC or B_HPLC) as described just above, or as specifically set out in the Examples and Preparations that follow, LCMS conditions were run according to one of the conditions given below (where ratios of solvents are given, the ratios are by volume):

Acidic 2 Minute LCMS

Mobile phase A: 0.1% formic acid in water

Mobile phase B: 0.1% formic acid in 70% methanol:30% isopropanol

Column: C18 phase Phenomenex 20×4.0 mm with 3 micron particle size

Gradient: 98-10% A over 1.5 min, 0.3 min hold, 0.2 re-equiilbration, 2 mL/min flow rate

UV: 210 nm-450 nm DAD

Temperature: 75° C.

Or

Mobile phase A: 0.1% formic acid in water

Mobile phase B: 0.1% formic acid in acetonitrile

Column: C18 phase Phenomenex 20×4.0 mm with 3 micron particle size

Gradient: 70-2% A over 1.5 min, 0.3 min hold, 0.2 re-equilibration, 1.8 mL/min flow rate

UV: 210nm-450nm DAD

Temperature: 75° C.

Acidic 4.5 Minute LCMS

Mobile phase A: 0.05% formic acid in water

Mobile phase B: acetonitrile

Column: Phenomenex Gemini C18 45×45 mm with 5 micron particle size

Gradient: 80-50% A over 0.5 min, 50-2% A over 3 min, 1 min hold, 0.2 min re-equilibration, 2.0 mL/min flow rate

UV: 220 nm-254 nm DAD

Temperature: 40° C.

Acidic 8 Minute LCMS

Mobile phase A: 0.05% formic acid in water

Mobile phase B: acetonitrile

Column: Phenomenex Gemini C18 45×45 mm with 5 micron particle size

Gradient: 80-50% A over 0.5 min, 50-2% A over 3 min, 4.5 min hold, 0.2 min re-equilibration, 2.0 mL/min flow rate

UV: 220 nm-254 nm DAD

Temperature: 40° C.

Acidic 6 Minute LCMS

Mobile phase A: 0.1% formic acid in water

Mobile phase B: 0.1% formic acid in acetonitrile

Column: C18 phase Waters Sunfire 50×4.6 mm with 5 micron particle size

Gradient: 95-5% A over 3 min, 1 min hold, 2 min re-equilibration, 1.5 mL/min flow rate

UV: 210 nm-450 nm DAD

Temperature: 50° C.

Basic 6 Minute LCMS

Mobile phase A: 0.1% ammonium hydroxide in water

Mobile phase B: 0.1% ammonium hydroxide in acetonitrile

Column: C18 phase Fortis 50×4.6 mm with 5micron particle size

Gradient: 95-5% A over 3 min, 1 min hold, 2 min re-equilibration, 1 mL/min flow rate

UV: 210 nm-450 nm DAD

Temperature: 50° C.

### EXAMPLE 1

### 4-[4-Chloro-3-(trifluoromethyl)phenoxy]-N-[(dimethylamino)sulfonyl]benzamide diethylamine salt

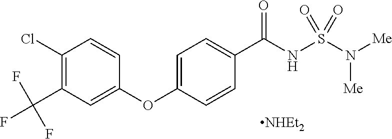 [SMILES: CN(C)S(=O)(=O)NC(=O)c1ccc(Oc2ccc(Cl)c(C(F)(F)F)c2)cc1 ; "·NHEt2"]

Method A

To a solution of 4-[4-chloro-3-(trifluoromethyl)phenoxy]benzoic acid, (Preparation 2, 45 mg, 0.14 mmol) in dichloromethane (2 mL) was added 4-dimethylaminopyridine (38 mg, 0.31 mmol), and 1-(3-dimethylaminopropyl)-3-ethylcarbodiimide:hydrochloride (60 mg, 0.31 mmol). The reaction mixture was stirred in a sealed Reactivial™, then N,N-dimethylsulfamide (Preparation 29, 31 mg, 0.25 mmol) was added. The reaction mixture was stirred at room temperature for 16 hours, then diluted with dichloromethane (5 mL), and 1 M aqueous hydrogen chloride solution (5 mL). The organic extracts were passed through a phase separation cartridge™, and evaporated in vacuo to afford a pale yellow solid (80 mg). The crude residues were dissolved in dimethylsulfoxide (50 mg/mL) and purified by B-HPLC to afford the title compound as the diethylamine salt (41.8 mg).

LCMS Rt=1.76 minutes MS m/z 422 [M−H]⁻

### EXAMPLE 2

### 4-[(5-Chloro-6-isobutoxvpvridin-3yl)oxy]-N-[(dimethylamino)sulfonyl]2,5-difluorobenzamide diethylamine salt

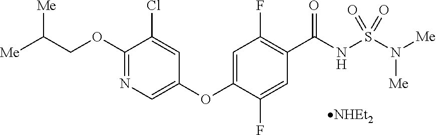 [SMILES: CC(C)COc1ncc(Oc2cc(F)c(C(=O)NS(=O)(=O)N(C)C)cc2F)cc1Cl ; "·NHEt2"]

Prepared according to Method A (Example 1) using 4-[(5-chloro-6-isobutoxypyridin-3-yl)oxy]-2,5-difluorobenzoic acid (Preparation 4) and N,N-dimethylsulfamide (Preparation 29). The title compound was isolated as the diethylamine salt.

LCMS Rt=1.89 minutes MS m/z 464 [MH]⁺

### EXAMPLE 3

### 4-[4-chloro-3-(trifluoromethyl)phenoxy]-N-[(methylamino)sulfonyl]benzamide diethylamine salt

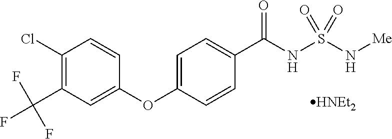 [SMILES: CNS(=O)(=O)NC(=O)c1ccc(Oc2ccc(Cl)c(C(F)(F)F)c2)cc1 ; "·HNEt2"]

Prepared according to Method A (Example 1) using 4-[4-chloro-3-(trifluoromethyl)phenoxy]benzoic acid (Preparation 2) and N-(4-methoxybenzyl)-N-methylsulfamide. After stirring at room temperature for 16 hours, TFA (0.5 mL) was added and the reaction stirred for a further 8 hours. The reaction was then quenched and purified according to Method A and the title compound was isolated as the diethylamine salt.

LCMS Rt=3.54 minutes MS m/z 407 [M−H]⁻

### EXAMPLE 4

### N-(aminosulfonyl)-4-(4-chloro-2-methoxyphenoxy)benzamide

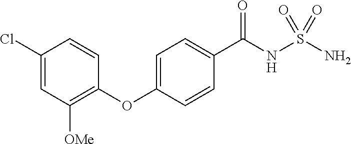 [SMILES: COc1cc(Cl)ccc1Oc1ccc(C(=O)NS(N)(=O)=O)cc1]

Prepared according to Method A (Example 1) using 4-(4-chloro-2-methoxyphenoxy)benzoic acid (Preparation 6) and sulfamide. The crude residue was purified using A-HPLC to afford the title compound.

LCMS Rt=1.49 minutes MS m/z 357 [M+H]⁺

### EXAMPLE 5

### 4-(4-chloro-2-methoxyphenoxy)-N-[(dimethylamino)sulfonyl]benzamide

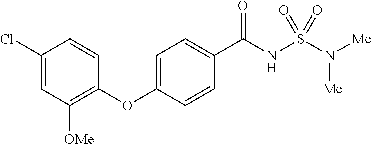 [SMILES: COc1cc(Cl)ccc1Oc1ccc(C(=O)NS(=O)(=O)N(C)C)cc1]

Prepared according to Method A (Example 1) using 4-(4-chloro-2-methoxyphenoxy)benzoic acid (Preparation 6) and N,N-dimethylsulfamide (Preparation 29). The crude residue was purified using A-HPLC to afford the title compound.

LCMS Rt=1.65 minutes MS m/z 383 [M−H]⁻

### EXAMPLE 6

### N-(aminosulfonyl)-4-(4-chloro-2-methoxvphenoxy)-2,5-difluorobenzamide

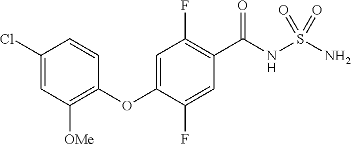 [SMILES: COc1cc(Cl)ccc1Oc1cc(F)c(C(=O)NS(N)(=O)=O)cc1F]

Prepared according to Method A (Example 1) using 4-(4-chloro-2-methoxyphenoxy)-2,5-difluorobenzoic acid (Preparation 10) and sulfamide. The crude residue was purified using A-HPLC to afford the title compound.

LCMS Rt=1.30 minutes MS m/z 393 [MH]⁺

### EXAMPLE 7

### 4-(4-chloro-2-methoxvphenoxy)-N-[(dimethylamino)sulfonyl]-2,5-difluorobenzamide

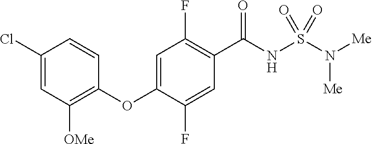 [SMILES: COc1cc(Cl)ccc1Oc1cc(F)c(C(=O)NS(=O)(=O)N(C)C)cc1F]

Prepared according to Method A (Example 1) using 4-(4-chloro-2-methoxyphenoxy)-2,5-difluorobenzoic acid (Preparation 10) and N,N-dimethylsulfamide (Preparation 29). The crude residue was purified using A-HPLC to afford the title compound.

LCMS Rt=1.40 minutes MS m/z 421 [MH]⁺

### EXAMPLE 8

### N-[(dimethylamino)sulfonyl]-4-(2-methoxyphenoxy)benzamide diethylamine salt

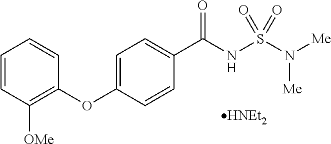 [SMILES: COc1ccccc1Oc1ccc(C(=O)NS(=O)(=O)N(C)C)cc1 ; "·HNEt2"]

Method B

To a solution of 4-(2-methoxyphenoxy)benzonitrile, (Preparation 12, 161 mg, 0.72 mmol) in ethylene glycol (3 mL) was added potassium hydroxide (500 mg, 8.8 mmol) followed by water (2 mL). The resulting mixture was heated at 120° C. with stirring for 6 hours. The mixture was cooled, diluted with dichloromethane (5 mL), and water (5 mL), and separated. The aqueous layer was acidified with 1M HCl, then washed with DCM (2×20 mL). The organic extracts were combined, passed through a phase separation cartridge™, and evaporated in vacuo to afford the corresponding carboxylic acid intermediate as a white solid (150 mg). To a solution of the carboxylic acid (150 mg) in dichloromethane (3 mL) was added 4-dimethylaminopyridine (175 mg, 1.43 mmol), and 1-(3-dimethylaminopropyl)-3-ethylcarbodiimide:hydrochloride (274 mg, 1.43 mmol). The reaction mixture was stirred in a sealed Reactivial™ until solubilised, then N,N-dimethylsulfamide (Preparation 29, 178 mg, 1.43 mmol) was added. The reaction mixture was stirred at 40° C. for 4 hours, then diluted with dichloromethane (5 mL), and 10% aqueous KHSO₄ solution (5 mL). The organic extracts were passed through a phase separation cartridge™, and evaporated in vacuo to afford a pale yellow solid (200 mg). Crude residues were dissolved in dimethylsulfoxide (50 mg/mL) and purified by B-HPLC to afford the title compound as the diethylamine salt (39 mg).

LCMS Rt=1.54 minutes MS m/z 351 [MH]⁺

### EXAMPLE 9

### N-[(dimethylamino)sulfonyl[-4-(3-methoxyphenoxy)benzamide diethylamine salt

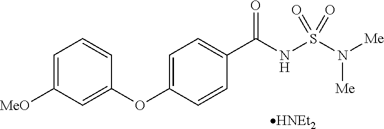 [SMILES: COc1cccc(Oc2ccc(C(=O)NS(=O)(=O)N(C)C)cc2)c1 ; "·HNEt2"]

Prepared according to Method B (Example 8) using 4-(3-methoxyphenoxy)benzonitrile (Preparation 13) and N,N-dimethylsulfamide (Preparation 29). The title compound was isolated as the diethylamine salt.

LCMS Rt=1.58 minutes MS m/z 351 [MH]⁺

### EXAMPLE 10

### N-(aminosulfonyl)-4-[(5-chloro-6-isobutoxypyridin-3-yl)oxy]benzamide diethylamine salt

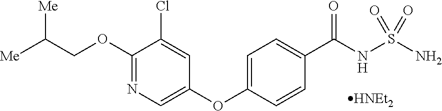 [SMILES: CC(C)COc1ncc(Oc2ccc(C(=O)NS(N)(=O)=O)cc2)cc1Cl ; "·HNEt2"]

Prepared according to Method B (Example 8) using 4-[(5-chloro-6-isobutoxypyridin-3-yl)oxy]benzonitrile (Preparation 14) and sulfamide. The title compound was isolated as the diethylamine salt.

LCMS Rt=1.72 minutes MS m/z 400 [MH]⁺

### EXAMPLE 11

### 4-[(5-chloro-6-isobutoxypyridin-3-yl)oxy]-N-[(dimethylamino)sulfonyl]benzamide diethylamine salt

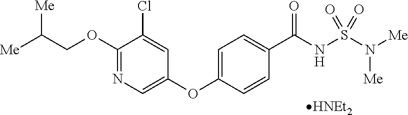 [SMILES: CC(C)COc1ncc(Oc2ccc(C(=O)NS(=O)(=O)N(C)C)cc2)cc1Cl ; "·HNEt2"]

Prepared according to Method B (Example 8) using 4-[(5-chloro-6-isobutoxypyridin-3-yl)oxy]benzonitrile (Preparation 14) and N,N-dimethylsulfamide (Preparation 29). The title compound was isolated as the diethylamine salt.

LCMS Rt=1.86 minutes MS m/z 428 [MH]⁺

### EXAMPLE 12

### 5-chloro-N-[(dimethylamino)sulfonyl]-2-fluoro-4-(2-methoxyphenoxy)benzamide diethylamine salt

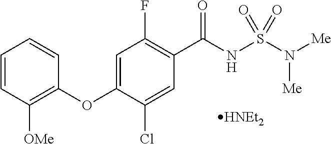 [SMILES: COc1ccccc1Oc1cc(F)c(C(=O)NS(=O)(=O)N(C)C)cc1Cl ; "·HNEt2"]

To a suspension of potassium carbonate (223 mg, 1.61 mmol) in dimethylsulfoxide (5 mL) was added 2-methoxyphenol (100 mg, 0.81 mmol). After stirring for 10 minutes at room temperature, 5-chloro-2,4-difluorobenzoic acid (155 mg, 0.81 mmol) was added and the resulting mixture heated to 100° C. for 24 hours with stirring. The mixture was then cooled and diluted with EtOAc (20 mL) then washed with 2M HCl (3×20 mL). The combined organic layers were dried over magnesium sulfate, filtered and concentrated in vacuo. The residue was purified by flash column chromatography eluting with 0-10% MeOH in DCM provide the corresponding carboxylic acid as a white solid (48 mg). To a solution of the carboxylic acid (48 mg) in dichloromethane (3 mL) was added 4-dimethylaminopyridine (99 mg, 0.81 mmol), and 1-(3-dimethylaminopropyl)-3-ethylcarbodiimide:hydrochloride (154 mg, 0.81 mmol). The reaction mixture was stirred in a sealed Reactivia™, then N,N-dimethylsulfamide (Preparation 29, 100 mg, 0.81 mmol) was added. The reaction mixture was stirred at room temperature, then diluted with dichloromethane (5 mL), and 10% aqueous KHSO₄ solution (5 mL). The organic extracts were passed through a phase separation cartridge™, and evaporated in vacuo to afford a solid (70 mg). The crude residue was dissolved in dimethylsulfoxide (50 mg/mL) and purified by B-HPLC to afford the title compound as the diethylamine salt (38 mg).

LCMS Rt=1.63 minutes MS m/z 403 [MH]⁺

### EXAMPLE 13

### N-[(dimethylamino)sulfonyl]-2,5-difluoro-4-(2-methoxyphenoxy)benzamide

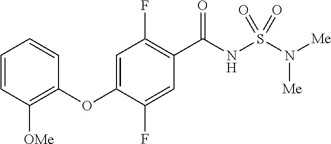 [SMILES: COc1ccccc1Oc1cc(F)c(C(=O)NS(=O)(=O)N(C)C)cc1F]

Method C

To a solution of methyl 2,5-difluoro-4-(2-methoxyphenoxy)benzoate (Preparation 15, 121 mg, 0.41 mmol) in methanol (1 mL) were added water (1 mL) and sodium hydroxide (100 mg, 2.50 mmol). The resulting mixture was heated to 55° C. with stirring for 5 hours. The mixture was cooled and diluted with EtOAc (20 mL), then washed with 2M HCl (3×20 mL). The combined organic layers were dried over magnesium sulfate, filtered and concentrated in vacuo to yield the corresponding carboxylic acid as a white solid (134 mg). To a solution of the carboxylic acid (60 mg) in dichloromethane (1 mL) was added 4-dimethylaminopyridine (66 mg, 0.52 mmol), and 1-(3-dimethylaminopropyl)-3-ethylcarbodiimide:hydrochloride (100 mg, 0.52 mmol). The reaction mixture was stirred in a sealed Reactivial™, then N,N-dimethylsulfamide (Preparation 29, 65 mg, 0.52 mmol) was added. The reaction mixture was stirred at room temperature, then diluted with dichloromethane (5 mL), and 10% aqueous KHSO₄ solution (5 mL). The organic extracts were passed through a phase separation cartridge™, and evaporated in vacuo to afford a solid (52 mg). The crude residue was dissolved in DMSO (50 mg/mL) and purified by A-HPLC to afford the title compound (28 mg).

LCMS Rt=1.33 minutes MS m/z 387 [MH]⁺

### EXAMPLE 14

### N-[(dimethylamino)sulfonyl]-2,5-difluoro-4-(3-methoxyphenoxy)benzamide diethylamine salt

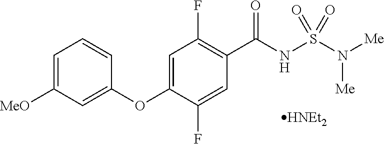 [SMILES: COc1cccc(Oc2cc(F)c(C(=O)NS(=O)(=O)N(C)C)cc2F)c1 ; "·HNEt2"]

Prepared according to Method C (Example 13) using methyl 2,5-difluoro-4-(3-methoxyphenoxy)benzoate (Preparation 16) and N,N-dimethylsulfamide (Preparation 29). The crude residue was purified using B-HPLC to afford the title compound as the diethylamine salt.

LCMS Rt=1.39 minutes MS m/z 387 [MH]⁺

### EXAMPLE 15

### N-(aminosulfonyl)-4-(4-chloro-2-pyridazin-4-ylphenoxy)benzamide

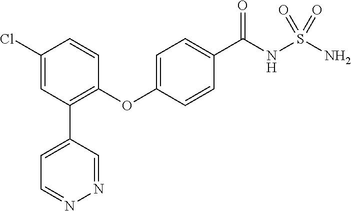 [SMILES: NS(=O)(=O)NC(=O)c1ccc(Oc2ccc(Cl)cc2-c2ccnnc2)cc1]

Prepared according to Method A (Example 1) using 4-(4-chloro-2-pyridazin-4-ylphenoxy)benzoic acid (Preparation 8) and sulfamide. The crude residue was purified using A-HPLC to afford the title compound.

LCMS Rt=1.32 minutes MS m/z 405 [MH]⁺

### EXAMPLE 16

### 4-(4-chloro-2-pyridazin-4-ylphenoxy)-N-[(dimethylamino)sulfonyl]benzamide

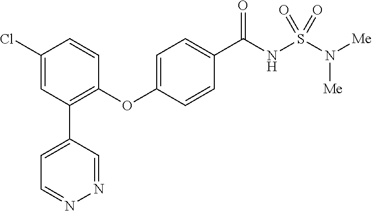 [SMILES: CN(C)S(=O)(=O)NC(=O)c1ccc(Oc2ccc(Cl)cc2-c2ccnnc2)cc1]

Prepared according to Method A (Example 1) using 4-(4-chloro-2-pyridazin-4-ylphenoxy)benzoic acid (Preparation 8) and N,N-dimethylsulfamide (Preparation 29). The crude residue was purified using A-HPLC to afford the title compound.

LCMS Rt=1.48 minutes MS m/z 433 [MH]⁺

### EXAMPLE 17

### 4-[(5-chloro-6-isopropoxypyridin-3-yl)oxy]-N-[(dimethylamino)sulfonyl]-2,5-difluorobenzamide-d7

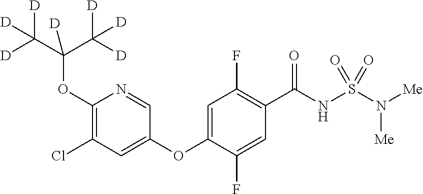 [SMILES: [2H]C([2H])([2H])C([2H])(Oc1ncc(Oc2cc(F)c(C(=O)NS(=O)(=O)N(C)C)cc2F)cc1Cl)C([2H])([2H])[2H]]

To a solution of 4-methylphenyl 4-[(5-chloro-6-isopropoxypyridin-3-yl)oxy]-2,5-difluorobenzoate-d7, (Preparation 17, 100 mg, 0.23 mmol) was added N,N-dimethylsulfamide (Preparation 29, 42 mg, 0.34 mmol). The resulting mixture was heated at 60° C. with stirring for 18 hours. The reaction mixture was cooled, then diluted with EtOAc (10 mL) and 10% aqueous citric acid (5 mL). The organic layers were separated, washed with water (2×5 mL), dried over magnesium sulfate, filtered and concentrated in vacuo to yield a white solid. The crude residues (55 mg) were dissolved in dimethylsulfoxide (50 mg/mL) and purified by A-HPLC to afford the title compound (26 mg).

LCMS Rt=1.58 minutes MS m/z 457 [MH]⁺

### EXAMPLE 18

### N-(aminosulfonyl)-4-(biphenyl-2-yloxy)-3-cyanobenzamide diethylamine salt

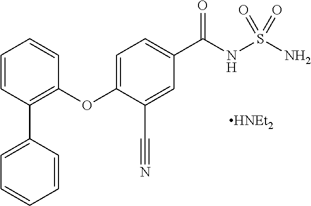 [SMILES: N#Cc1cc(C(=O)NS(N)(=O)=O)ccc1Oc1ccccc1-c1ccccc1 ; "·HNEt2"]

To a solution of 4-(biphenyl-2-yloxy)-3-cyanobenzoic acid (Preparation 18, 100 mg, 0.32 mmol) in DCM (0.2 mL) was added chlorosulfonylisocyanate (244 mg, 1.73 mmol). The resulting mixture was heated at 50° C. with stirring for 2 hours, then cooled and concentrated in vacuo. The residue was dissolved in acetonitrile (3 mL) and 1 mL of this solution was treated with an aqueous solution of ammonia (35%, 1 mL). The resulting solution was stirred for 2 hours before concentration in vacuo. The crude residues were dissolved in dimethylsulfoxide (50 mg/mL) and purified by B-HPLC to afford the title compound as the corresponding diethylamine salt (6 mg).

LCMS Rt=3.43 minutes MS m/z 394 [MH]⁺

### EXAMPLE 19

### 4-(biphenyl-2-yloxy)-3-cyano-N-[(ethylamino)sulfonyl]benzamide diethylamine salt

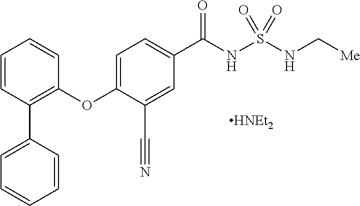 [SMILES: CCNS(=O)(=O)NC(=O)c1ccc(Oc2ccccc2-c2ccccc2)c(C#N)c1 ; "·HNEt2"]

To a solution of 4-(biphenyl-2-yloxy)-3-cyanobenzoic acid (Preparation 18, 100 mg, 0.32 mmol) in DCM (0.2 mL) was added chlorosulfonylisocyanate (244 mg, 1.73 mmol). The resulting mixture was heated at 50° C. with stirring for 2 hours, then cooled and concentrated in vacuo. The residue was dissolved in acetonitrile (3 mL) and 1 mL of this solution was treated with aqueous ethylamine (70%, 1 mL). The resulting solution was stirred for 2 hours before concentration in vacuo. The crude residues were dissolved in dimethylsulfoxide (50 mg/mL) and purified by B-HPLC to afford the title compound as the corresponding diethylamine salt (19 mg).

LCMS Rt=3.62 minutes MS m/z 422 [MH]⁺

The following Examples were prepared according to Library Protocol 1 using the appropriate amine of formula R⁷R⁸NH stated below:

### LIBRARY PROTOCOL 1 EXAMPLES 20-29

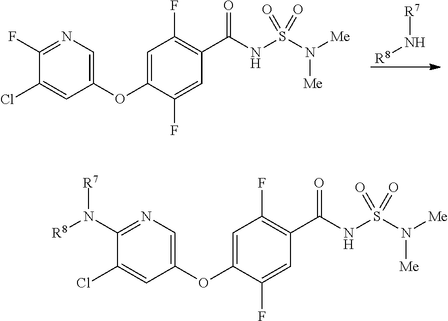

To amines of formula R⁷R⁸NH (wherein R⁷ and R⁸ are as previously defined for a compound of formula (I) unless otherwise stated, 0.105 mmol) was added a solution of 4-[(5-chloro-6-fluoropyridin-3-yl)oxy]-2,5-difluoro-N-[(dimethylamino)sulfonyl]-benzamide (Example 40, 28.6 mg, 0.075 mmol) in DMSO (0.6 mL), cesium pyridine (23 mg, 0.15 mmol) and DIPEA (39 μL, 0.225 mmol). The reaction mixtures were shaken in a sealed vial at 80° C. for 16 hours. The reaction mixtures were purified by HPLC (column DIKMA Diamonsil(2) C18 200 mm×20 mm×5 um or Boston Symmetrix ODS-H 150mm×30 mm×5 um, eluting with acetonitrile:water (containing 0.225% formic acid) gradient 10:90 to 85:15) to afford the title compounds as their formate salts.

| Ex. | Name | NR⁷R⁸ | MS m/z |
|---|---|---|---|
| 20 | 4-{[5-chloro-6-(3-fluoropyrrolidin-1-yl)pyridine-3- yl]oxy}-N-[(dimethylamino)sulfonyl]-2,5- difluorobenzamide formate salt | 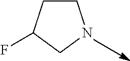 | 479 [MH]⁺ |
| 21 | 4-({5-chloro-6-[(cyclopropylmethyl)amino]pyridine-3- yl}oxy)-N-[(dimethylamino)sulfonyl]-2,5- difluorobenzamide formate salt | 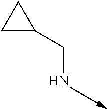 | 461 [MH]⁺ |
| 22 | 4-{[5-chloro-6-(dimethylamino)pyridine-3-yl]oxy}-N- [(dimethylamino)sulfonyl]-2,5-difluorobenzamide formate salt | 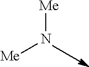 | 435 [MH]⁺ |
| 23 | 4-({5-chloro-6- [(cyclopropylmethyl)(methyl)amino]pyridine-3- yl}oxy)-N-[(dimethylamino)sulfonyl]-2,5- difluorobenzamide formate salt | 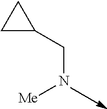 | 475 [MH]⁺ |
| 24 | 4-({5-chloro-6-[isopropyl(methyl)amino]pyridine-3- yl}oxy)-N-[(dimethylamino)sulfonyl]-2,5- difluorobenzamide formate salt | 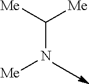 | 463 [MH]⁺ |
| 25 | 4-{[5-chloro-6-(methylamino)pyridine-3-yl]oxy}-N- [(dimethylamino)sulfonyl]-2,5-difluorobenzamide formate salt | 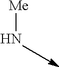 | 421 [MH]⁺ |
| 26 | 4-[(5-chloro-6-pyrrolidin-1-ylpyridin-3-yl)oxy]-N- [(dimethylamino)sulfonyl]-2,5-difluorobenzamide formate salt | 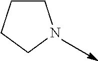 | 461 [MH]⁺ |
| 27 | 4-{[5-chloro-6-(cyclopropylamino)pyridin-3-yl]oxy}-N- [(dimethylamino)sulfonyl]-2,5-difluorobenzamide formate salt | 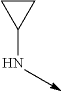 | 447 [MH]⁺ |
| 28 | 4-{[6-(pyridin[1.1.1]pent-1-ylamino)-5-chloropyridin- 3-yl]oxy}-N-[(dimethylamino)sulfonyl]-2,5- difluorobenzamide formate salt | 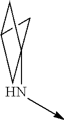 | 473 [MH]⁺ |
| 29 | 4-({5-chloro-6-[(2R)-2-methylpyrrolidin-1-yl]pyridine- 3-yl}oxy)-N-[(dimethylamino)sulfonyl]-2,5- dfluorobenzamide formate salt | 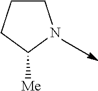 | 475 [MH]⁺ |

### EXAMPLE 30

### 4-[(5-chloro-6-isopropoxypyridin-3-yl)oxy]-N-[(dimethylamino)sulfonyl]-2,5-difluorobenzamide

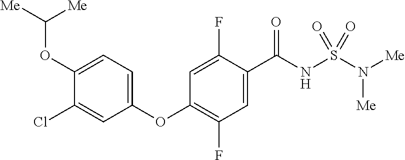 [SMILES: CC(C)Oc1ccc(Oc2cc(F)c(C(=O)NS(=O)(=O)N(C)C)cc2F)cc1Cl]

To a solution of 4-((5-chloro-6-isopropoxypyridin-3-yl)oxy)-2,5-difluorobenzamide (Preparation 25, 300 mg, 0.877 mmol) in anhydrous THF (10 mL) was added 1M NaHMDS (1.32 mL, 1.32 mmol) via syringe and the reaction stirred at room temperature for 1 hour. N,N-dimethylsulfamoyl chloride (189 mg, 1.32 mmol) was added and the reaction mixture warmed to 50° C. After 2 hours 1M LiHMDS (4 mL, 4 mmol) and dimethylsulfamoyl chloride (668 mg, 4.6 mmol) were added and the reaction stirred for 18 hours under nitrogen. The crude mixture was diluted with H₂O (50 mL) and acidified to approximately pH5 with acetic acid and the mixture was extracted with EtOAc (3×20 mL). The combined organic layers were washed with brine (20 mL), dried over MgSO₄, filtered and the solvent removed in vacuo to give a brown oil. The crude was purified by reverse phase chromatography eluting with 50:50:0.1 (H₂O:MeCN:HCOOH) to 0:100:0.1 to give the title compound (67 mg, 17%) as a beige solid.

¹H NMR (400 MHz, d₆-DMSO): δ ppm 1.28 (d, 6H), 2.85 (s, 6H), 5.25 (m, 1H), 7.19 (m, 1H), 7.72 (m, 2H), 7.97 (m, 1H), 8.09 (m, 1H), 11.87 (br s, 1H).

LCMS Rt=3.62 minutes MS m/z 448 [M−H]⁻

### EXAMPLE 31

### 4-[(5-chloro-6-isopropoxypyridin-3-yl)oxy]-N-[(methylamino)sulfonyl]-2,5-difluorobenzamide

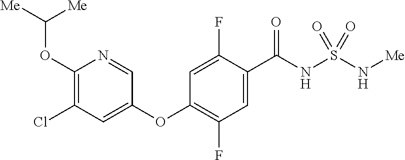 [SMILES: CNS(=O)(=O)NC(=O)c1cc(F)c(Oc2cnc(OC(C)C)c(Cl)c2)cc1F]

4-((5-chloro-6-isopropoxypyridin-3-yl)oxy)-2,5-difluoro-N-(N-(4-methoxybenzyl)-N-methylsulfamoyl)benzamide (Preparation 26, 16 mg, 0.029 mmol) was dissolved in 4M HCl in dioxane (5 mL, 20 mmol) and stirred at room temperature for 18 hours. The solvent was removed in vacuo and the residue taken up in neat TFA (5 mL). After stirring for 30 minutes the solvent was removed in vacuo and the residue purified by reverse phase chromatography eluting with 100:0:0.1 to 0:100:0.1 (H₂O:MeCN:HCO₂H) to give the title compound (11 mg, 88%) as a colourless solid.

¹H NMR (400 MHz, CD₃OD): δ ppm 1.39 (s, 6H), 2.70 (s, 3H), 5.35 (m, 1H), 6.88 (m, 1H), 7.62 (m, 1H), 7.69 (m, 1H), 7.95 (m, 1H).

LCMS Rt=3.47 minutes MS m/z 434 [M−H]⁻

### EXAMPLE 32

### 4-[(5-chloro-6-(cyclopropylmethoxy)pyridin-3-yl)oxy]-N-[(dimethylamino)sulfonyl]-2,5-difluorobenzamide

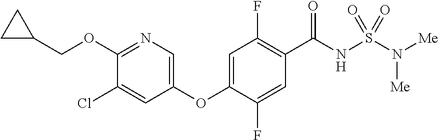 [SMILES: CN(C)S(=O)(=O)NC(=O)c1cc(F)c(Oc2cnc(OCC3CC3)c(Cl)c2)cc1F]

Method D

N-[(dimethylamino)sulfonyl]-2,4,5-trifluorobenzamide (Preparation 28, 100 mg, 0.35 mmol) was added to a pre-stirred solution of 5-chloro-6-(cyclopropylmethoxy)pyridin-3-ol (Preparation 34, 106 mg, 0.53 mmol) and potassium carbonate (147 mg, 1.06 mmol) in dimethyl sulfoxide (3 mL). The reaction was heated at 90° C. for 18 hours under nitrogen. The reaction was cooled to room temperature, diluted with water (30 mL) and extracted with ethyl acetate (3×30 mL). The combined organic layers were dried over magnesium sulphate, filtered and concentrated in vacuo. Preparative A-HPLC afforded the title compound (61 mg, 37%).

¹H NMR (400 MHz, CDCl₃) δ ppm 0.00 (m, 2H), 0.25 (m, 2H), 0.95 (m, 1H), 2.65 (s, 6H), 3.85 (d, 2H), 6.25 (dd, 1H), 7.10 (s, 1H), 7.50-7.55 (m, 2H), 8.25 (br.s, 1H).

LCMS Rt=3.59 minutes MS m/z mass ion not observed

### EXAMPLE 33

### 4-[(5-chloro-6-(2-fluoro-2-methylpropoxy)pyridin-3-yl)oxy]-N-[(dimethylamino)sulfonyl]-2,5-difluorobenzamide diethylamine salt

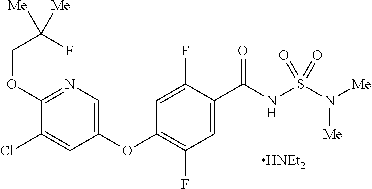 [SMILES: CN(C)S(=O)(=O)NC(=O)c1cc(F)c(Oc2cnc(OCC(C)(C)F)c(Cl)c2)cc1F ; "·HNEt2"]

Prepared according to Method D (Example 32) using 5-chloro-6-(2-fluoro-2-methylpropoxy)pyridin-3-ol (Preparation 35) and N-[(dimethylamino)sulfonyl]-2,4,5-trifluorobenzamide (Preparation 28). The crude residue was purified using B-HPLC to afford the title compound as the diethylamine salt.

LCMS Rt=3.48 minutes MS m/z 482 [MH]⁺

### EXAMPLE 34

### 4-[(5-chloro-6-(2,2,2-trifluoroethoxy)pyridin-3-yl)oxy]-N-[(dimethylamino)sulfonyl]-2,5-difluorobenzamide diethylamine salt

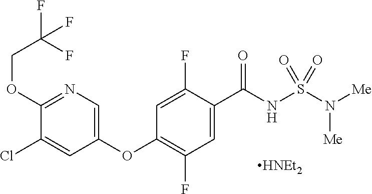 [SMILES: CN(C)S(=O)(=O)NC(=O)c1cc(F)c(Oc2cnc(OCC(F)(F)F)c(Cl)c2)cc1F ; "·HNEt2"]

Prepared according to Method D (Example 32) using 5-chloro-6-(2,2,2-trifluoroethoxy)pyridin-3-ol (Preparation 33) and N-[(dimethylamino)sulfonyl]-2,4,5-trifluorobenzamide (Preparation 28). The crude residue was purified using B-HPLC to afford the title compound as the diethylamine salt.

LCMS Rt=3.46 minutes MS m/z 490 [MH]⁺

### EXAMPLE 35

### 4-((5-chloro-4-(trifluoromethyl)pyridin-2-yl)oxy)-N-(N,N-dimethylsulfamoyl)-2,5-difluorobenzamide

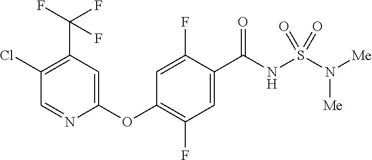 [SMILES: CN(C)S(=O)(=O)NC(=O)c1cc(F)c(Oc2cc(C(F)(F)F)c(Cl)cn2)cc1F]

Cesium carbonate (689 mg, 2.11 mmol) was added to a solution of N-[(dimethylamino)sulfonyl]-2,5-difluoro-4-hydroxybenzamide (Preparation 42, 237 mg, 0.85 mmol) and 2,5-dichloro-4-(trifluoromethyl)pyridine (183 mg, 0.85 mmol) in DMSO (5 mL). The reaction mixture was stirred at 70° C. for 18 hours. After cooling the reaction mixture was quenched with water (5 mL) and extracted with ethyl acetate (2×10 mL). The combined organic layers were dried over MgSO₄, filtered and concentrated in vacuo. The crude compound was purified using silica gel column chromatography eluting with dichloromethane:methanol (99:1 to 80:20) to give the title compound as a colourless solid (19 mg, 5%).

¹H NMR (400 MHz, MeOD): δ ppm 2.99 (s, 6H), 7.31-7.38 (m, 1H), 7.58-7.63 (m, 1H), 7.60 (s, 1H), 8.25 (s, 1H).

LCMS Rt=3.54 minutes MS m/z 458 [M−H]⁻

### EXAMPLE 36

### 4-((5-chloro-6-((1,1,1-trifluoropropan-2-yl)oxy)pyridin-3-yl)oxy)-N-[(dimethylamino)sulfonyl]-2,5-difluorobenzamide

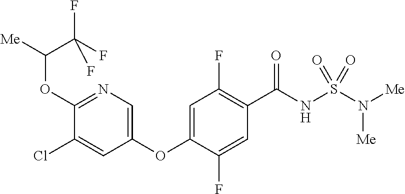 [SMILES: CC(Oc1ncc(Oc2cc(F)c(C(=O)NS(=O)(=O)N(C)C)cc2F)cc1Cl)C(F)(F)F]

To a 4-((5-chloro-6-fluoropyridin-3-yl)oxy)-N-(N,N-dimethylsulfamoyl)-2,5-difluorobenzamide solution (Example 40, 0.09 g, 0.22 mmol) in THF (2 mL) was added sodium hydride (0.026 g, 1.1 mmol) at room temperature, followed by 1,1,1-trifluoropropan-2-ol (0.2 mL, 2.2 mmol). The reaction mixture was stirred at 55° C. for 18 hours. After cooling, brine (20 mL) was added and the reaction mixture was extracted with ethyl acetate (2×100 mL). The combined organic layers were concentrated in vacuo and purified by A-HPLC to give the title compound (50 mg, 45%).

¹H NMR (400 MHz, d₆-DMSO): δ ppm 1.50 (d, 3H), 2.85 (s, 6H), 5.85 (m, 1H), 7.20 (s, 1H), 7.60 (s, 1H), 8.00 (s, 1H), 8.25 (s, 1H), 11.95 (s, 1H).

LCMS Rt=3.60 minutes MS m/z 502 [M−H]⁻

### EXAMPLE 37

### 4-((5-chloro-6-(1,1,1,3,3,3-hexafluoropropan-2-yl)oxy)pyridin-3-yl)oxy)-N-[dimethylamino)sulfonyl]-2,5-difluorobenzamide

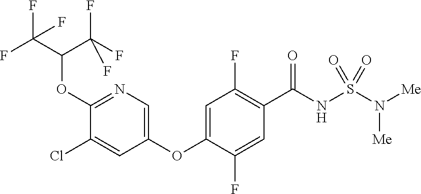 [SMILES: CN(C)S(=O)(=O)NC(=O)c1cc(F)c(Oc2cnc(OC(C(F)(F)F)C(F)(F)F)c(Cl)c2)cc1F]

To a 4-((5-chloro-6-fluoropyridin-3-yl)oxy)-N-(N,N-dimethylsulfamoyl)-2,5-difluorobenzamide solution (Example 40, 0.09 g, 0.22 mmol) in THF (3 mL) was added sodium hydride (0.026 g, 1.1 mmol) at room temperature, followed by 1,1,1,3,3,3-hexafluoropropan-2-ol (0.23 mL, 2.2 mmol). The reaction mixture was stirred at 80° C. for 18 hours, then for 100° C. for another 2 days. After cooling, brine (20 mL) was added and the reaction mixture was extracted with ethyl acetate (2×100 mL). The combined organic layers were concentrated in vacuo and purified by A-HPLC to give the title compound (36 mg, 30%).

¹H NMR (400 MHz, d₆-DMSO): δ ppm 2.80 (s, 6H), 6.82 (m, 1H), 7.15 (s, 1H), 7.35 (s, 1H), 7.75 (s, 1H), 8.20 (s, 1H), 11.90 (s, 1H).

LCMS Rt=3.72 minutes MS m/z 556 [M−H]⁻

### EXAMPLE 38

### N-(azetidin-1-ylsulfonyl)-4-(5-chloro-6-isopropoxypyridin-3-yl)oxy)-2,5-difluorobenzamide diethylamine salt

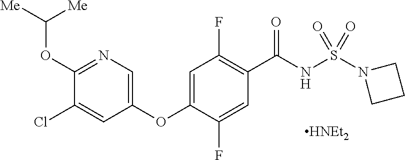 [SMILES: CC(C)Oc1ncc(Oc2cc(F)c(C(=O)NS(=O)(=O)N3CCC3)cc2F)cc1Cl ; "·HNEt2"]

Prepared according to Method A (Example 1) using 4-((5-chloro-6-isopropoxypyridin-3-yl)oxy)-2,5-difluorobenzoic acid (Preparation 44) and azetidine-1-sulfonamide (Preparation 46). The crude residue was purified using B-HPLC to afford the title compound as the diethylamine salt.

LCMS Rt=3.61 minutes MS m/z 460 [M−H]⁻

### EXAMPLE 39

### N-(aminosulfonyl)-4-[4-chloro-3-(trifluoromethyl)phenoxy]benzamide diethylamine salt

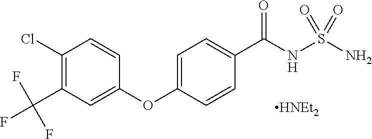 [SMILES: NS(=O)(=O)NC(=O)c1ccc(Oc2ccc(Cl)c(C(F)(F)F)c2)cc1 ; "·HNEt2"]

Prepared according to Method A (Example 1) using 4-[4-chloro-3-(trifluoromethyl)phenoxy]benzoic acid (Preparation 2) and sulfamide. The title compound was purified using B-HPLC and isolated as the diethylamine salt.

LCMS Rt=1.62 minutes MS m/z 393 [M−H]⁻

### EXAMPLE 40

### 4-[(5-chloro-6-fluoropyridin-3-yl)oxy]-N-[(dimethylamino)sulfonyl]-2,5-difluorobenzamide

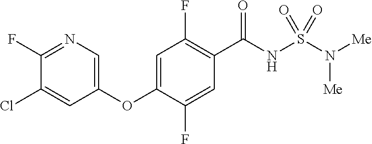 [SMILES: CN(C)S(=O)(=O)NC(=O)c1cc(F)c(Oc2cnc(F)c(Cl)c2)cc1F]

To a solution of 4-((5-chloro-6-fluoropyridin-3-yl)oxy)-2,5-difluorobenzoic acid (Preparation 19, 490 mg, 1.62 mmole) in DCM (100 mL) was added N,N-dimethylsulfamide (Preparation 29, 241 mg, 1.94 mmol), followed by addition of HOBt (2 mg, 120 umol), DMAP (237 mg, 1.94 mmol) and EDCl (311 mg, 1.62 mmol). The reaction mixture was stirred at 30 ⁰0 for 16 hours. The solvent was removed in vacuo then 2M HCl (20 mL) was added and extracted with EtOAc (3×15 mL). The combined organic layers were dried over sodium sulfate, filtered and concentrated in vacuo to afford the title compound (570 mg, 86%) that was used directly in library protocol 1.

¹H NMR (400 MHz, DMSO) δ ppm 2.85 (s, 6H), 7.35 (s, 1H), 7.75 (s, 1H), 8.20 (s, 1H), 8.25 (s, 1H), 11.95 (s, 1H).

LCMS Rt 2.82 min MS m/z 408 [M−H]⁻

### EXAMPLE 41

### N-(azetidin-1-ylsulfonyl)-5-chloro-4-{[5-chloro-6-(2,2,3,3-tetrafluoropropoxy)pyridin-3-yl]oxy}-2-fluorobenzamide

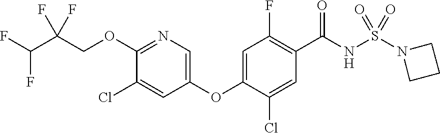 [SMILES: O=C(NS(=O)(=O)N1CCC1)c1cc(Cl)c(Oc2cnc(OCC(F)(F)C(F)F)c(Cl)c2)cc1F]

N,N-Diisopropylethylamine (0.50 mL, 3.01 mmol), 4-dimethylaminopyridine (0.28 g, 2.26 mmol) and 1-ethyl-3-(3-dimethylaminopropyl)carbodiimide (0.43 g, 2.26 mmol) were added to a suspension of 5-chloro-4-((5-chloro-6-(2,2,3,3-tetrafluoropropoxy)pyridin-3-yl)oxy)-2-fluorobenzoic acid (WO2012007861, 0.65 g, 1.50 mmol) in CH₂Cl₂ (10 mL). The mixture was stirred for 15 mins then azetidine-1-sulfonamide (Preparation 46, 0.31 g, 2.26 mmol) was added and the reaction mixture was stirred overnight at room temperature. The reaction mixture was diluted with ethyl acetate (100 mL), washed with water (100 mL), the organic layer dried over MgSO₄ and the filtrate evaporated under reduced pressure. The crude product was purified by reverse phase chromatography (MeCN/H₂O with 0.1% NH₄OH) to give the title compound as a solid (0.44 g, 53%).

¹H NMR (400 MHz, CDCl₃): δ 2.29 (quin, 2H), 4.25 (t, 4H), 4.79 (t, 2H), 6.22-5.93 (m, 1H), 6.60 (d, 1H), 7.54 (d, 1H), 7.93 (d, 1H), 8.25 (d, 1H) ppm.

¹⁹F NMR (376 MHz, CDCl₃): δ −138.76 (s), −124.73 (s), −108.85 (s) ppm.

LCMS Rt=2.92 minutes MS m/z 550 [MH]⁺

### EXAMPLE 42

### N-(azetidin-1-ylsulfonyl)-5-chloro-4-(3,4-dichlorophenoxy)-2-fluorobenzamide

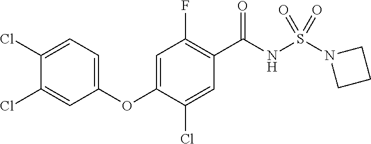 [SMILES: O=C(NS(=O)(=O)N1CCC1)c1cc(Cl)c(Oc2ccc(Cl)c(Cl)c2)cc1F]

N,N-Diisopropylethylamine (0.50 mL, 3.01 mmol), 4-dimethylaminopyridine (0.28 g, 2.26 mmol) and 1-ethyl-3-(3-dimethylaminopropyl)carbodiimide (0.43 g, 2.26 mmol) were added to a suspension of 5-chloro-4-(3,4-dichlorophenoxy)-2-fluorobenzoic acid (Preparation 50, 0.51 g, 1.50 mmol) in CH₂Cl₂ (10 mL). The mixture was stirred for 15 min and azetidine-1-sulfonamide (Preparation 46, 0.31 g, 2.26 mmol) was added and reaction was stirred overnight at room temperature. The reaction mixture was diluted with EtOAc (100 mL), washed with water (100 mL), organic layer was dried over MgSO₄ and filtrate was evaporated under reduced pressure. The crude product was purified by reverse phase (MeCN/H₂O with 0.1% NH₄OH) to give the title compound as a white solid (0.32 g, 46% yield).

¹H NMR (400 MHz, CDCl₃): δ 2.29 (quin, 2H), 4.26 (t, 4H), 6.67 (d, 1H), 6.94 (d, 1H), 7.20 (s, 1H), 7.53 (d, 1H), 8.25 (d, 1H) ppm.

¹⁹F NMR (376 MHz, CDCl₃): δ −109.12 (s) ppm.

LCMS Rt=2.87 minutes MS m/z 453 [MH]⁺

The compounds of formula (I) that follow may be prepared by procedures analgous to those described in the aforementioned Schemes, foregoing Examples 1-42 and the corresponding preparations, or by processes similar to either.

4-{[5-Chloro-6-(2,2,3,3,3-pentafluoropropoxy)pyridin-3-yl]oxy}-N-[(dimethylamino)sulfonyl]-2,5-difluorobenzamide;

5-chloro-4-[3-chloro-4-(trifluoromethyl)phenoxy]-N-[(dimethylamino)sulfonyl]-2-fluorobenzamide;

5-chloro-4-[3-chloro-4-(trifluoromethyl)phenoxy]-N-[(3,3-difluoroazetidin-1-yl)sulfonyl]-2-fluorobenzamide;

N-(azetidin-1-ylsulfonyl)-5-chloro-4-[3-chloro-4-(trifluoromethyl)phenoxy]-2-fluorobenzamide;

5-chloro-4-[4-chloro-3-(trifluoromethyl)phenoxy]-N-[(dimethylamino)sulfonyl]-2-fluorobenzamide;

5-chloro-4-[4-chloro-3-(trifluoromethyl)phenoxy]-N-[(3,3-difluoroazetidin-1-yl)sulfonyl]-2-fluorobenzamide;

5-chloro-4-[3-chloro-4-(trifluoromethoxy)phenoxy]-N-[(dimethylamino)sulfonyl]-2-fluorobenzamide;

5-chloro-4-[3-chloro-4-(trifluoromethoxy)phenoxy]-N-[(3,3-difluoroazetidin-1-yl)sulfonyl]-2-fluorobenzamide;

5-chloro-4-[4-chloro-3-(trifluoromethoxy)phenoxy]-N-[(dimethylamino)sulfonyl]-2-fluorobenzamide;

N-(azetidin-1-ylsulfonyl)-5-chloro-4-[4-chloro-3-(trifluoromethyl)phenoxy]-2-fluorobenzamide;

5-chloro-4-[4-chloro-3-(trifluoromethoxy)phenoxy]-N-[(3,3-difluoroazetidin-1-yl)sulfonyl]-2-fluorobenzamide;

N-(azetidin-1-ylsulfonyl)-5-chloro-4-[3-chloro-4-(trifluoromethoxy)phenoxy]-2-fluorobenzamide;

5-chloro-4-(3,4-dichlorophenoxy)-N-[(dimethylamino)sulfonyl]-2-fluorobenzamide;

5-chloro-4-(3,4-dichlorophenoxy)-N-[(3,3-difluoroazetidin-1-yl)sulfonyl]-2-fluorobenzamide;

N-(azetidin-1-ylsulfonyl)-5-chloro-4-[4-chloro-3-(trifluoromethoxy)phenoxy]-2-fluorobenzamide;

5-chloro-4-{[5-chloro-6-(2,2,2-trifluoro-1,1-dimethylethoxy)pyridin-3-yl]oxy}-N-[dimethylamino)sulfonyl]-2-fluorobenzamide;

5-chloro-4-{[5-chloro-6-(2,2,3,3-tetrafluoropropoxy)pyridin-3-yl]oxy}-N-[(dimethylamino)sulfonyl]-2-fluorobenzamide;

N-(azetidin-1-ylsulfonyl)-5-chloro-4-{[5-chloro-6-(2,2,2-trifluoro-1,1-dimethylethoxy)pyridin-3-yl]oxy}-2-fluorobenzamide;

5-chloro-4-{[5-chloro-6-(2,2,3,3,3-pentafluoropropoxy)pyridin-3-yl]oxy}-N-[(dimethylamino)sulfonyl]-2-fluorobenzamide;

5-chloro-4-{[5-chloro-6-(2,2,2-trifluoroethoxy)pyridin-3-yl]oxy}-N-[(dimethylamino)sulfonyl]-2-fluorobenzamide;

5-chloro-4-{[5-chloro-6-(2,2,3,3,3-pentafluoropropoxy)pyridin-3-yl]oxy}-N-[(3,3-difluoroazetidin-1-yl)sulfonyl]-2-fluorobenzamide;

N-(azetidin-1-ylsulfonyl)-5-chloro-4-{[5-chloro-6-(2,2,2-trifluoroethoxy)pyridin-3-yl]oxy}-2-fluorobenzamide;

5-chloro-4-{[5-chloro-6-(2,2,2-trifluoro-1,1-dimethylethoxy)pyridin-3-yl]oxy}-N-[(3,3-difluoroazetidin-1-yl)sulfonyl]-2-fluorobenzamide;

N-(azetidin-1-ylsulfonyl)-5-chloro-4-{[5-chloro-6-(2,2,3,3,3-pentafluoropropoxy)pyridin-3-yl]oxy}-2-fluorobenzamide;

5-chloro-4-{[5-chloro-6-(2,2,3,3-tetrafluoropropoxy)pyridin-3-yl]oxy}-N-[(3,3-difluoroazetidin-1-yl)sulfonyl]-2-fluorobenzamide;

5-chloro-4-{[5-chloro-6-(2,2,2-trifluoroethoxy)pyridin-3-yl]oxy}-N-[(3,3-difluoroazetidin-1-yl)sulfonyl]-2-fluorobenzamide;

N-(azetidin-1-ylsulfonyl)-4-{[5-chloro-6-(2,2,3,3,3-pentafluoropropoxy)pyridin-3-yl]oxy}-2,5-difluorobenzamide;

4-{[5-chloro-6-(2,2,3,3,3-pentafluoropropoxy)pyridin-3-yl]oxy}-N-[(3,3-difluoroazetidin-1-yl)sulfonyl]-2,5-difluorobenzamide;

5-chloro-4-{[5-chloro-6-(2,2,2-trifluoro-1-methylethoxy)pyridin-3-yl]oxy}-N-[(dimethylamino)sulfonyl]-2-fluorobenzamide;

5-chloro-4-{[5-chloro-6-(2,2,2-trifluoro-1-methylethoxy)pyridin-3-yl]oxy}-N-[(3,3-difluoroazetidin-1-yl)sulfonyl]-2-fluorobenzamide; and

N-(azetidin-1-ylsulfonyl)-5-chloro-4-{[5-chloro-6-(2,2,2-trifluoro-1-methylethoxy)pyridin-3-yl]oxy}-2-fluorobenzamide;

### Preparation 1

### 4-[4-chloro-3-(trifluoromethyl)phenoxy]benzonitrile

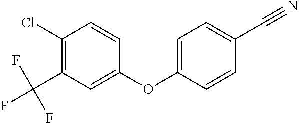 [SMILES: N#Cc1ccc(Oc2ccc(Cl)c(C(F)(F)F)c2)cc1]

Method E

To a solution of 3-chloro-4-(trifluoromethyl)phenol (200 mg, 1.02 mmol) in dimethylsulfoxide (7 mL) was added potassium carbonate (281 mg, 2.04 mmol) followed by 4-fluorobenzonitrile (123 mg, 1.02 mmol). The reaction mixture was stirred at 110° C. for 48 hours. The reaction mixture was cooled to room temperature and diluted with ethyl acetate (25 mL) and water (25 mL). The organic phase was separated and washed with water (2×15 mL), dried over anhydrous magnesium sulfate, filtered and evaporated in vacuo to obtain the title compound as a colourless gum (335 mg) which was used in the next step without further purification.

¹H NMR (400 MHz, CDCl₃): δ ppm 7.01-7.07 (m, 2H), 7.15-7.19 (m, 1H), 7.39 (d, 1H), 7.53 (d, 1H), 7.64-7.69 (m, 2H).

LCMS Rt=1.80 minutes MS m/z mass ion not observed

### Preparation 2

### 4-[4-Chloro-3-(trifluoromethyl)phenoxy]benzoic acid

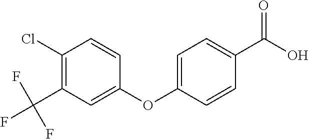 [SMILES: O=C(O)c1ccc(Oc2ccc(Cl)c(C(F)(F)F)c2)cc1]

Method F

To a solution of 4-[4-chloro-3-(trifluoromethyl)phenoxy]benzonitrile (Preparation 1, 335 mg, 1.12 mmol) in ethanol (5 mL) and water (5 mL) was added potassium hydroxide flakes (500 mg, 8.8 mmol). The resulting mixture was stirred under nitrogen at reflux for 16 hours and then evaporated in vacuo. The residue was dissolved in ethyl acetate (30 mL) and washed with 2M aqueous hydrogen chloride solution (10 mL). The organic extracts were dried over anhydrous magnesium sulphate and evaporated in vacuo to afford the title compound as a white solid (314 mg, 88%) which was used in the next step without further purification.

¹HNMR (400 MHz, d₆-DMSO): δ 7.11-7.17 (m, 2H), 7.41 (dd, 1H), 7.58 (d, 1H), 7.76 (d, 1H), 7.93-8.00 (m, 2H).

LCMS Rt=1.74 minutes MS m/z 315 [M−H]⁻

### Preparation 3

### Methyl 4-[(5-chloro-6-isobutoxypyridin-3-yl)oxy]-2,5-difluorobenzoate

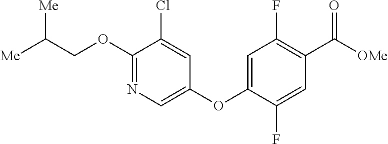 [SMILES: COC(=O)c1cc(F)c(Oc2cnc(OCC(C)C)c(Cl)c2)cc1F]

To a solution of 5-chloro-6-isobutoxypyridin-3-ol (Preparation 5, 100 mg, 0.496 mmol) in 5 mL of dimethylsulfoxide was added potassium carbonate (137 mg, 0.992 mmol) followed by methyl 2,4,5-trifluorobenzoate (94.3 mg, 0.496 mmol). The reaction mixture was stirred at 50° C. for 16 hours and then cooled to room temperature and diluted with ethyl acetate (25 mL) and water (20 mL). The organic phase was separated and washed with water (2×20 mL), dried over anhydrous magnesium sulfate, filtered and evaporated in vacuo to afford the title compound as a colourless gum (160 mg) which was used in the next step with no further purification.

¹H NMR (400 MHz, d₆-DMSO): δ ppm 0.98 (d, 6H), 2.05 (m, 1H), 3.83 (s, 3H), 4.10 (d, 2H), 7.11 (m, 1H), 7.82 (m, 1H), 8.02 (d, 1H), 8.10 (d, 1H).

LCMS Rt=1.72 minutes MS m/z 372 [MH]⁺

### Preparation 4

### 4-[(5-Chloro-6-isobutoxypyridin-3-yl)oxy]-2,5-difluorobenzoic acid

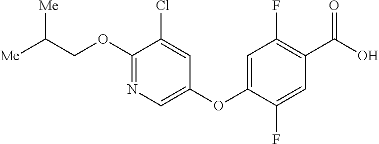 [SMILES: CC(C)COc1ncc(Oc2cc(F)c(C(=O)O)cc2F)cc1Cl]

Method G

To a solution of methyl 4-[(5-chloro-6-isobutoxypyridin-3-yl)oxy]-2,5-difluorobenzoate (Preparation 3, 160 mg , 0.43 mmol) in methanol (2 mL) was added 2 M aqueous sodium hydroxide solution (1.5 mL, 3.0 mmol) and the reaction mixture stirred under nitrogen at 55° C. for 16 hours. The solvents were evaporated in vacuo and the residue diluted with ethyl acetate (30 mL) and 1 M aqueous hydrogen chloride solution (10 mL). The organic extract was dried over anhydrous magnesium sulphate and evaporated in vacuo to afford the title compound as a white solid (163 mg, 100%).

¹H NMR (400 MHz, d₆-DMSO): δ ppm 0.98 (d, 6H), 2.00-2.12 (m, 1H), 4.10 (d, 2H), 7.02-7.11 (m, 1H), 7.74-7.82 (m, 1H), 8.00 (d, 1H), 8.09 (d, 1H).

LCMS Rt=1.85 minutes MS m/z 356 [M−H]⁻

### Preparation 5

### 5-Chloro-6-isobutoxypyridin-3-ol

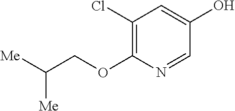 [SMILES: CC(C)COc1ncc(O)cc1Cl]

To a suspension of 5-chloro-6-isobutoxypyridin-3-ylboronic acid (3.02 g, 13.1 mmol) in acetic acid/water (1:1, 20 mL) cooled to 0° C., was slowly added peracetic acid (3.9 mL, 20.0 mmol) and the reaction mixture was maintained at 0° C. for 1.5 hours and then at room temperature for 1 hour. Additional peracetic acid (3.9 mL, 20.0 mmol) was added and the reaction stirred at room temperature for 40 minutes after which time the suspension dissolved. The reaction mixture was quenched with sodium thiosulphate solution (15 mL) and stirred for 5 minutes. The mixture was extracted with ethyl acetate (2×30 mL) and the combined organic extracts washed with brine (30 mL), dried over magnesium sulfate and filtered. The solvent was removed under reduced pressure to give a yellow oil (3.66 g). Purification using silica gel column chromatography eluting with a gradient of dichloromethane/methanol (100:0 to 80:20) to afford the title compound (1.94 g, 73%) as a white solid.

¹H NMR (400 MHz, CDCl₃): δ ppm 1.02 (d, 6H), 2.11 (m, 1H), 4.05 (d, 2H), 6.03 (br s, 1H), 7.31 (d, 1H), 7.65 (d, 1H)

LCMS Rt=2.51 minutes MS m/z 200 [M−H]⁻

### Preparation 6

### 4-(4-chloro-2-methoxyphenoxy)benzoic acid

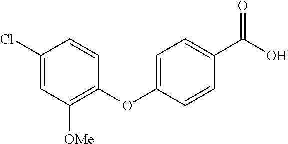 [SMILES: COc1cc(Cl)ccc1Oc1ccc(C(=O)O)cc1]

Prepared according to Method F (Preparation 2) using 4-(4-chloro-2-methoxyphenoxy)benzonitrile (Preparation 7) for 48 hours to afford the title compound as a white solid.

¹H NMR (400 MHz, CDCl₃): δ ppm 3.73 (s, 3H), 6.87 (d, 2H), 7.05 (dd, 1H), 7.17 (d, 1H), 7.28 (d, 1H), 7.87 (d, 2H).

LCMS Rt=1.60 minutes MS m/z 279 [MH]⁺

### Preparation 7

### 4-(4-chloro-2-methoxyphenoxy)benzonitrile

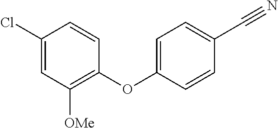 [SMILES: COc1cc(Cl)ccc1Oc1ccc(C#N)cc1]

Prepared according to Method E (Preparation 1) using 4-chloro-2-methoxyphenol and 4-fluorobenzonitrile at 100° C. for 18 hours to afford the title compound as a white solid.

¹H NMR (400 MHz, CDCl₃): δ ppm 3.77 (s, 3H), 6.88-6.94 (m, 2H), 6.95-6.99 (m, 1H), 6.99-7.03 (m, 2H), 7.54-7.60 (m, 2H).

LCMS Rt=1.75 minutes MS m/z 260 [MH]⁺

### Preparation 8

### 4-(4-chloro-2-pyridazin-4-ylphenoxy)benzoic acid hydrochloride salt

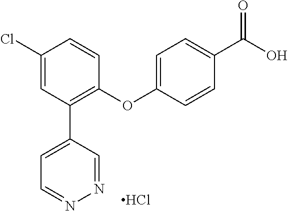 [SMILES: O=C(O)c1ccc(Oc2ccc(Cl)cc2-c2ccnnc2)cc1 ; Cl]

Prepared according to Method F (Preparation 2) using 4-(4-chloro-2-pyridazin-4-ylphenoxy)benzonitrile (Preparation 9) to afford the title compound as the hydrochloride salt.

¹H NMR (400 MHz, CDCl₃): δ ppm 7.02 (d, 2H), 7.22 (d, 1H), 7.62 (dd, 1H), 7.82-7.91 (m, 4H), 9.24 (dd, 1H), 9.39 (dd, 1H).

LCMS Rt=1.43 minutes MS m/z 327 [MH]⁺

### Preparation 9

### 4-(4-chloro-2-pyridazin-4-ylphenoxy)benzonitrile

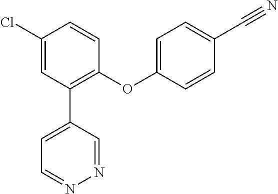 [SMILES: N#Cc1ccc(Oc2ccc(Cl)cc2-c2ccnnc2)cc1]

Prepared according to Method E (Preparation 1) using 4-chloro-2-pyridazin-4-ylphenol (Preparation 24) and 4-fluorobenzonitrile at 120° C. for 18 hours to afford the title compound as a white solid.

¹H NMR (400 MHz, CDCl₃): δ ppm 6.91 (d, 2H), 7.09 (d, 1H), 7.42-7.42 (m, 5H), 9.19-9.21 (m, 1H), 9.37-9.39 (m, 1H).

LCMS Rt=1.19 minutes MS m/z 308 [MH]⁺

### Preparation 10

### 4-(4-chloro-2-methoxyphenoxy)-2,5-difluorobenzoic acid

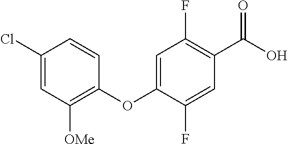 [SMILES: COc1cc(Cl)ccc1Oc1cc(F)c(C(=O)O)cc1F]

Prepared according to Method G (Preparation 4) using methyl 4-(4-chloro-2-methoxyphenoxy)-2,5-difluorobenzoate (Preparation 11) to afford the title compound as a white solid.

¹H NMR (400 MHz, CDCl₃): δ ppm 3.77 (s, 3H), 6.64 (dd, 1H), 7.06 (dd, 1H), 7.24 (d, 1H), 7.31 (d, 1H), 7.76 (dd, 1H), 13.31-13.37 (br.s, 1H).

LCMS Rt=1.48 minutes MS m/z 315 [MH]⁺

### Preparation 11

### Methyl 4-(4-chloro-2-methoxyphenoxy)-2,5-difluorobenzoate

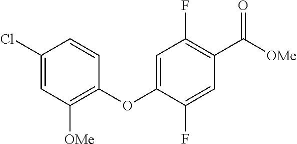 [SMILES: COC(=O)c1cc(F)c(Oc2ccc(Cl)cc2OC)cc1F]

To a solution of 4-chloro-2-methoxyphenol (391 mg, 2.5 mmol) in DMSO (10 mL) was added potassium carbonate (682 mg, 4.9 mmol) followed by methyl 2,4,5-trifluorobenzoate (469 mg, 2.5 mmol). The resulting mixture was heated to 50° C. with stirring for 5 hours. The mixture was then cooled and diluted with water (30 mL) then washed with EtOAc (3×30 mL). The combined organic layers were dried over magnesium sulfate, filtered and concentrated in vacuo. Purification by silica gel column chromatography eluting with a gradient of EtOAc/heptane (0:100 to 25:75) afforded the title compound (768 mg, 95%) as a white solid.

¹H NMR (400 MHz, CDCl₃): δ ppm 3.76 (s, 3H), 3.81 (s, 3H), 6.68 (dd, 1H), 7.07 (dd, 1H), 7.26 (d, 1H), 7.31 (d, 1H), 7.80 (dd, 1H).

LCMS Rt=1.80 minutes MS m/z 329 [MH]⁺

### Preparation 12

### 4-(2-methoxyphenoxy)benzonitrile

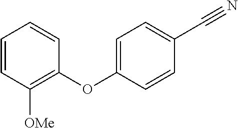 [SMILES: COc1ccccc1Oc1ccc(C#N)cc1]

To a solution of 2-methoxyphenol (100 mg, 0.81 mmol) in DMSO (5 mL) was added potassium carbonate (223 mg, 1.6 mmol) followed by 4-fluorobenzonitrile (98 mg, 0.81 mmol). The resulting mixture was heated to 120° C. with stirring for 18 hours. The mixture was cooled, diluted with water (30 mL), then washed with EtOAc (3×30 mL). The combined organic layers were dried over magnesium sulfate, filtered and concentrated in vacuo to afford the title compound (161 mg, 89%).

¹H NMR (400 MHz, CDCl₃): δ ppm 3.79 (s, 3H), 6.92 (d, 2H), 6.97-7.10 (m, 3H), 7.22 (d, 1H), 7.56 (d, 2H).

LCMS Rt=1.34 minutes MS m/z 226 [MH]⁺

### Preparation 13

### 4-(3-methoxyphenoxy)benzonitrile

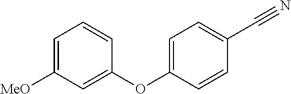 [SMILES: COc1cccc(Oc2ccc(C#N)cc2)c1]

To a solution of 3-methoxyphenol (100 mg, 0.81 mmol) in DMSO (5 mL) was added potassium carbonate (223 mg, 1.6 mmol) followed by 4-fluorobenzonitrile (98 mg, 0.81 mmol). The resulting mixture was heated to 120° C. with stirring for 18 hours. The mixture was cooled, diluted with water (30 mL), then washed with EtOAc (3×30 mL). The combined organic layers were dried over magnesium sulfate, filtered and concentrated in vacuo to afford the title compound (177 mg, 98%).

¹H NMR (400 MHz, CDCl₃): δ ppm 3.80 (s, 3H), 6.59-6.67 (m, 2H), 6.99-7.06 (m, 2H), 7.30 (dd, 1H), 7.56-7.63 (m, 3H).

LCMS Rt=1.40 minutes MS m/z 226 [MH]⁺

### Preparation 14

### 4-[(5-chloro-6-isobutoxypyridin-3-yl)oxy]benzonitrile

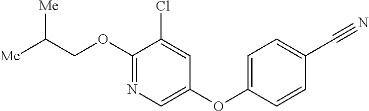 [SMILES: CC(C)COc1ncc(Oc2ccc(C#N)cc2)cc1Cl]

To a solution of 5-chloro-6-isobutoxypyridin-3-ol (Preparation 5, 89 mg, 0.44 mmol) in DMSO (5 mL) was added potassium carbonate (122 mg, 0.88 mmol) followed by 4-fluorobenzonitrile (53 mg, 0.44 mmol). The resulting mixture was heated to 110° C. with stirring for 18 hours. The mixture was cooled, diluted with water (25 mL), then washed with EtOAc (3×25 mL). The combined organic layers were dried over magnesium sulfate, filtered and concentrated in vacuo to afford the title compound as a yellow gum (118 mg, 88%).

¹H NMR (400 MHz, CDCl₃): δ ppm 1.05 (d, 6H), 2.09-2.22 (m, 1H), 4.14 (d, 2H), 6.99 (d, 2H), 7.44 (d, 1H), 7.62 (d, 2H), 7.88 (d, 1H)

LCMS Rt=1.94 minutes MS m/z 303 [MH]⁺

### Preparation 15

### Methyl 2,5-difluoro-4-(2-methoxyphenoxy)benzoate

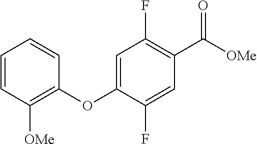 [SMILES: COC(=O)c1cc(F)c(Oc2ccccc2OC)cc1F]

To a solution of 2-methoxyphenol (116 mg, 0.79 mmol) in DMSO (3 mL) was added potassium carbonate (218 mg, 1.6 mmol) followed by methyl 2,4,5-trifluorobenzoate (150 mg, 0.79 mmol). The resulting mixture was heated to 50° C. with stirring for 18 hours. The mixture was then cooled and diluted with water (30 mL) then washed with EtOAc (3×30 mL). The combined organic layers were dried over magnesium sulfate, filtered and concentrated in vacuo to afford the title compound (121 mg, 52%) as a yellow gum.

¹H NMR (400 MHz, CDCl₃): δ ppm 3.81 (s, 3H), 3.91 (s, 3H), 6.41 (dd, 1H), 6.98-7.03 (m, 2H), 7.05 (dd, 1H), 7.24-7.29 (m, 1H), 7.75 (dd, 1H).

LCMS Rt=1.72 minutes MS m/z 295 [MH]⁺

### Preparation 16

### Methyl 2,5-difluoro-4-(3-methoxyphenoxy)benzoate

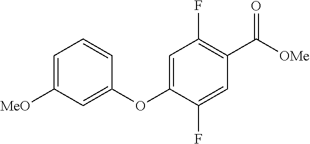 [SMILES: COC(=O)c1cc(F)c(Oc2cccc(OC)c2)cc1F]

To a solution of 3-methoxyphenol (116 mg, 0.79 mmol) in DMSO (3 mL) was added potassium carbonate (218 mg, 1.6 mmol) followed by methyl 2,4,5-trifluorobenzoate (150 mg, 0.79 mmol). The resulting mixture was heated to 50° C. with stirring for 18 hours. The mixture was then cooled and diluted with water (30 mL) then washed with EtOAc (3×30 mL). The combined organic layers were dried over magnesium sulfate, filtered and concentrated in vacuo to afford the title compound (154 mg, 66%) as an orange gum.

¹H NMR (400 MHz, CDCl₃): δ ppm 3.81 (s, 3H), 3.92 (s, 3H), 6.61-6.69 (m, 3H), 6.76-6.81 (m, 1H), 7.31 (t, 1H), 7.76 (dd, 1H).

LCMS Rt=1.76 minutes MS m/z 295 [MH]⁺

### Preparation 17

### 4-methylphenyl 4-[(5-chloro-6-isopropoxypyridin-3-yl)oxy]-2,5-difluorobenzoate-d7

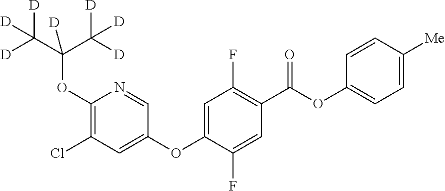 [SMILES: [2H]C([2H])([2H])C([2H])(Oc1ncc(Oc2cc(F)c(C(=O)Oc3ccc(C)cc3)cc2F)cc1Cl)C([2H])([2H])[2H]]

To a solution of 5-chloro-6-isopropoxypyridin-3-ol-d7 (Preparation 21, 300 mg, 1.5 mmol) in DMSO (15 mL) was added potassium carbonate (739 mg, 5.3 mmol) followed by 4-Methylphenyl 2,4,5-trifluorobenzoate (Preparation 45, 419 mg, 1.6 mmol). The resulting mixture was stirred at room temperature for 18 hours then diluted with water (50 mL) then extracted with EtOAc (3×50 mL). The combined organic layers were dried over magnesium sulfate, filtered and concentrated in vacuo to afford the title compound (499 mg, 73%) as a pale yellow solid.

¹H NMR (400 MHz, CDCl₃): δ ppm 2.38 (s, 3H), 6.66-6.71 (m, 1H), 7.08-7.10 (m, 2H), 7.22-7.24 (m, 2H), 7.48 (d, 1H), 7.91-7.95 (m, 2H).

LCMS Rt=1.86 minutes MS m/z 442 [MH]⁺

### Preparation 18

### 4-(biphenyl-2-yloxy)-3-cyanobenzoic acid

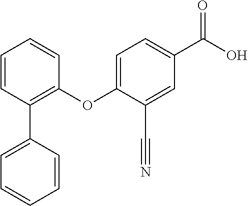 [SMILES: N#Cc1cc(C(=O)O)ccc1Oc1ccccc1-c1ccccc1]

To a suspension of 3-cyano-4-fluorobenzoic acid (500 mg, 3.0 mmol) in DMSO (10 mL) were added biphenyl-2-ol (773 mg, 4.5 mmol) and potassium carbonate (1.3 g, 0.1 mmol). The resulting mixture was stirred at 80° C. for 48 hours, then cooled and diluted with 2N HCl (100 mL) and stirred for a further 2 hours at room temperature. The mixture was extracted with ethyl acetate (2×100 mL) and the combined organic extracts dried over magnesium sulfate and filtered and concentrated in vacuo. Purification by silica gel column chromatography eluting with a gradient of EtOAc:heptane (0:100 to 50:50) afforded the title compound (672 mg, 70%) as an orange solid.

¹H NMR (400 MHz, CDCl₃): δ ppm 6.75 (d, 1H) 7.12-7.64 (m, 9H) 8.00 (dd, 1H) 8.19 (d, 1H) 13.26 (br s, 1H).

MS m/z 316 [MH]⁺

### Preparation 19

### 4-[(5-chloro-6-fluoropyridin-3-yl)oxy]-2,5-difluorobenzoic acid

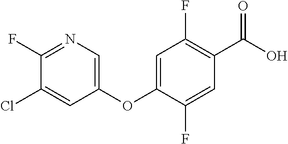 [SMILES: O=C(O)c1cc(F)c(Oc2cnc(F)c(Cl)c2)cc1F]

To a solution of tert-butyl 4-[(5-chloro-6-fluoropyridin-3-yl)oxy]-2,5-difluorobenzoate (Preparation 20, 10.0 g, 27.85 mmol) in dichloromethane (100 mL) was added trifluoroacetic acid (120 mL) dropwise at room temperature. The reaction mixture was stirred at room temperature under nitrogen for 4 hours. The mixture was concentrated in vacuo and the resulting residue purified by A-HPLC preparation to give the title compound (7.5 g, 89%) as a white solid.

¹H NMR (400 MHz, d₆-DMSO): δ ppm 7.23-7.28 (m, 1H), 7.79-7.84 (m, 1H), 8.19-8.20 (m, 1H), 8.28-8.31 (m, 1H), 13.36 (s, 1H).

LCMS Rt=2.64 minutes MS m/z 304 [MH]⁺

### Preparation 20

### tert-butyl 4-[(5-chloro-6-fluoropyridin-3-yl)oxy]-2,5-difluorobenzoate

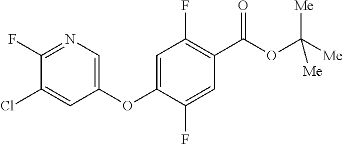 [SMILES: CC(C)(C)OC(=O)c1cc(F)c(Oc2cnc(F)c(Cl)c2)cc1F]

To a solution of tert-butyl 2,4,5-trifluorobenzoate (15.8 g, 68 mmol) in DMSO (150 mL) was added potassium carbonate (28 g, 204 mmol), followed by addition of 5-chloro-6-fluoropyridin-3-ol (10 g, 68 mmol) in one portion. The mixture was stirred at room temperature for 18 hours. Water (200 mL) was added and the mixture was extracted with ethyl acetate (3×100 mL). The combined organic phases were dried over anhydrous Na₂SO₄, filtered and concentrated in vacuo. The crude product was purified using flash column chromatography eluting with petroleum ether/ethyl acetate (100:1 to 5:1) to give the title compound (13 g, 54%) as a white solid.

¹H NMR (400 MHz, MeOD): δ ppm 1.6 (s, 9H), 7.01-7.05 (m, 1H), 7.72-7.76 (m, 1H), 7.91-7.94 (m, 1H), 7.99-8.01 (m, 1H).

LCMS Rt 4.27 min MS m/z no mass ion observed

### Preparation 21

### 5-chloro-6-isopropoxypyridin-3-ol-d7

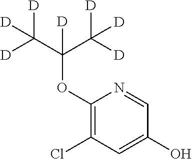 [SMILES: [2H]C([2H])([2H])C([2H])(Oc1ncc(O)cc1Cl)C([2H])([2H])[2H]]

An solution of potassium peroxymonosulfate (24.9 g, 38.5 mmol) in water (100mL) was added dropwise to a solution of 3-chloro-2-disopropoxy-5-(4,4,5,5-tetramethyl-1,3,2-dioxaborolan-2-yl)pyridine-d7 (Preparation 22, 9.8 g, 32.1 mmol) in acetone (100 mL) under a nitrogen atmosphere at 0° C. The mixture was stirred at 0° C. for 1 hours, diluted with water (150 mL) and extracted with tertbutylmethylether (2×200 mL). The combined organic layers were washed with sodium metabisulfite (100 mL), brine (100 mL), then dried over sodium sulfate, filtered and concentrated in vacuo to yield pale yellow oil (7.57 g). The resulting oil was purified by silica gel column chromatography eluting with 100% heptane to 8:2 heptane/ethyl acetate to yield the title compound as a white solid (4.26 g, 68%).

¹H NMR (400 MHz, CDCl₃): δ ppm 4.77 (s, 1H), 7.28 (d, 1H), 7.68-7.69 (d, 1H).

LCMS Rt=1.19 minutes MS m/z 195 [MH]⁺

### Preparation 22

### 3-chloro-2-isopropoxy-5-(4,4,5,5-tetramethyl-1,3,2-dioxaborolan-2-yl)pyridine-d7

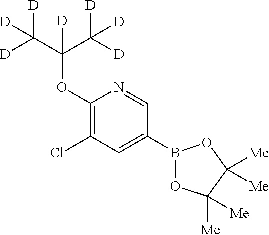 [SMILES: [2H]C([2H])([2H])C([2H])(Oc1ncc(B2OC(C)(C)C(C)(C)O2)cc1Cl)C([2H])([2H])[2H]]

To a solution of 3-chloro-2-isopropoxypyridine-d7 (Preparation 23, 5.37 g, 30.1 mmol) in heptane (55 mL) was added bis(pinacolato)diboron (9.16 g, 36.1 mmol) and 4,4-di-tert-butyl-2,2-dipyridyl (81 mg, 0.30 mmol). The mixture was degassed then flushed with nitrogen, before the addition of di-μ-methanolatodiiridium(Ir-Ir)-cycloocta-1,5-diene (1:2) (204 mg, 0.30 mmol). The reaction was stirred at room temperature for 18 hours. The resulting mixture was purified using silica gel column chromatography eluting with heptane (100%) to heptane/ethylacetate (7:3) to afford the title compound as yellow oil (9.8 g, 107%). The product was carried through to the next step without further purification.

¹H NMR (400 MHz, CDCl₃): δ ppm 1.34 (s, 12H), 7.95-7.96 (d, 1H), 8.38 (d, 1H).

LCMS Rt=1.81 minutes MS m/z 305 [MH]⁺

### Preparation 23

### 3-chloro-2-d7-isopropoxypyridine

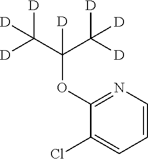 [SMILES: [2H]C([2H])([2H])C([2H])(Oc1ncccc1Cl)C([2H])([2H])[2H]]

A solution of d8-isopropyl alcohol (4.71 mL, 61.5 mmol) in anhydrous THF (10 mL) was added slowly over 1 min to a suspension of NaH (60% in mineral oil) (2.46 g, 61.5 mmol) in anhydrous THF (50 mL). After 10 minutes a solution of 2-fluoro-3-chloropyridine (5.05 g, 38.4 mmol) in THF (10 mL) was added over 5 minutes at 5° C. (ice bath). The reaction was then warmed to room temperature and stirred for 18 hours. The reaction was diluted with THF (20 mL), cooled to 5° C. (ice bath) and quenched with water (50 mL). The mixture was extracted with EtOAc (50 mL) and brine. The organic layer was dried over magnesium sulfate, filtered, and concentrated in vacuo to afford a crude oil which was purified by silica gel column chromatography eluting with 0 to 30% EtOAc in heptane to yield the title compound as a colourless oil (5.37 g, 53%).

¹H NMR (400 MHz, CDCl₃): δ ppm 6.78-6.81 (m, 1H), 7.60-7.62 (m, 1H), 8.03-8.04 (m, 1H).

LCMS Rt=1.41 minutes MS m/z no mass ion observed

### Preparation 24

### 2-pyridazin-4-yl-4-chlorophenol

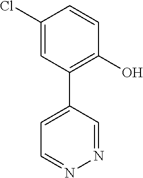 [SMILES: Oc1ccc(Cl)cc1-c1ccnnc1]

To a degassed solution of (5-chloro-2-hydroxyphenyl)boronic acid (254 mg, 1.47 mmol) in toluene (4 mL) and EtOH (0.5 mL), was added 4-bromopyridazine hydrobromide (WO2010079443, 354 mg, 1.47 mmol) and tetrakis(triphenylphospine) palladium (86 mg, 0.074 mmol). A degassed 2M aqueous solution of Na₂CO₃ (624 mg, 5.89 mmol, 2.94 mL) was then added. The reaction mixture was heated to 110° C. under nitrogen for 3 hours. The reaction was cooled, filtered through celite, washing with EtOAc. The filtrate was concentrated in vacuo to a afford a solid, which was slurried in EtOAc, filtered, washed with EtOAc and dried to afford the title compound as a white solid (225 mg, 74%).

¹H NMR (500MHz, MeOD): δ ppm 6.97 (d, 1H), 7.35-7.28 (m, 1H), 7.52 (d, 1H), 7.98 (dd, 1H), 9.18 (dd, 1H), 9.52-9.47 (m, 1H).

### Preparation 25

### 4-((5-chloro-6-isopropoxypyridin-3-yl)oxy)-2,5-difluorobenzamide

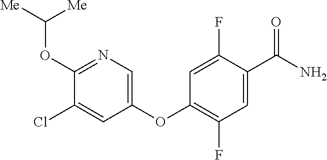 [SMILES: CC(C)Oc1ncc(Oc2cc(F)c(C(N)=O)cc2F)cc1Cl]

A solution of 5-chloro-6-isopropoxypyridin-3-ol (Preparation 30, 490 mg, 2.61 mmol), trifluorobenzonitrile (411 mg, 2.61 mmol), and K₂OC₃ (1085 mg, 7.86 mmol) in DMSO (10 mL) was stirred at room temperature for 2.5 hours. The solution was then cooled to 0° C., further K₂OC₃ (1500 mg, 10.3 mmol) was added followed by H₂O₂ (30%, 5.0 mL, 44 mmol) and stirred for 18 hours at room temperature under nitrogen. The mixture was diluted with H₂O (80 mL) and stirred for 2 hours, followed by extraction with EtOAc (3×30 mL). The combined organic layers were washed with H₂O (10 mL), and then brine (30 mL), dried over MgSO₄, filtered and the solvent removed in vacuo to give the title compound (960 mg, 107%) as a colourless solid.

¹H NMR (400 MHz, d₆-DMSO): δ ppm 1.30 (m, 6H), 5.25 (m, 1H), 7.10 (m, 1H), 7.65 (m, 3H), 7.90 (m, 1H), 8.07 (m, 1H).

LCMS Rt=3.27 minutes MS m/z 343 [MH]⁺

### Preparation 26

### 4-((5-chloro-6-isopropoxypyridin-3-yl)oxy)-2,5-difluoro-N-(N-(4-methoxybenzyl)N-methylsulfamoyl)benzamide

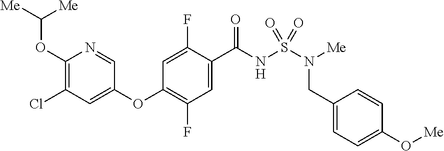 [SMILES: COc1ccc(CN(C)S(=O)(=O)NC(=O)c2cc(F)c(Oc3cnc(OC(C)C)c(Cl)c3)cc2F)cc1]

A solution of 4-((5-chloro-6-isopropoxypyridin-3-yl)oxy)-2,5-difluorobenzoic acid (Preparation 27, 92 mg, 0.270 mmol), N-(4-methoxybenzyl)-N-methylsulfamide (124 mg, 0.539 mmol), EDCl (128 mg, 0.671 mmol), DMAP (82 mg, 0.671 mmol), and diisopropylethylamine (0.12 mL, 0.671 mmol) in DCM (10 mL) was stirred at room temperature for 96 hours under an inert atmosphere. The mixture was diluted with 2M HCl (10 mL), and extracted with DCM (3×10 mL). The combined organic layers were dried over MgSO₄ and concentrated in vacuo and the crude material purified by reverse phase chromatography eluting with 100:0:0.1 to 0:100:0.1 (H₂O:MeCN:HCO₂H) to give the title compound (16 mg, 11%) as a colourless solid.

¹H NMR (400 MHz, CDCl₃): δ ppm 1.40 (d, 6H), 1.90 (s, 3H), 3.80 (s, 3H), 4.45 (s, 2H), 5.30 (s, 1H), 5.32 (m, 1H), 6.62 (m, 1H), 6.85 (m, 2H), 7.25 (m, 2H), 7.48 (m, 1H), 7.90 (m, 2H).

LCMS Rt=3.91 minutes MS m/z 554 [M−H]⁻

### Preparation 27

### 4-((5-chloro-6-isopropoxypyridin-3-yl)oxy)-2,5-difluorobenzoic acid

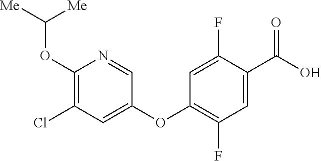 [SMILES: CC(C)Oc1ncc(Oc2cc(F)c(C(=O)O)cc2F)cc1Cl]

A solution of 5-chloro-6-isopropoxypyridin-3-ol (Preparation 30, 350 mg, 1.87 mmol), 2,4,5-trifluorobenzoic acid (330 mg, 1.87 mmol) and K₂OC₃ (1033 mg, 7.49 mmol) in DMSO (5 mL) was stirred for 18 hours at 170° C. under an inert atmosphere. The reaction was cooled and diluted with 2M HCl (50 mL). The mixture was extracted with EtOAc (3×20 mL), the combined organic layers dried over MgSO₄, filtered and the solvent removed in vacuo. The crude material was purified by reverse phase chromatography eluting with 100:0:0.1 to 0:100:0.1 (H₂O:MeCN:HCO₂H) to give the title compound as an orange solid (353 mg, 55%).

LCMS Rt=3.20 minutes MS m/z 342 [M−H]⁻

### Preparation 28

### N-[(dimethylamino)sulfonyl]-2,4,5-trifluorobenzamide

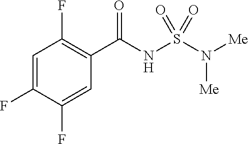 [SMILES: CN(C)S(=O)(=O)NC(=O)c1cc(F)c(F)cc1F]

A solution of 2,4,5-trifluorobenzoic acid (993 mg, 5.64 mmol), N-(3-Dimethylaminopropyl)-N′-ethylcarbodiimide hydrochloride (1.62 g, 8.45 mmol), 4-(Dimethylamino)pyridine (69.0 mg, 0.565 mmol) and triethylamine (1.00 mL, 7.17 mmol) in dichloromethane (20 mL) was stirred at room temperature under nitrogen for 15 minutes. N,N-dimethylsulfamide (Preparation 29, 1.05 g, 8.46 mmol) and triethylamine (1.36 mL, 9.76 mmol) in dichloromethane (20 mL) was added and the reaction was stirred for 4 days at room temperature under nitrogen. The reaction was concentrated in vacuo and partitioned between ethyl acetate (20 mL) and 2M aqueous hydrochloric acid (20 mL). The aqueous layer was extracted with ethyl acetate (2×20 mL). The combined organic layers were washed with brine (30 mL), dried over magnesium sulphate, filtered and concentrated in vacuo to afford a brown oil. Azeotroping the oil with ethyl acetate and heptane afforded a solid which was recrystallised from ethyl acetate and heptanes to afford the title compound as a white solid (228 mg, 14%)

¹H NMR (400 MHz, CDCl₃): δ ppm 3.05 (s, 6H), 7.10 (dd, 1H), 7.95 (dd, 1H), 8.70 (br s, 1H).

LCMS Rt=2.58 minutes MS m/z no mass ion observed

### Preparation 29

### N,N-dimethylsulfamide

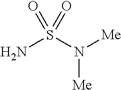

N,N-Dimethylsulfamoyl chloride (1.00 mL, 9.31 mmol) was added to a 30% aqueous ammonia solution (5 mL) at 0° C. The reaction was stirred for 3 hours and solvent removed in vacuo to afford a white solid. The solid was sonicated with acetone (20 mL), filtered and the solid was washed with additional acetone (20 mL). The combined organic filtrate solvent was concentrated in vacuo to afford the title compound as a white solid (1082 mg, 94%).

¹H NMR (400 MHz, acetone-d6): δ ppm 2.70 (s, 6H), 5.90 (br s, 2H).

### Preparation 30

### 5-Chloro-6-isopropoxypyridin-3-ol

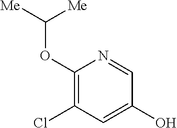 [SMILES: CC(C)Oc1ncc(O)cc1Cl]

Method H

Peracetic acid (191 mL, 1077 mmol) was added to a solution of 3-chloro-2-isopropoxy-5-(4,4,5,5-tetramethyl-1,3,2-dioxaborolan-2-yl)pyridine (Preparation 31, 479 g of crude material, 898 mmol if 100% yield in previous step) in aqueous acetic acid at 5-10° C. The reaction was warmed slowly to room temperature over 4 hours, concentrated to 10% volume and extracted with EtOAc. The resulting crude material was purified by silica gel column chromatography eluting with 50% EtOAc in heptane to afford the title compound as a white solid (110 g, 65% over two steps).

¹H NMR (400 MHz, CDCl₃): δ ppm 1.35 (d, 6H), 5.19 (m, 1H), 7.26 (d, 1H), 7.68 (d, 1H).

LCMS Rt=2.10 minutes MS m/z 186 [M−H]⁻

### Preparation 31

### 3-Chloro-2-isopropoxy-5-(4,4,5,5-tetramethyl-1,3,2-dioxaborolan-2-yl)pyridine

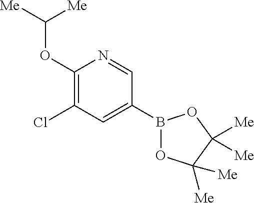 [SMILES: CC(C)Oc1ncc(B2OC(C)(C)C(C)(C)O2)cc1Cl]

Method I

3-Chloro-2-isopropoxypyridine (Preparation 32, 154.1 g, 897.9 mmol), bis(pinacolato)diboron (273.6 g, 1077.4 mmol), 4,4-di-tert-butyl-2,2-dipyridyl (2.459 g, 8.979 mmol) were added to heptane (400 mL), followed by di-μ-methanolatodiiridium(Ir-Ir)-cycloocta-1,5-diene (1:2) (0.193 g, 0.2914 mmol). The reaction was stirred at room temperature for 18 hours, quenched with MeOH and concentrated to dryness to afford the title compound as red-brown oil (479 g). The product was carried through to the next step without further purification.

¹H NMR (400 MHz, CDCl₃): δ ppm 1.32 (s, 12H), 1.38 (d, 6H), 5.41 (m, 1H), 7.94 (d, 1H), 8.37 (d, 1H).

LCMS Rt=5.10 minutes m/z 296 [M−H]⁻

### Preparation 32

### 3-Chloro-2-isopropoxypyridine

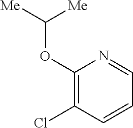 [SMILES: CC(C)Oc1ncccc1Cl]

iso-Propanol (128 mL; 1.07 mol) was added dropwise over 50 minutes to a suspension of sodium hydride (64.10 g; 1.07 mol) in THF (1.65 L) at 5° C. The reaction mixture was then warmed to room temperature and stirred for 1 hour, then 2,3-dichloropyridine (154.6 g; 1.11 mol) was added and the reaction mixture was heated to a gentle reflux for 18 hours. The reaction mixture was cooled to 5-10° C. and carefully quenched with brine:water (50:50; 100 mL), followed by water (300 mL). The aqueous layer was extracted with EtOAc (3×600 mL). The combined organic layers were washed with brine, dried over anhydrous magnesium sulfate, filtered and evaporated to afford the title compound as a dark red oil (164 g, 89%).

¹H NMR (400 MHz, CDCl₃): δ ppm 1.40 (d, 6H), 5.36 (m, 1H), 6.80 (m, 1H), 7.6 (m, 1H), 8.05 (m, 1H).

LCMS Rt=3.09 minutes MS m/z no mass ion observed

The following Preparations were prepared by Method H as described for Preparation 30, using appropriate reagents and conditions.

| Prep | Name | Data |
|---|---|---|
| 33 | 5-chloro-6-(2,2,2- | LCMS Rt = 2.61 minutes |
| | trifluoroethoxy) | MS m/z 228[MH]⁺ |
| | pyridin-3-ol | Prepared using Preparation 38. |
| 34 | 5-chloro-6- | ¹H NMR (400 MHz, CDCl₃): |
| | (cyclopropylmethoxy) | δ ppm 0.30 (m, 2H), 0.55 (m, 2H), |
| | pyridin-3-ol | 1.25 (m, 1H), 4.10 (s, 2H), 4.85 |
| | | (br, 1H), 7.30 (s, 1H), 7.60 (s, 1H). |
| | | Prepared using Preparation 36. |

### Preparation 35

### 5-chloro-6-(2-fluoro-2-methylpropoxy)pyridin-3-ol

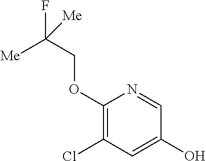 [SMILES: CC(C)(F)COc1ncc(O)cc1Cl]

Hydrogen peroxide solution (30%, 0.462 mL, 4.52 mmol) was added in five portions to a solution of 3-chloro-2-(2-fluoro-2-methylpropoxy)-5-(4,4,5,5-tetramethyl-1,3,2-dioxaborolan-2-yl)pyridine (Preparation 37, 1.28 g, 3.77 mmol) in MeOH/H₂O (30 mL: 10 mL) at 0° C. The reaction mixture was stirred at room temperature for 3 hours. An aqueous solution of sodium thiosulfate (0.1M, 20 mL) was added and the mixture stirred at room temperature for 5 minutes then extracted with EtOAc (50 mL). The organic extracts were washed with brine (2×30 mL), dried over magnesium sulfate and concentrated in vacuo to afford the crude material as a yellow oil. The crude material was purified by silica gel column chromatography eluting with 0% to 60% EtOAc in heptane to afford the title compound as a white solid.

¹H NMR (400 MHz, CDCl₃): δ ppm 1.45 (s, 3H), 1.50 (s, 3H), 4.30 (d, 2H), 4.95 (m, 1H), 7.30 (s, 1H), 7.65 (s, 1H).

The following Preparations were prepared by Method I described for Preparation 31, using appropriate reagents and conditions.

| Prep | Name | Data |
|---|---|---|
| 36 | 3-chloro-2- | ¹H NMR (400 MHz, CDCl₃): |
| | (cyclopropylmethoxy)- | δ ppm 0.41-0.38 (m, 2H), |
| | 5-(4,4,5,5-tetramethyl- | 0.64-0.59 (m, 2H), 1.38-1.24 (m, 13H), |
| | 1,3,2-dioxaborolan-2- | 4.27 (d, 2H), 7.97 (d, 1H), 8.37 (d, 1H). |
| | yl)pyridine | Prepared using Preparation 39. |
| 37 | 3-chloro-2-(2-fluoro-2- | ¹H NMR (400 MHz, CDCl₃): |
| | methylpropoxy)-5- | δ ppm 1.35 (s, 12H), 1.50 (s, 3H), |
| | (4,4,5,5-tetramethyl- | 1.55 (s, 3H), 4.40 (d, 2H), |
| | 1,3,2-dioxaborolan-2- | 8.00 (s, 1H), 8.40 (s, 1H). |
| | yl)pyridine | Prepared using Preparation 40. |
| 38 | 3-chloro-5-(4,4,5,5- | ¹H NMR (400 MHz, CDCl₃): |
| | tetramethyl-1,3,2- | δ ppm 1.35 (s, 12H), 4.86 (m, 2H), |
| | dioxaborolan-2-yl)-2- | 8.05 (d, 1H), 8.38 (d, 1H). |
| | (2,2,2-trifluoroethoxy) | Prepared using Preparation 41. |
| | pyridine | |

The following Preparations were prepared by Method J described for Preparation 32 above, using appropriate reagents and conditions.

| Prep | Name | Data |
|---|---|---|
| 39 | 3-chloro-2- | ¹H NMR (400 MHz, CDCl₃): |
| | (cyclopropylmethoxy) | δ ppm 0.03-0.01 (m, 2H), |
| | pyridine | 0.26-0.21 (m, 2H), 0.96 (m, 1H), |
| | | 3.85 (d, 2H), 6.44 (m, 1H), |
| | | 7.24 (m, 1H), 7.64 (m, 1H). |
| 40 | 3-chloro-2-(2-fluoro- | ¹H NMR (400 MHz, CDCl₃): |
| | 2-methylpropoxy) | δ ppm 1.50 (s, 3H), 1.55 (s, 3H), |
| | pyridine | 4.40 (d, 2H), 6.85 (m, 1H), |
| | | 7.65 (d, 1H), 8.00 (d, 1H). |
| 41 | 3-chloro-2-(2,2,2- | ¹H NMR (400 MHz, CDCl₃): |
| | trifluoroethoxy) | δ ppm 4.83 (m, 2H), 6.96 (m, 1H), |
| | pyridine | 7.70 (m, 1H), 8.05 (m, 1H). |

### Preparation 42

### N-[(dimethylamino)sulfonyl]-2,5-difluoro-4-hydroxybenzamide

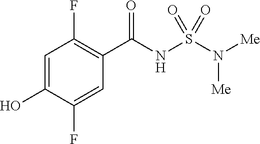 [SMILES: CN(C)S(=O)(=O)NC(=O)c1cc(F)c(O)cc1F]

Potassium tert-butoxide (568 mg, 5.1 mmol) was added to a solution of N-[(dimethylamino)sulfonyl]-2,4,5-trifluorobenzamide (Preparation 28, 650 mg, 2.3 mmol) in DMSO (5 mL). The reaction mixture was stirred at room temperature for 18 hours then at 100° C. for 2 hours. The reaction mixture was quenched with 10% aqueous solution of citric acid (10 mL) and extracted with ethyl acetate (3×10 mL). The combined organic layers were washed with brine (10 mL), dried over MgSO₄, filtered and concentrated in vacuo. The crude compound was purified using silica gel column chromatography eluting with heptanes:ethyl acetate (from 90:10 to 20:80) to give the title compound as a colourless solid (237 mg, 37%).

¹H NMR (400 MHz, MeOD): δ ppm 3.95 (s, 6H), 6.65-6.78 (m, 1H), 7.38-7.47 (m, 1H).

LCMS Rt=2.19 minutes MS m/z 279 [M−H]⁻

### Preparation 43

### tert-butyl 4-((5-chloro-6-isopropoxypyridin-3-yl)oxy)-2,5-difluorobenzoate

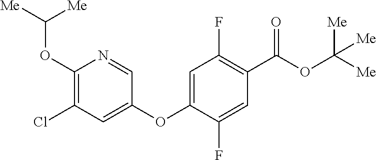 [SMILES: CC(C)Oc1ncc(Oc2cc(F)c(C(=O)OC(C)(C)C)cc2F)cc1Cl]

To a solution of tert-butyl 2,4,5-trifluorobenzoate (J.O.C. 68 (3), 770-778, (2003), 582 mg, 2.51 mmol) in dimethylsulfoxide (13 mL) was added 5-chloro-6-isopropoxypyridin-3-01 (Preparation 30, 470 mg, 2.51 mmol) followed by potassium carbonate (691 mg, 5.1 mmol). The reaction was stirred for 18 hours at room temperature and then water (10 mL) was added. The reaction mixture was extracted into ethyl acetate (3×20 mL). The combined organic layers were dried over magnesium sulfate, filtered and the solvent removed to afford the title compound as a light yellow solid (975 mg, 97%). No further purification was undertaken.

¹H NMR (400 MHz, CDCl₃): δ ppm 1.4 (d, 6H), 1.6 (s, 9H), 5.30 (m, 1H), 6.60 (m, 1H), 7.40 (s, 1H), 7.65 (m, 1H), 7.80 (s, 1H).

LCMS Rt=2.00 minutes MS m/z 398 [M−H]⁻

### Preparation 44

### 4-((5-chloro-6-isopropoxypyridin-3-yl)oxy)-2,5-difluorobenzoic acid

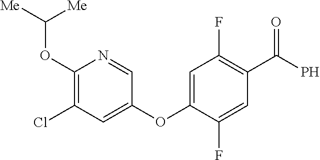

To a solution of tert-butyl 4-((5-chloro-6-isopropoxypyridin-3-yl)oxy)-2,5-difluorobenzoate (Preparation 43, 975 mg, 2.44 mmol) in a mixture tetrahydrofuran/methanol (1:1, 20 mL) was added an aqueous solution of sodium hydroxide (3M, 8.1 mL, 24.4 mmol). The reaction was heated at 65° C. for 3 hours, cooled to room temperature and diluted with ethyl acetate (20 mL). An aqueous solution of hydrochloric acid (1M, 50 mL) was added to reach pH 1. The organic phase was extracted with ethyl acetate (3 ×10 mL) and the combined organic layers were washed with brine (20 mL), dried over magnesium sulfate, filtered and the solvent removed in vacuo to afford the title compound as a white solid (776 mg, 93%). No further purification was undertaken.

¹H NMR (400 MHz, d₆-DMSO): δ ppm 1.35 (d, 6H), 5.20 (m, 1H), 7.05 (m, 1H), 7.80 (m, 1H), 7.95 (s, 1H), 8.05 (s,1H).

LCMS Rt=3.42 minutes MS m/z 342 [M−H]⁻

### Preparation 45

### 4-Methylphenyl 2,4,5-trifluorobenzoate

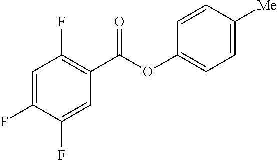 [SMILES: Cc1ccc(OC(=O)c2cc(F)c(F)cc2F)cc1]

Thionyl chloride (50 mL, 680 mmol) was added to 2,4,5-trifluorobenzoic acid (10 g, 57 mmol) and the mixture stirred at 55° C. for 18 hours. After cooling, the excess thionyl chloride was removed in vacuo. The resulting crude oil was azeotroped twice with DCM (30 mL) and toluene (20 mL) and the residue redissolved in DCM (50 mL), then cooled to 0° C. A mixture of 4-methylphenol (6.4 g, 59 mmol) and triethylamine (10 mL, 71 mmol) in DCM (20 mL) was added over 30 minutes. The reaction was allowed to warm up to room temperature over 1 hour. The crude reaction mixture was partitioned between EtOAc (200 mL) and saturated sodium bicarbonate solution (70 mL). The aqueous layer was further extracted with EtOAc (100 mL). The combined organic extracts were combined, washed with saturated sodium bicarbonate solution (70 mL) and water (100 mL). The organic layer was dried over magnesium sulfate and concentrated to provide a crude solid, which was purified by silica gel chromatography eluting with 5% EtOAc in heptane to provide the title compound (10.08 g, 66%) as a white solid.

The title compound can also be prepared according to the following method: 4-methylphenol (80.0 g, 739.8 mmol) was added to a suspension of 2,4,5-trifluorobenzoic acid (136.8 g, 776.8 mmol) and 1,1-carbonyldiimidazole (83-85% wt, 163.6 g, 849.7 mmol) in EtOAc (1.20 L) at 40° C. The reaction mixture was stirred at 40° C. for 2 hours, then cooled to 20° C. and washed with water (480 mL), a 0.5 M aqueous solution of sodium hydroxide (2×400 mL) and water (400 mL). The organics were concentrated in vacuo and azeotroped with heptane to give a yellow oil. Heptane (640 mL) was added and the reaction was stirred at room temperature for 16 hours. A seed was used to facilitate the formation of a suspension. The resulting suspension was cooled to 10° C. and filtered. The residue was washed with cold heptane (80 mL) and dried to afford the title compound as an off white solid (147.5 g, 75%):

¹H NMR (400 MHz, CDCl₃): δ 2.40 (s, 3H), 7.10 (m, 3H), 7.24 (m, 2H), 7.95 (m, 1H).

LCMS Rt=3.53 minutes

### Preparation 46

### Azetidine-1-sulfonamide

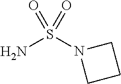

A mixture of palladium hydroxide 20% (350 mg), benzyl (azetidin-1-ylsulfonyl)carbamate (Preparation 47, 1.49 g, 5.5 mmol) and 1-methyl-1,4-cyclohexadiene (10.7 g, 0.11mol) in methanol (35 mL) was stirred and heated at 60° C. overnight under nitrogen. The reaction mixture was cooled to room temperature, passed through a pad of celite and concentrated in vacuo to afford the title compound (437 mg, 58%) as a solid.

¹H NMR (400 MHz, CD₃OD) δ ppm 2.15 (pent, 2H), 3.78 (t, 4H).

MS m/z no mass ion observed

### Preparation 47

### Benzyl (azetidin-1-ylsulfonyl)carbamate

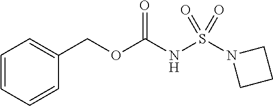 [SMILES: O=C(NS(=O)(=O)N1CCC1)OCc1ccccc1]

Azetidine (0.36 g, 0.5 mmol) was added to N-{1-[N-(benzyloxycarbonyl)-sulfamoyl]pyridin-4(1H)-ylidene}-N-methylmethanaminium chloride (Preparation 48, 2.0 g, 0.5 mmol) in DCM (10 mL). The reaction mixture was stirred overnight at room temperature. The mixture was concentrated in vacuo and the residue partitioned between ethyl acetate (50 mL) and water (50 mL). The organic layer was discarded and the aqueous layer was acidified with 1M HCl. The aqueous layer was extracted with ethylacetate (2×50 mL), dried over magnesium sulfate and concentrated to afford the title compound (1.49 g, 100%).

¹H NMR (400 MHz, CDCl₃) δ ppm 2.20 (pent, 2H), 4.10 (t, 4H), 5.22 (s, 2H), 7.39 (m, 5H).

LCMS Rt=1.93 minutes MS m/z 271 [MH]⁺

### Preparation 48

### N-{1-[N-(benzyloxycarbonyl)sulfamoyl]pyridin-4(1H)-ylidene}-N-methylmethanaminium chloride

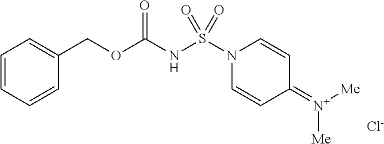 [SMILES: C[N+](C)=c1ccn(S(=O)(=O)NC(=O)OCc2ccccc2)cc1 ; [Cl-]]

Chlorosulfonylisocyanate (5.85 mL, 67.0 mmol) was added slowly over 20 min, to a stirred solution of benzylalcohol (7.05 g, 67.0 mmol) in DCM (80 mL) at 0° C. After 30 min DMAP (16.5g, 0.13 mol) was added portion-wise, keeping the temperature between 0 and 5° C. The reaction was allowed to warm to room temperature over 3 hours. Water (40 mL) was added carefully to the mixture and the layer separated. The organic layer was washed with water (40 mL), dried over magnesium sulfate, filtered and concentrated in vacuo to yield a solid. The solid was recrystalized from acetonitrile (150 mL) to provide the title compound (11.9 g, 55%) as a white solid.

¹H NMR (400 MHz, CD₃OD) δ ppm 3.25 (s, 6H), 4.95 (s, 2H), 6.84 (d, 2H), 7.31 (m, 5H), 8.48 (d, 2H).

LCMS Rt=1.60 minutes MS m/z 337 [MH]⁺

### Preparation 49

### tert-butyl 5-chloro-4-(3,4-dichlorophenoxy)-2-fluorobenzoate

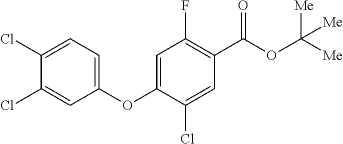 [SMILES: CC(C)(C)OC(=O)c1cc(Cl)c(Oc2ccc(Cl)c(Cl)c2)cc1F]

tert-Butyl 5-chloro-2,4-difluorobenzoate (WO2012007861, 1.60 g, 6.44 mmol) and potassium carbonate (1.69 g, 12.26 mmol) were added to a solution of 3,4-dichlorophenol (1 g, 6.13 mmol) in DMSO (30 mL). The mixture was stirred at room temperature for 16 hours. The mixture was diluted with EtOAc (100 mL) then washed with water (100 mL). The organic layer was dried over MgSO₄ and the filtrate was evaporated to give the title compound as an orange gum (2.40 g, 100% yield).

¹H NMR (400 MHz, CDCl₃): δ 1.58 (s, 9H), 6.65 (d, 1H), 6.90 (dd, 1H), 7.14 (d, 1H), 7.46 (d, 1H), 7.98 (d, 1H) ppm.

¹⁹F NMR (376 MHz, CDCl₃): δ −107.11 (s) ppm.

LCMS Rt=4.45 minutes

### Preparation 50

### 5-chloro-4-(3,4-dichlorophenoxy)-2-fluorobenzoic acid

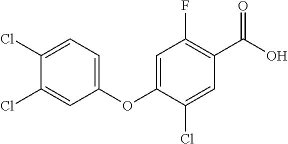 [SMILES: O=C(O)c1cc(Cl)c(Oc2ccc(Cl)c(Cl)c2)cc1F]

Trifluoroacetic acid (4.00 mL, 52.09 mmol) was added to a solution of tert-butyl 5-chloro-4-(3,4-dichlorophenoxy)-2-fluorobenzoate (Preparation 49, 2.40 g, 6.13 mmol) in CH₂Cl₂ (40 mL). The reaction mixture was heated at 40° C. for 3 hours. The solvent was evaporated under reduced pressure and the residue was co-evaporated with CH₂Cl₂. Then crude was triturated with heptane to give title compound as a solid (1.79 g, 87% yield).

¹H NMR (400 MHz, CDCl₃): δ 6.65 (d, 1H), 6.96 (dd, 1H), 7.21 (d, 1H), 7.51 (d, 1H), 8.16 (d, 1H) ppm.

¹⁹F NMR (376 MHz, CDCl₃): δ −104.76 (s, 1F) ppm

LCMS Rt=2.60 minutes MS m/z 335 [MH]⁺

The ability of the compounds of formula (I) to block the Nav1.7 (or SCN9A) channel were measured using the assay described below.

Cell Line Construction and Maintenance

Human Embryonic Kidney (HEK) cells were transfected with an hSCN9A construct using lipofectamine reagent (Invitrogen), using standard techniques. Cells stably expressing the hSCN9A constructs were identified by their resistance to G-418 (400 μg/ml). Clones were screened for expression using the whole-cell voltage-clamp technique.

Cell Culture

HEK cells stably transfected with hSCN9A were maintained in DMEM medium supplemented with 10% heat-inactivated fetal bovine serum and 400 μg/ml G-418 in an incubator at 37° C. with a humidified atmosphere of 10% CO₂. For HTS, cells were harvested from flasks by trypsinization and replated in an appropriate multi-well plate (typically 96 or 384 wells/plate) such that confluence would be achieved within 24 hours of plating. For electrophysiological studies, cells were removed from the culture flask by brief trypsinization and re-plated at low density onto glass cover slips. Cells were typically used for electrophysiological experiments within 24 to 72 hours after plating.

Electrophysiological Recording

Cover slips containing HEK cells expressing hSCN9A were placed in a bath on the stage of an inverted microscope and perfused (approximately 1 ml/minutes) with extracellular solution of the following composition: 138 mM NaCl, 2 mM CaCl₂, 5.4 mM KCl, 1mM MgCl₂, 10 mM glucose, and 10 mM HEPES, pH 7.4, with NaOH. Pipettes were filled with an intracellular solution of the following composition: 135 mM CsF, 5 mM CsCl, 2 mM MgCl₂, 10 mM EGTA, 10 mM HEPES, pH 7.3 with NaOH, and had a resistance of 1 to 2 megaohms. The osmolarity of the extracellular and intracellular solutions was 300 mOsm/kg and 295 mOsm/kg, respectively. All recordings were made at room temperature (22-24° C.) using AXOPATCH 200B amplifiers and PCLAMP software (Axon Instruments, Burlingame, Calif.).

hSCN9A currents in HEK cells were measured using the whole-cell configuration of the patch-clamp technique (Hamill et al., 1981). Uncompensated series resistance was typically 2 to 5 mega ohms and >85% series resistance compensation was routinely achieved. As a result, voltage errors were negligible and no correction was applied. Current records were acquired at 20 to 50 KHz and filtered at 5 to 10 KHz.

HEK cells stably transfected with hSCN9A were viewed under Hoffman contrast optics and placed in front of an array of flow pipes emitting either control or compound-containing extracellular solutions. All compounds were dissolved in dimethyl sulfoxide to make 10 mM stock solutions, which were then diluted into extracellular solution to attain the final concentrations desired. The final concentration of dimethyl sulfoxide (<0.3% dimethyl sulfoxide) was found to have no significant effect on hSCN9A sodium currents. The voltage-dependence of inactivation was determined by applying a series of depolarizing prepulses (8 sec long in 10 mV increments) from a negative holding potential. The voltage was then immediately stepped to 0 mV to assess the magnitude of the sodium current. Currents elicited at 0 mV were plotted as a function of prepulse potential to allow estimation of the voltage at which 50% of the channels were inactivated (midpoint of inactivation or V1/2). Compounds were tested for their ability to inhibit hSCN9A sodium channels by activating the channel with a 20 msec voltage step to 0 mV following an 8 second conditioning prepulse to the empirically determined V1/2. Compound effect (% inhibition) was determined by difference in current amplitude before and after application of test compounds. For ease of comparison, “estimated IC-50” (EIC₅₀) values were calculated from single point electrophysiology data by the following equation, (tested concentration, uM)×(100-% inhibition/% inhibition). Inhibition values <20% and >80% were excluded from the calculation.

Electrophysiological assays were conducted with PatchXpress 7000 hardware and associated software (Molecular Devices Corp). All assay buffers and solutions were identical to those used in conventional whole-cell voltage clamp experiments described above. hSCN9A cells were grown as above to 50%-80% confluency and harvested by trypsinization. Trypsinized cells were washed and resuspended in extracellular buffer at a concentration of 1×10⁶ cells/ml. The onboard liquid handling facility of the PatchXpress was used for dispensing cells and application of test compounds. Determination of the voltage midpoint of inactivation was as described for conventional whole-cell recordings. Cells were then voltage-clamped to the empirically determined V1/2 and current was activated by a 20 msec voltage step to 0 mV.

Electrophysiological assays may also be conducted using the Ionworks Quattro automated electrophysiological platform (Molecular Devices Corp). Intracellular and extracellular solutions were as described above with the following changes, 100 μg/ml amphotericin was added to the intracellular solution to perforate the membrane and allow electrical access to the cells. hSCN9A cells were grown and harvested as for PatchXpress and cells were resuspended in extracellular solution at a concentration of 3-4×10⁶ cells/ml. The onboard liquid handling facility of the Ionworks Quattro was used for dispensing cells and application of test compounds. A voltage protocol was then applied that comprised of a voltage step to fully inactivate the sodium channels, followed by a brief hyperpolarized recovery period to allow partial recovery from inactivation for unblocked sodium channels, followed by a test depolarized voltage step to assess magnitude of inhibition by test compound. Compound effect was determined based on current amplitude difference between the pre-compound addition and post-compound addition scans.

Compounds of the Examples were tested in the assay described above using the PatchXpress platform and found to have the Nav1.7 EIC₅₀ (uM) values specified in the table below.

| Ex. | EIC₅₀ |
|---|---|
| 1 | 2.6 |
| 2 | 0.13 |
| 3 | 4.8 |
| 4 | >3 |
| 5 | 7.1 |
| 6 | 7.3 |
| 7 | >3 |
| 8 | >30 |
| 9 | >30 |
| 10 | 6.3 |
| 11 | 2.1 |
| 12 | >1 |
| 13 | >1 |
| 14 | 7.5 |
| 15 | >3 |
| 16 | 4.9 |
| 17 | 0.20 |
| 18 | 34 |
| 19 | 10 |
| 20 | 1.2 |
| 21 | 0.60 |
| 22 | 2.5 |
| 23 | 0.67 |
| 24 | 0.66 |
| 25 | 9.8 |
| 26 | 0.48 |
| 27 | 3.0 |
| 28 | 0.16 |
| 29 | 0.24 |
| 30 | 0.47 |
| 31 | 6.6 |
| 32 | 0.29 |
| 33 | 0.50 |
| 34 | 0.59 |
| 35 | 2.5 |
| 36 | 0.66 |
| 37 | 0.47 |
| 38 | 0.18 |
| 39 | 30 |
| 40 | NT |
| 41 | 0.10 |
| 42 | 0.10 |

The ability of compounds of formula (I) to block the Nav1.5 (or SCN5A) channel can also be measured using an assay analogous to that described above but replacing the SCN9A gene with the SCN5A gene. All other conditions remain the same including the same cell line and conditions for cell growth. The estimated IC50s are determined at the half inactivation for Nav1.5. These results can be compared to the EIC₅₀ value at the Nav1.7 channel to determine the selectivity of a given compound for Nav1.7 vs Nav1.5.

1. A compound of formula (I): 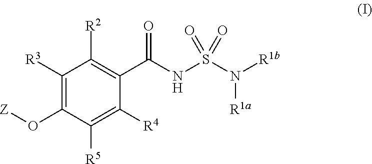
or a pharmaceutically acceptable salt thereof, wherein
Z is selected from naphthyl, phenyl or Het¹, wherein said naphthyl, phenyl or Het¹ is optionally substituted by one to three substituents selected from Y¹ or Y²; wherein Y¹ and Y² are each independently selected from:
(i) F;
(ii) Cl;
(iii) CN;
(iv) (C₁-C₈)alkyl, optionally substituted by (C₃-C₈)cycloalkyl and/or by one to eight F;
(v) (C₃-C₈)cycloalkyl, optionally substituted by one to eight F;
(vi) NR⁷R⁸;
(vii) (C₁-C₈)alkyloxy, optionally substituted by one to three R⁹, and/or by one to eight F;
(viii) (C₃-C₈)cycloalkyloxy, optionally substituted by one to eight F and/or by one to three R¹⁰, and further optionally fused to a phenyl ring;
(ix) (phenyl, optionally substituted by one to three substituents selected from F or R¹⁰;
(x) (phenoxy, optionally substituted by one to three substituents selected from F or R¹⁰;
(xi) Het²;
(xii) Het²-oxy; or
(xiii) Het³;
R^{1a}and R^{1b} are each independently selected from:
(i) H,
(ii) (C₁-C₆)alkyl; or
(iii) (C₃-C₆)cycloalkyl, optionally substituted by one to eight F or, R^{1a} and R^{1b} taken together with the N atom to which they are attached, form a 3- to 8-membered monoheterocycloalkyl, said monoheterocycloalkyl being optionally substituted on a ring carbon atom by valency permitting, one to eight F;
R², R³, and R⁴ are each independently selected from H, F, Cl or —OCH₃;
R⁵ is H, CN, F, Cl, Het³, or R⁶; wherein R⁶ is selected from (C₁-C₆)alkyl or (C₁-C₆)alkyloxy, and said (C₁-C₆)alkyl and C₁-C₆)alkyloxy are optionally substituted by one to eight F;
R⁷ and R⁸ are each independently selected from:
(i) H,
(ii ) (C₁-C₈)alkyl, optionally substituted by one to three R¹¹;
(iii) (C₃-C₈)cycloalkyl, optionally substituted by one to eight F and/or by one to three R¹⁰, and further optionally fused to a phenyl ring;
(iv) ‘C-linked’ Het²; or
(v) C-linked Het³;
R⁹ is selected from:
(i) (C₁-C₆)alkyloxy;
(ii) (C₃-C₈)cycloalkyl, optionally substituted by one to eight F;
(iii) Het²; or
(iv) phenyl, optionally substituted by one to three R⁶;
R¹⁰ is Cl, CN or R⁶;
R¹¹ is selected from:
(i) F;
(ii) (C₁-C₆)alkyloxy;
(iii) (C₃-C₈)cycloalkyl, optionally substituted by one to eight F;
(iv) ‘C-linked’ Het²; or
(v) phenyl, optionally substituted by one to three R⁶;
Het¹ is a 6-, 9- or 10-membered heteroaryl containing one to three nitrogen atoms;
Het² is a 3- to 8-membered saturated monoheterocycloalkyl containing one or two ring members selected from —NR¹²—and —O—, said monoheterocycloalkyl being optionally substituted on a ring carbon atom by one to three substituents independently selected from F, (C₁-C₆)alkyl, (C₁-C₄)alkyloxy(C₀-C₄)alkylene or (C₃-C₈)cycloalkyl;
Het³ is a 5- or 6-membered heteroaryl containing one to three nitrogen atoms, said heteroaryl being optionally substituted by one to three substituents selected from F, Cl, CN or R⁶; and
R¹² is H, (C₁-C₆)alkyl or (C₃-C₈)cycloalkyl, wherein said (C₁-C₆)alkyl and said (C₃-C₈)cycloalkyl are optionally substituted by one to eight F; or, when Het² is ‘N-linked’, R¹² is absent.

2. The compound according to claim 1, or a pharmaceutically acceptable salt thereof wherein Z is phenyl optionally substituted by one to three substituents selected from Y¹ and Y².

3. The compound according to claim 1 wherein Z is phenyl optionally substituted by one to two substituents selected from Y¹ or Y².

4. The compound according to claim 3 wherein said phenyl is meta and para substituted.

5. The compound according to claim, or a pharmaceutically acceptable salt thereof 1 wherein Z is a 6-membered heteroaryl comprising one to three nitrogen atoms, said heteroaryl being optionally independently substituted by one to three substituents selected from Y¹ or Y².

6. The compound according to claim 1, or a pharmaceutically acceptable salt thereof wherein Z is pyridyl optionally substituted by one to three substituents selected from Y¹ and or Y².

7. The compound according to claim 1, or a pharmaceutically acceptable salt thereof wherein Z is pyridyl optionally independently substituted by one or two substituents selected from Y¹ and Y² and wherein said pyridyl is orientated as below:

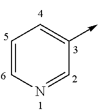

8. The compound according to claim 7 wherein said pyridyl is 6-substituted or 5- and 6-disubstituted.

9. The compound according to claim 1, or a pharmaceutically acceptable salt thereof wherein Y¹ and Y² are each independently selected from:
(i) F;
(ii) Cl;
(iii) (C₁-C₆)alkyl, optionally substituted by one to six F;
(iv) NR⁷R⁸ wherein R⁷ and R⁸ are each independently selected from H, (C₁-C₆)alkyl, (C₃-C₆)cycloalkyl, (C₅-C₈)bridged bicycloalkyl or (C₁-C₆)alkyl substituted by (C₃-C₆)cycloalkyl; or R⁷ and R⁸ taken together with the N atom to which they are attached form a 3- to 8-membered monoheterocycloalkyl, said monoheterocycloalkyl being optionally substituted on a ring carbon atom by (C₁-C₆)alkyl and/or one to two F;
(v) (C₁-C₈)alkyloxy, optionally substituted by (C_{3—}C₆)cycloalkyl, and/or one to eight F;
(vi) phenyl; or
(vii) Het³.

10. The compound according to claim 1, or a pharmaceutically acceptable salt thereof wherein Y¹ and Y² are each independently selected from:
(i) Cl;
(ii) (C₁-C₂)alkyl, optionally substituted by one to three F;
(iii) NR⁷R⁸ wherein R⁷ and R⁸ are each independently selected from H, (C₁-C₃)alkyl, (C₃-C₄)cycloalkyl or (C₁-C₃)alkyl substituted by (C₃-C₄)cycloalkyl; or
(iv) (C₁-C₄)alkyloxy, optionally substituted by one to six F.

11. The compound to claim 1, or a pharmaceutically acceptable salt thereof wherein R^{1a} and R^{1b} are each independently selected from H or (C₁-C₃)alkyl; or, R^{1a} and R^{1b} taken together with the N atom to which they are attached, form a 3- to 6-membered monoheterocycloalkyl, said monoheterocycloalkyl being optionally substituted on a ring carbon atom by one or two F.

12. The compound according to claim 1, or a pharmaceutically acceptable salt thereof wherein R^{1a} and R^{1b} are independently selected from H or methyl; or, R^{1a} and R^{1b} taken together with the N atom to which they are attached, form a 3- to 6-membered monoheterocycloalkyl.

13. The compound according to claim 1, or a pharmaceutically acceptable salt thereof wherein R², R³, R⁴ and R⁵ are each independently H, F or Cl.

14. The compound according to claim 1, or a pharmaceutically acceptable salt thereof wherein R² is F, R³ and R⁴ are both H; and R⁵ is F or Cl.

15. A pharmaceutical composition comprising a compound according to claim 1 or a pharmaceutically acceptable salt thereof together with one or more pharmaceutically acceptable excipients.

16. The pharmaceutical composition according to claim 15, wherein said composition further comprises one or more additional therapeutic agents.

17-20. (canceled)

21. A method of treating a disorder in a human or animal for which a Nav1.7 inhibitor is indicated, comprising administering to said human or animal a therapeutically effective amount of a compound according to claim 1 or a pharmaceutically acceptable salt thereof.

22. The method according to claim 21, wherein the disorder for which a Nav1.7 inhibitor is indicated is pain.

23. The method according to claim 22, wherein said pain is selected from neuropathic, nociceptive or inflammatory pain.
